(19) United States
(12) Patent Application Publication
(10) Pub. No.: US 2026/0015546 A1
(43) Pub. Date: Jan. 15, 2026

(54) ASSEMBLIES, SYSTEMS, APPARATUSES, AND PROCESSES FOR ENHANCING FRACTIONATION AND DISTILLATION PROCESSES ASSOCIATED WITH REFINING OPERATIONS

(71) Applicant: Marathon Petroleum Company LP, Findlay, OH (US)

(72) Inventors: Roy Roger Bledsoe, JR., Findlay, OH (US); James F. Johnson, Findlay, OH (US); Ross T. Vail, Findlay, OH (US); Jeffrey A. Sexton, Findlay, OH (US); Chuck R. Johnston, Findlay, OH (US); Joshua D. Santrock, Findlay, OH (US); Steven M. Williams, Findlay, OH (US)

(21) Appl. No.: 19/333,973

(22) Filed: Sep. 19, 2025

Related U.S. Application Data
(63) Continuation-in-part of application No. 17/652,431, filed on Feb. 24, 2022, now Pat. No. 12,461,022.
(60) Provisional application No. 63/153,452, filed on Feb. 25, 2021.
(60) Provisional application No. 63/268,456, filed on Feb. 24, 2022.
(60) Provisional application No. 63/697,028, filed on Sep. 20, 2024.

(51) Int. Cl. C10G 7/12; C10G 7/06; G01N 21/3577; G01N 21/359; G01N 21/65; G01N 33/28

(52) U.S. Cl. CPC C10G 7/12; C10G 7/06; G01N 21/3577; G01N 21/359; G01N 21/65; G01N 33/28; C10G 2300/1074; C10G 2300/4006

(57) ABSTRACT

Assemblies, systems, apparatuses, and processes to enhance a fractionation or distillation process associated with a refining operation may include analyzing, via one or more spectroscopic analyzers, samples of a hydrocarbon feedstock supplied to one or more processing units associated with the refining operation, unit materials, and/or downstream materials. The assemblies, systems, and processes further may include prescriptively controlling, via one or more process controllers, based at least in part on feedstock properties, unit material properties, and/or downstream material properties, the one or more processing units, so that the prescriptively controlling results in causing the fractionation or distillation process to produce unit materials and/or downstream materials having properties within selected ranges of target properties, thereby to cause the fractionation or distillation process to achieve material outputs that more accurately and responsively converge on one or more of the target properties.

### PRIORITY CLAIMS

This application claims priority to and the benefit of U.S. Provisional Application No. 63/697,028, filed Sep. 20, 2024, titled “ASSEMBLIES, SYSTEMS, APPARATUSES, AND PROCESSES FOR ENHANCING FRACTIONATION AND DISTILLATION PROCESSES ASSOCIATED WITH REFINING OPERATIONS” and is a continuation-in-part of U.S. Non-Provisional application Ser. No. 17/652,431, filed Feb. 24, 2022, titled “METHODS AND ASSEMBLIES FOR DETERMINING AND USING STANDARDIZED SPECTRAL RESPONSES FOR CALIBRATION OF SPECTROSCOPIC ANALYZERS,” which claims priority to and the benefit of U.S. Provisional Application No. 63/153,452, filed Feb. 25, 2021, titled “METHODS AND ASSEMBLIES FOR DETERMINING AND USING STANDARDIZED SPECTRAL RESPONSES FOR CALIBRATION OF SPECTROSCOPIC ANALYZERS,” and U.S. Provisional Application No. 63/268,456, filed Feb. 24, 2022, titled “ASSEMBLIES AND METHODS FOR ENHANCING CONTROL OF FLUID CATALYTIC CRACKING (FCC) PROCESSES USING SPECTROSCOPIC ANALYZERS,” the disclosures of all of which are incorporated herein by reference in their entireties.

### TECHNICAL FIELD

The present disclosure relates to assemblies, systems, apparatuses, and processes for enhancing distillation processes and, more particularly, to assemblies, systems, apparatuses, and processes for enhancing fractionation and distillation processes associated with refining operations.

### BACKGROUND

Refining operations may include refinery processes that may be used to produce desired petroleum-based intermediate and final products from hydrocarbon feeds. Many refinery processes are inherently complex, often involving a large number of variables and processing parameters associated with the hydrocarbon feeds and operation of multiple processing units for transforming the hydrocarbon feed into desired intermediate and final products. Design, control, and optimization of the processing units may benefit from analytical models that describe conversion of hydrocarbon feeds to products. Analytical models, however, may only be useful if provided with timely and accurate information. If the information lacks sufficient accuracy, the analytical model may provide inaccurate outputs, for example, relating to hydrocarbon feedstock monitoring and control, and/or control of the processing units, and resulting products may lack desired properties. If the information is not provided to the analytical model in a sufficiently responsive manner, desired changes based on the information and model outputs may be delayed, resulting in extending the time during which the processes are performed below optimum efficiency. Conventional laboratory analysis of the hydrocarbon feeds and related materials or processes may suffer from insufficiently responsive results to provide effective monitoring and control of the processes and related materials. For example, off-line laboratory analysis and related modeling studies may involve response times of hours, days, or even weeks, during which processing parameters are not optimized. As a result, the value of such monitoring and control may be reduced when used to monitor and control the processes in during operation.

Although some refinery processes may include devices and strategies for monitoring and controlling the refinery process, Applicant has recognized that such devices and strategies may suffer from delayed acquisition of useful information and/or inaccuracies due to the nature of the devices or strategies, for example, when an analysis is manually commenced and/or laboratory evaluation is performed. As a result, Applicant has recognized that there may be a desire to provide assemblies, systems, apparatuses, and methods for more accurately monitoring, controlling, and/or optimizing refinery processes and/or for more responsively determining properties and/or characteristics of hydrocarbon feeds, processing unit product materials, intermediate materials, upstream materials, and/or downstream materials related to the refinery processes. Such assemblies, systems, apparatuses, and methods may result in enhanced control or optimization of refinery processes for more efficiently producing refinery products.

The present disclosure may address one or more of the above-referenced considerations, as well as other possible considerations.

### SUMMARY

The present disclosure is generally directed to embodiments of assemblies, systems, apparatuses, and methods for enhancing distillation processes and, more particularly, to assemblies, systems, apparatuses, and processes for enhancing distillation processes associated with refining operations. Monitoring and control of distillation processes may be important for producing intermediate and final products having certain characteristics or properties to meet industry and/or marketing standards. Using current systems and processes, it may be difficult to achieve desired standards because the systems and methods may suffer from delayed acquisition of useful information and/or inaccuracies due to the nature of the devices or processes. At least some embodiments of the present disclosure may advantageously provide assemblies, systems, apparatuses, and/or methods for monitoring, controlling, and/or optimizing distillation processes, such that the resulting intermediate and final products have desired characteristics or properties that may be achieved more efficiently. In some embodiments, the assemblies, systems, apparatuses, and/or methods disclosed herein may result in acquisition of useful information and/or provide more accurate information for monitoring, controlling, and/or optimizing distillation processes, in some instances, while the distillation processes are occurring. This, in turn, may result in producing intermediate and final products having desired characteristics or properties in a more efficient manner. For example, in at least some embodiments, at least some of the acquired information may be used to monitor and prescriptively control distillation processes, during the distillation processes, resulting in producing intermediate and final products having desired characteristics or properties in a more economically efficient manner. For example, prescriptively controlling the distillation process assembly and/or the distillation process, during the distillation processes, according to some embodiments, may result in causing the distillation process to produce intermediate materials, unit materials, and/or downstream materials having properties within selected ranges of target properties, thereby to cause the distillation process to achieve material outputs that more accurately and responsively converge on one or more of the target properties.

In some embodiments, a method for enhancing control of a refining process associated with a petroleum refining operation may include conditioning a hydrocarbon feedstock sample of a hydrocarbon feedstock supplied to one or more first processing units associated with the petroleum refining operation, the conditioning including one or more of: (a) filtering the hydrocarbon feedstock sample, (b) changing a temperature of the hydrocarbon feedstock sample, (c) diluting the hydrocarbon feedstock sample in solvent, or (d) degassing the hydrocarbon feedstock sample. The hydrocarbon feedstock may have one or more hydrocarbon feedstock properties, and the one or more first processing units may include one or more fractionation units. In some embodiments, the method may include operating the one or more first processing units to produce one or more unit materials having one or more unit materials properties. The unit materials may include one or more of intermediate materials or unit product materials. The method further may include analyzing the hydrocarbon feedstock sample via a first spectroscopic analyzer to provide hydrocarbon feedstock sample spectra. The method also may include predicting one or more hydrocarbon feedstock sample properties based at least in part on the hydrocarbon feedstock sample spectra.

In some embodiments, the method also may include conditioning a unit material sample from the one or more unit materials to one or more of filter the unit material sample, change a temperature of the unit material sample, dilute the unit material sample in solvent, or degas the unit material sample. The method may further include analyzing the unit material sample via one or more of the first spectroscopic analyzer or a second spectroscopic analyzer to provide unit material sample spectra, and predicting one or more unit material sample properties based at least in part on the unit material sample spectra. The one or more of the first spectroscopic analyzer or the second spectroscopic analyzer may be calibrated to generate standardized spectral responses.

The method may further include prescriptively controlling, during the refining process, via one or more controllers based at least in part on the one or more hydrocarbon feedstock sample properties and the one or more unit material sample properties, one or more of: (a) the one or more hydrocarbon feedstock parameters associated with the hydrocarbon feedstock supplied to the one or more first processing units; (b) one or more intermediates properties associated with the intermediate materials produced by the one or more first processing units; (c) one or more unit product materials properties associated with the unit product materials; (d) operation of the one or more first processing units; or (e) operation of one or more second processing units positioned downstream relative to the one or more first processing units. The prescriptively controlling may cause the refining process to produce one or more of: (a) one or more intermediate materials each having one or more properties within a range of one or more target properties of the one or more intermediate materials; (b) one or more unit product materials each having one or more properties within a range of one or more target properties of the one or more unit product materials; or (c) one or more downstream materials each having one or more properties within a range of one or more target properties of the one or more downstream materials. The prescriptively controlling thereby may cause the refining process to achieve material outputs that more accurately and prescriptively converge on one or more of the target properties.

In some embodiments, a distillation unit control assembly to enhance control of a refining process associated with a petroleum refining operation may include a first spectroscopic analyzer positioned to receive a hydrocarbon feedstock sample of a hydrocarbon feedstock supplied to one or more first processing units associated with the petroleum refining operation. The one or more first processing units may include one or more fractionation units. The first spectroscopic analyzer may also be positioned to analyze the hydrocarbon feedstock sample to provide hydrocarbon feedstock sample spectra, and predict one or more hydrocarbon feedstock sample properties associated with the hydrocarbon feedstock sample based at least in part on the hydrocarbon feedstock sample spectra. The distillation unit control assembly also may include a second spectroscopic analyzer positioned to receive a unit material sample of one or more unit materials produced by the one or more first processing units, analyze the unit material sample to provide unit material sample spectra, and predict one or more unit material sample properties associated with the unit material sample based at least in part on the unit material sample spectra.

The distillation unit control assembly further may include a sample conditioning assembly positioned to one or more of condition the hydrocarbon feedstock sample prior to being supplied to the first spectroscopic analyzer or condition the unit material sample prior to being supplied to the second spectroscopic analyzer. The sample conditioning assembly may be configured to one or more of: filter, change a temperature, dilute in solvent, or degas one or more of the hydrocarbon feedstock sample or the unit material sample.

In some embodiments, the distillation unit control assembly also may include a process controller in communication with the first spectroscopic analyzer and the second spectroscopic analyzer. The process controller may be configured to, during the refining process, prescriptively control, based at least in part on the one or more hydrocarbon feedstock sample properties and the one or more unit material sample properties, one or more of: (a) one or more hydrocarbon feedstock parameters associated with the hydrocarbon feedstock supplied to the one or more first processing units; (b) the one or more hydrocarbon feedstock properties associated with the hydrocarbon feedstock supplied to the one or more first processing units; (c) one or more intermediates properties associated with the intermediate materials produced by the one or more first processing units; (d) one or more unit materials properties associated with the one or more unit materials; (e) operation of the one or more first processing units; or (f) operation of one or more second processing units positioned downstream relative to the one or more first processing units.

The prescriptively controlling during the refining process may cause the refining process to produce one or more of: (a) one or more intermediate materials each having one or more properties within a selected range of one or more target properties of the one or more intermediate materials, (b) one or more unit materials each having one or more properties within a selected range of one or more target properties of the one or more unit materials, or (c) one or more downstream materials each having one or more properties within a selected range of one or more target properties of the one or more downstream materials. The refining process may thereby achieve material outputs that more accurately and responsively converge on one or more of the target properties.

In some embodiments, a distillation unit control assembly to enhance control of a refining process associated with a petroleum refining operation may be in communication with one or more spectroscopic analyzers and one or more first processing units including one or more fractionation units. The distillation unit control assembly may be configured to predict one or more hydrocarbon feedstock sample properties associated with a hydrocarbon feedstock sample based at least in part on hydrocarbon feedstock sample spectra generated by the one or more spectroscopic analyzers. The distillation unit control assembly also may be configured to predict one or more unit material sample properties associated with a unit material sample based at least in part on unit material sample spectra generated by the one or more spectroscopic analyzers. The one or more unit material sample properties may be associated with one or more unit materials produced by the one or more first processing units and may include one or more of intermediate materials or unit product materials.

In some embodiments, during the refining process, the distillation unit control assembly may be configured to prescriptively control, based at least in part on the one or more hydrocarbon feedstock sample properties and the one or more unit material sample properties, one or more of: (a) the one or more hydrocarbon feedstock properties associated with hydrocarbon feedstock supplied to the one or more first processing units; (b) one or more intermediates properties associated with the intermediate materials produced by one or more of the first processing units; (c) operation of the one or more first processing units; (d) one or more unit materials properties associated with the one or more unit materials produced by the one or more first processing units; or (e) operation of one or more second processing units positioned downstream relative to the one or more first processing units.

In some embodiments, the prescriptively controlling may cause the refining process to produce one or more of: (a) one or more intermediate materials each having one or more properties within a selected range of one or more target properties of the one or more intermediate materials, (b) one or more unit materials each having one or more properties within a selected range of one or more target properties of the one or more unit materials, or (c) one or more downstream materials each having one or more properties within a selected range of one or more target properties of the one or more downstream materials, thereby to cause the refining process to achieve material outputs that more accurately and responsively converge on one or more of the target properties.

In some embodiments, a distillation unit control assembly for performing a refining process associated with a petroleum refining operation may include one or more first processing units associated with the petroleum refining operation. The one or more first processing units may include one or more fractionation units. The distillation unit control assembly also may include a first spectroscopic analyzer positioned to receive, during the refining process, a hydrocarbon feedstock sample of a hydrocarbon feedstock supplied to the one or more first processing units. The hydrocarbon feedstock may have one or more hydrocarbon feedstock properties. The first spectroscopic analyzer further may be positioned to analyze, during the refining process, the hydrocarbon feedstock sample to provide hydrocarbon feedstock sample spectra, and predict, during the refining process, one or more hydrocarbon feedstock sample properties associated with the hydrocarbon feedstock sample based at least in part on the hydrocarbon feedstock sample spectra.

The distillation unit control assembly also may include a second spectroscopic analyzer positioned to receive, during the refining process, a unit material sample of one more unit materials produced by the one or more first processing units. The first spectroscopic analyzer and the second spectroscopic analyzer may be calibrated to generate standardized spectral responses. The one or more unit materials may include one or more of intermediate materials or unit product materials. The second spectroscopic analyzer further may be positioned to analyze, during the refining process, the unit material sample to provide unit material sample spectra, and predict, during the refining process, one or more unit material sample properties associated with the unit material sample based at least in part on the unit material sample spectra.

In some embodiments, the distillation unit control assembly also may include a sample conditioning assembly positioned to one or more of condition the hydrocarbon feedstock sample prior to being supplied to the first spectroscopic analyzer or condition the unit material sample prior to being supplied to the second spectroscopic analyzer. The sample conditioning assembly may one or more of filter, change a temperature, dilute in solvent, or degas one or more of the hydrocarbon feedstock sample or the unit material sample.

The distillation unit control assembly further may include a distillation unit process controller in communication with the first spectroscopic analyzer and the second spectroscopic analyzer during the refining process. The distillation unit control process controller may be configured to prescriptively control, during the refining process, based at least in part on the one or more hydrocarbon feedstock properties and the one or more unit material sample properties, one or more of: (a) one or more hydrocarbon feedstock parameters associated with the hydrocarbon feedstock supplied to the one or more first processing units; (b) one or more intermediates properties associated with the intermediate materials produced by one or more of the first processing units; (c) operation of the one or more first processing units; (d) one or more unit materials properties associated with the one or more unit product materials; or (e) operation of one or more second processing units positioned downstream relative to the one or more first processing units.

In some embodiments, the prescriptively controlling during the refining process may cause the refining process to produce one or more of: (a) one or more intermediate materials each having one or more properties within a selected range of one or more target properties of the one or more intermediate materials, (b) one or more unit product materials each having one or more properties within a selected range of one or more target properties of the one or more unit product materials, or (c) one or more downstream materials each having one or more properties within a selected range of one or more target properties of the one or more downstream materials, thereby to cause the refining process to achieve material outputs that more accurately and responsively converge on one or more of the target properties.

Still other aspects, examples, and advantages of these exemplary aspects and embodiments are discussed in more detail below. It is to be understood that both the foregoing information and the following detailed description are merely illustrative examples of various aspects and embodiments and are intended to provide an overview or framework for understanding the nature and character of the claimed aspects and embodiments. Accordingly, these and other objects, along with advantages and features of the present disclosure herein disclosed, may become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the exemplary embodiments discussed herein and the various ways in which they may be practiced. According to common practice, the various features of the drawings discussed below are not necessarily drawn to scale. Dimensions of various features and elements in the drawings may be expanded or reduced to more clearly illustrate one or more of the embodiments of the disclosure.

FIG. 1 is a schematic block diagram of an example refining operation with fractionation units including an example atmospheric distillation unit, an example vacuum distillation unit, and an example saturated gas unit, also including example measurement devices to facilitate control of the refining operation, according to embodiments of the disclosure.

FIG. 2 is a schematic diagram of an example crude unit distillation unit and associated hydrocarbon feeds, according to embodiments of the disclosure.

FIG. 3 is a schematic block diagram of an example refining operation with first processing units including fractionation units, and an example distillation process control assembly, according to embodiments of the disclosure.

FIG. 4 is a schematic block diagram of an example sample conditioning assembly, according to embodiments of the disclosure.

FIG. 5A is a block diagram illustrating an example process for enhancing refinery processes using a controller and spectroscopic analyzer, according to embodiments of the disclosure.

FIG. 5B is a continuation of the block diagram shown in FIG. 5A, according to embodiments of the disclosure.

FIG. 5C is a continuation of the block diagram shown in FIG. 5A and FIG. 5B, according to embodiments of the disclosure.

FIG. 5D is a continuation of the block diagram shown in FIG. 5A, FIG. 5B, and FIG. 5C, according to embodiments of the disclosure.

FIG. 6 is a schematic diagram of an example process controller configured to at least partially control operations of a refining process, according to embodiments of the disclosure.

### DETAILED DESCRIPTION

Referring now to the drawings in which like numerals indicate like parts throughout the several views, the following description is provided as an enabling teaching of exemplary embodiments, and those skilled in the relevant art will recognize that many changes can be made to the embodiments described. It also will be apparent that some of the desired benefits of the embodiments described may be obtained by selecting some of the features of the embodiments without utilizing other features. Accordingly, those skilled in the art will recognize that many modifications and adaptations to the embodiments described are possible and may even be desirable in certain circumstances. Thus, the following description is provided as illustrative of the principles of the embodiments and not in limitation thereof.

The phraseology and terminology used herein is for the purpose of description and should not be regarded as limiting. For example, terms such as “spectroscopic analyzer,” “analyzer,” and “spectrometer” may be used interchangeably. As used herein, the term “plurality” refers to two or more items or components. A multi-component sample may refer to a single (one) sample including a plurality of components, such as two or more components. The terms “comprising,” “including,” “carrying,” “having,” “containing,” and “involving,” whether in the written description or the claims and the like, are open-ended terms, e.g., to mean “including but not limited to,” unless otherwise stated. Thus, the use of such terms is meant to encompass the items listed thereafter, and equivalents thereof, as well as additional items. The transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not necessarily, by itself, connote any priority, precedence, or order of one claim element over another or the temporal order in which acts of a method are performed, but are used merely as labels to distinguish one claim element having a certain name from another element having a same name (but for use of the ordinal term) to distinguish claim elements.

Certain terminology used herein may have definitions provided for the purpose of illustration and not limitation. For example, as used herein, “sampling circuit” may refer to an assembly for facilitating separation of a sample of a material, a sample of a composition of material, and/or a sample of a product from a refining process, for example, for removal and/or analysis of the sample. Similarly, the term “sample conditioner” may refer to an assembly for facilitating preparation of a sample for analysis, for example, to improve the accuracy of analysis of the sample and/or to provide consistency and/or repeatability of the analysis of the sample or more than one sample. Additionally, the term “sample stream” may refer to a portion of a sample stream supplied to one or more spectroscopic analyzers for spectroscopic analysis by the one or more spectroscopic analyzers.

As used herein, the term “sample introducer” may refer to a component or assembly that may be used to facilitate the provision of a conditioned sample (portion or stream) to one or more spectroscopic analyzers for analysis. As used herein, the term “sample probe” may refer to a component or an interface used to facilitate collection of a sample for analysis by, for example, one or more spectroscopic analyzers. As used herein, the term “analyzer probe” may refer to a component of one or more spectroscopic analyzers that facilitates direction of electromagnetic radiation (e.g., light energy) from a source through a sample stream (e.g., a conditioned sample stream) to detect and/or measure one or more of absorbance, transmittance, transflectance, reflectance, attenuated total reflectance (ATR), or scattering intensity associated with the sample stream. As used herein, the term “sample cell” may refer to a receptacle or cell for receipt of samples for analysis or measurement, for example, by a spectroscopic analyzer.

As used herein, the term “spectroscopic analyzer” may refer an analyzer that may be used to measure or predict one or more properties of a sample of, for example, a material, a composition of materials, and/or a refinery product (an intermediate product or an end or final product). In some embodiments, the spectroscopic analyzers may be used on-line or in a laboratory setting. “Spectroscopic analyzer” may refer in some instances to a spectroscopic analyzer assembly, which may include a spectroscopic analyzer and an analyzer controller in communication with one or more spectroscopic analyzers. The analyzer controller may be configured for use with a corresponding spectroscopic analyzer for pre-processing and/or post-processing steps or procedures related to a spectroscopic analysis, as will be understood by those skilled in the art. In some embodiments, the analyzer controller may be physically connected to the spectroscopic analyzer. In some such embodiments, the spectroscopic analyzer may include a housing, and at least a portion of the analyzer controller may be contained in the housing. In some embodiments, the analyzer controller may be in communication with the spectroscopic analyzer via a hard-wired communications link and/or a wireless communications link. In some embodiments, the analyzer controller may be physically separated from the spectroscopic analyzer and may be in communication with the spectroscopic analyzer via a hard-wired communications link and/or a wireless communications link. In some embodiments, physical separation may include being spaced from one another, but within the same building, within the same facility (e.g., located at a common manufacturing facility, such as a refinery), or being spaced from one another geographically (e.g., anywhere in the world). In some physically separated embodiments, both the spectroscopic analyzer and the analyzer controller may be linked to a common communications network, such as a hard-wired communications network and/or a wireless communications network. Such communications links may operate according to any known hard-wired communications protocols and/or wireless communications protocols, as will be understood by those skilled in the art.

As used herein, the term “on-line” may refer to equipment and/or processes that are physically located at or adjacent to processing assemblies during operation and, for at least some embodiments, may be capable of providing real-time and/or near real-time analysis and/or data capable of real-time and/or near real-time analysis. For example, in some embodiments, an on-line spectroscopic analyzer may receive one or more sample streams directly from a processing assembly or process and analyze the one or more sample streams in real-time or near real-time to provide results that may, in some embodiments, be used to at least partially control operation of one or more processing assemblies and/or one or more processes in real-time or near real-time. In some embodiments, the on-line spectroscopic analyzer or analyzers may be physically located in a laboratory setting. This may be either extractive (e.g., a sample stream is drawn off of a processing unit and supplied to a spectroscopic analyzer and/or to one or more sensors) or in situ (e.g., a probe of a spectroscopic analyzer or one or more sensors is present in a conduit associated with the processing assembly).

As used herein, the term “at-line” may refer to equipment and/or processes that are physically located at or adjacent to processing assemblies during operation, but which, for at least some embodiments, are not capable of providing real-time and/or near real-time analysis and/or are not capable providing data capable of real-time and/or near real-time analysis. For example, in an “at-line” process, a “field analyzer” located physically at or adjacent to a processing assembly may be used to analyze a sample withdrawn from the processing assembly or process and manually taken to the field analyzer for analysis. In some embodiments, the at-line spectroscopic analyzer or analyzers may be physically located in a laboratory setting. For example, in some embodiments, an at-line spectroscopic analyzer would not receive a sample stream directly from processing assemblies, but instead, would manually receive a sample manually withdrawn from a processing unit by an operator and manually taken or delivered by the operator to the at-line spectroscopic analyzer.

Certain terminology used herein to describe a flow or material, such as a “feed,” a “stream,” a “feedstock,” “products,” and the like may be used interchangeably and are not meant to be limiting. A strict literal definition of these terms may not be appropriate as a standard definition because, for example, a characterization of a particular flow may change depending on the viewpoint perspective used within the refining process. In some embodiments, it may be understood that a flow referred to as a “product” produced by a certain refining process and/or processing unit, for example, may simultaneously be viewed as a “feedstock” input for another refining process and/or processing unit (e.g., a downstream refining process and/or downstream processing unit).

As used herein, the term “predicting” may refer to measuring, estimating, determining, and/or calculating one or more properties of a material, a composition of materials, and/or a refinery product (e.g., an intermediate product or an end or final product) based on, for example, a mathematical relationship, a correlation, an analytical model, and/or a statistical model.

A petroleum refining process may use one or more process units to divide, combine, or alter characteristics of various hydrocarbon feeds in the production of end products, performing processes, such as, for example, distillation, catalytic reforming, catalytic cracking, alkylation, isomerization, and other processes. The feed and products from these processes may provide, for example, gas (liquified petroleum gas), naphtha, aviation fuel, motor fuel, and other feedstocks for petrochemical industries. The prediction of the yields and quality of these components, however, may typically require detailed knowledge of the properties of the feedstock and input and output streams, including, for example, physical properties, chemical composition, the molecular weight of constituents, and/or other properties which often require laboratory analysis to obtain a result.

In some embodiments, spectroscopic analyzers may be used to non-invasively predict (or determine) properties and/or related information associated with materials associated with a petroleum refining operation. The systems and methods disclosed herein may use spectroscopic analyzers and/or other instrumentation to predict properties of hydrocarbons and other materials, and the properties may be used, either alone or in combination with additional information, to enhance operation or control of units and processes within a petroleum refining operation. For example, one or more spectroscopic analyzers may be linked to one or more signal processing devices to permit numerical treatment of spectra generated by the one or more spectroscopic analyzers, and, in at least some embodiments, the treated spectra may be communicated to one or more controllers configured to use the treated spectra to determine control signals for controlling one or more aspects of the distillation process and/or related refining processes. In some embodiments, the assemblies, systems, apparatuses, and processes may be used in, for example, an on-line manner for real-time data interpretation and may provide results with accuracy at least similar to that of other primary test methods, but in a comparatively more responsive manner. Non-on-line analysis is also contemplated.

In some refining operations, a raw petroleum stream (e.g., raw crude, raw shale oil, and/or other sources) may be supplied to a refinery and may be conveyed by one or more material pumps to refinery processing units for converting the raw petroleum into desired intermediate and final products. For example, the raw petroleum may be conveyed to refinery processing units, such as, for example, an atmospheric distillation unit, a vacuum distillation unit, a saturated gas unit, fracking oil fractionators, condensate fractionators, and/or other refinery processing units performing, for example, distillation/fractionation processes, treatment processes, thermal separation processes, hydrogen-treating processes, and/or cracking processes.

The content and/or properties of different supplies of the raw petroleum may vary significantly, depending on, for example, the supplier and/or even different batches from the same supplier. For example, a tank farm including multiple tanks containing respective supplies of petroleum having different characteristics may be positioned upstream of a fractionation process. For example, the respective supplies of petroleum may include petroleum containing light, heavy, sweet, sour, and/or other source stocks of varying weight, sulfur content, and hydrocarbon chain length. As a result, it may be desirable to subject the different supplies of raw petroleum to different processes and/or different processing conditions in order to improve efficiencies associated with operation of the refinery. Thus, determining the content and/or properties of the raw petroleum and/or the content and/or properties of the intermediate products or final products may be desirable in order to control one or more of the refinery processing units to promote more efficient operation of the refinery. For example, according to some embodiments, the raw petroleum (e.g., one or more hydrocarbon feedstocks), intermediate products, and/or final products may be analyzed to determine content and/or properties, and the results of the analysis may be used to at least partially control the refining operation, including, for example, distillation/fractionation processes, thereby to enhance the processes. In some embodiments, such analysis may be conducted in real-time, or near-real-time, for example, such that adjustments may be made to (1) parameters and/or properties associated with the raw petroleum, the intermediate materials, and/or the final products, and/or (2) parameters and/or operating characteristics associated with one or more of the refinery processing units, such as, for example, processing units associated with the distillation processes. In some embodiments, real-time, or near-real-time, analysis may result in more effective and/or more responsive adjustments being performed, thereby resulting in enhanced distillation processes, which may improve efficiencies associated with the refining operation. For example, real-time, or near-real-time, analysis of the raw petroleum supplied to the refinery may facilitate more effective control of various processes and/or prioritization of production, for example, at least in part in response to demand, production targets, and/or other factors.

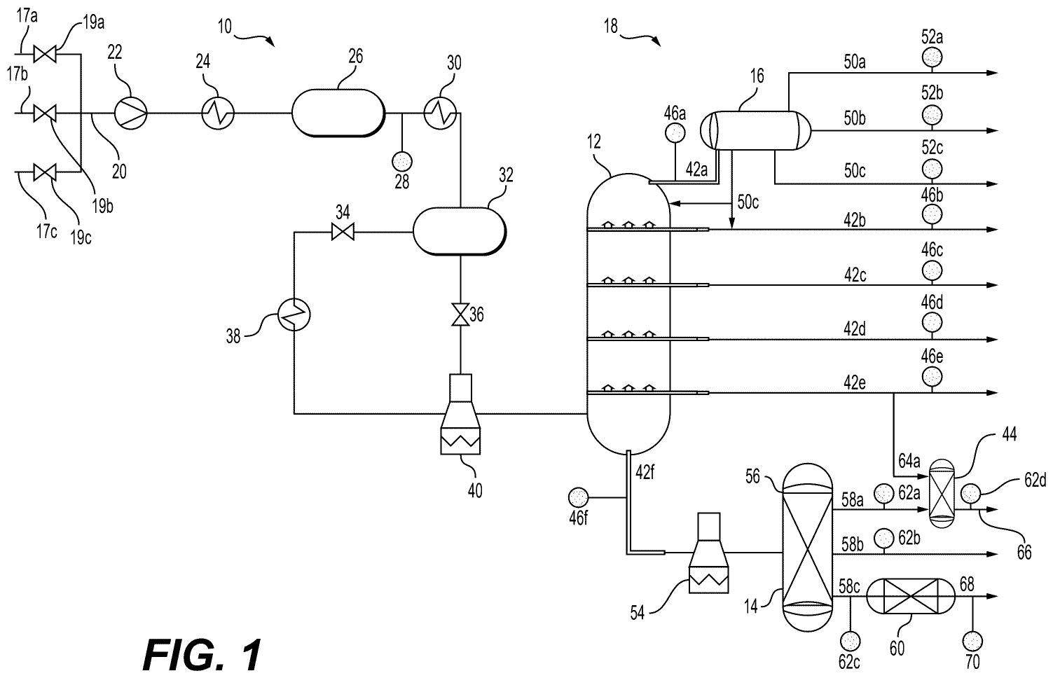

FIG. 1 is a schematic block diagram of an example refining operation 10 including example fractionation units used in the refining operation 10. The fractionation units may include, for example, an atmospheric distillation unit, a vacuum distillation unit, a fracking oil fractionation unit, a saturated gas unit, a condensate fractionation unit, and/or other processing units for separating a hydrocarbon feedstock into fractions. FIG. 1 shows an example atmospheric distillation unit 12, an example vacuum distillation unit 14, an example saturated gas unit 16, and example measurement devices (e.g., 28, 46a-46f, 52a-52c, 62a-62d, and 70) to facilitate control of the refining operation 10, according to embodiments of the disclosure. For example, as shown in FIG. 1, in some embodiments, the refining operation 10 may include a processing unit assembly 18 including one or more fractionation units (e.g., atmospheric distillation unit 12, vacuum distillation unit 14, and/or other processing units).

The refining operation 10 may be supplied with a hydrocarbon feedstock 20 (e.g., a petroleum feed) from one or more hydrocarbon sources positioned upstream of the refining operation 10. For example, as shown in FIG. 1, the hydrocarbon feedstock 20 may be a composite or blended hydrocarbon feedstock including a plurality of raw hydrocarbon feedstocks 17a, 17b, and/or 17c from respective hydrocarbon feedstock flows. A lesser or greater number of raw hydrocarbon feedstocks are contemplated. Valves 19a, 19b, and/or 19c may be used to control the mass/volumetric flow rates of the raw hydrocarbon feedstocks 17a, 17b, and/or 17c, for example, such that the hydrocarbon feedstock 20 may be a blend of certain characteristics based on objectives of the refining operation 10. For example, in some embodiments, the properties of the blend of the hydrocarbon feedstock 20 may be monitored and used to predict and/or tailor the yield of various products from downstream fractionation processes. In some embodiments, the properties of the blend may be monitored, and/or the feed rates of the raw hydrocarbon feedstocks 17a, 17b, and/or 17c may be controlled to target specific yields of various products from downstream fractionation processes.

The hydrocarbon feedstock 20 may be supplied via operation of a material pump 22 to be prepared for further refining operations in processing units, such as the atmospheric distillation unit 12, the vacuum distillation unit 14, the gas plant unit 16, and/or other processing units for performing, for example, treatment processes, thermal separation processes, hydrogen-treating processes, and/or cracking processes. Other processes are also contemplated. The hydrocarbon feedstock 20 may be conveyed to one or more raw crude preheat exchangers 24, after which it may be fed to a desalter 26. To determine properties and/or the contents of the hydrocarbon feedstock 20 material stream, at least one measurement device 28 may be positioned to analyze the stream between the desalter 26 and one or more desalted crude preheat exchangers 30. In some embodiments, the measurement device(s) 28 may include one or more spectroscopic analyzers for generating one or more spectra indicative of one or more properties and/or the content of the stream. From the desalted crude preheat exchangers 30, the hydrocarbon feedstock 20 may be fed to a preflash drum 32 where the stream may be directed, via operation of one or more valves 34 and/or 36, to a preflashed crude preheat exchanger 38 and to an atmospheric unit heater 40, or directly from the preflash drum 32 to the atmospheric unit heater 40. Alternately, or in addition, when the contents of the hydrocarbon feedstock 20 from the hydrocarbon source contain a proportion of lighter components where the pressure required to suppress vaporization is higher than desired, the hydrocarbon feedstock 20 may be directed from the preflash drum 32 to the atmospheric distillation unit 12. At least some of the raw crude preheat exchangers 24, the desalted crude preheat exchangers 30, the preflashed crude preheat exchanger 38, and the atmospheric unit heater 40 may use waste process heat from other parts of the refining operation 10.

In some embodiments, the measurement device(s) 28 may include a first spectroscopic analyzer, which may be used to predict properties of the material in the hydrocarbon feedstock 20 and/or the content of the hydrocarbon feedstock 20 (e.g., components of the stream). Measurements of the measurement device(s) 28 may be used to assist with and/or facilitate control at least a portion of the distillation process.

As shown in FIG. 1, the hydrocarbon feedstock 20 may pass from the atmospheric unit heater 40 to the atmospheric distillation unit 12, which may include an atmospheric distillation column. The atmospheric distillation unit 12 may include, for example, a tower or column for performing a fractional distillation process that divides the petroleum material of the of the hydrocarbon feedstock 20 from the hydrocarbon source (e.g., a composite petroleum feedstock) into separate hydrocarbon fractions. In some embodiments, the atmospheric distillation unit 12 may be configured perform an atmospheric distillation process on the petroleum material. Such a process may be complex, involving multiple separations in one or more different columns and/or one or more portions of columns. In some embodiments, the hydrocarbon feedstock 20 may include two or more fraction components. In some embodiments, the hydrocarbon feedstock 20 may include two or more fraction components. Heat supplied, for example, by the atmospheric unit heater 40 prior to introduction to the atmospheric distillation unit 12, may be used in the atmospheric distillation process to separate two or more fractions of the hydrocarbon feedstock 20 from one another and/or the rest of the stream.

For example, as shown in FIG. 1, within the atmospheric distillation unit 12, a fractional distillation process may result in separation of the stream into a plurality of product cuts or product fractions 42a, 42b, 42c, 42d, 42e, and 42f (or more), which may each correspond to respective ranges of boiling points. As shown in FIG. 1, relatively lighter fractions of the separated stream may include a gaseous product 42a, and the gaseous product 42a may be withdrawn from the atmospheric distillation unit 12 as an intermediate “overhead” product. In some embodiments, the refining operation 10 may include a saturated gas unit 16 to which the gaseous product 42a may be supplied. In some embodiments, the gaseous product 42a may also be flared.

As shown in FIG. 1, in some embodiments, the separation into fractions may result in additional distillation, such as, for example, products 42b through 42e, and one or more of the other distillation products 42b through 42e may be withdrawn at from the atmospheric distillation unit 12 at a plurality of respective locations along the height of the atmospheric distillation unit 12. For example, distillation products 42b through 42e may be removed at locations along the height of the column of the atmospheric distillation unit 12. In some embodiments, one or more of the other distillation products 42b through 42e may be routed to (1) other processing units of the refining operation 10 for further processing at one or more processing units downstream of the atmospheric distillation unit 12, (2) to local storage, and/or (3) to pipeline(s) for supply to off-site locations.

For example, the distillation product 42b may include Light Straight Run (LSRN), for example, light naphtha, which may be supplied to Light Ends Processing (LEP). The distillation product 42c may include Heavy Straight Run (HSRN), for example, heavy naphtha, which may be supplied to a downstream second processing unit, such as a reformer unit. The distillation product 42d may include light gas oils (LGO), such as jet fuel and/or kerosine, which may be supplied to other processing units, such as, for example, a Merox treatment unit for the removal of mercaptans and/or supplied to storage. In some embodiments, diesel oil may be separated from the distillation product 42d and supplied to storage. In some embodiments, the distillation product 42e may include, for example, heavy gas oils (HGO), which may be supplied to a downstream second processing unit, such as a fluid catalytic cracking (FCC) unit 44, for example, as shown in FIG. 1. The distillation product 42f may include, for example, a bottoms stream including residuum and non-volatile components of the hydrocarbon feedstock 20, which may be supplied to, for example, a vacuum distillation unit 14. In some embodiments, residuum may be used to produce, for example, a coker feed, asphalt, heavy lubricating oils, and/or waxes.

In some embodiments, as shown in FIG. 1, one or more of the distillation products of the atmospheric distillation unit 12 may be analyzed, for example, by one or more respective measurement devices 46a, 46b, 46c, 46d, 46e, and 46f (or more), which may include spectroscopic analyzers. In some embodiments, the one or more measurement devices 46a through 46f may be configured to receive samples for analysis from one or more flows of the distillation products 42a through 42f, for example, as described herein, in an on-line manner and/or in-line manner. In some embodiments, the one or more measurement devices 46a through 46f may be configured to provide a real-time analysis of one or more of the distillation products 42a through 42f, which may result in more responsive control and/or more effective control of the distillation process, which may be tailored to, for example, prioritization of production in response to demand, production targets, and/or other factors.

In some embodiments, properties of the distillation products may be estimated based at least in part on composition and/or properties of the hydrocarbon feedstock 20 received by the refining operation 10. The composition and/or properties may be determined and/or achieved by, for example, changing the proportions of raw hydrocarbon feedstocks (e.g., 17a, 17b, and/or 17c shown in FIG. 1) mixed to produce the hydrocarbon feedstock 20. By determining the compositions and/or properties of the hydrocarbon feedstock 20, the volume/mass percent yield of fractionated rundown streams (e.g., the one or more distillation products of the atmospheric distillation unit 12) may be estimated. In some embodiments, “properties” may include, for example, any characteristics of the material, the content of the material (e.g., material yield and/or ratios of different types of material within a material containing more than a single type of material), physical conditions of the material, such as temperature, density, pressure, and/or flow rate of the material. Other properties of the material or materials are contemplated, such as, for example, properties and/or characteristics that are known to those skilled in the art.

For example, in some embodiments, properties of the distillation products may be estimated based at least in part on properties of the hydrocarbon feedstock 20 received by the refining operation 10. For example, boiling points of one or more of the distillation products may be estimated based at least in part on estimated and/or predicted properties of the hydrocarbon feedstock 20 received by the refining operation 10. Based at least on part on the estimated boiling points, the yields of one or more of the distillation products (e.g., unit materials produced by the one or more fractionation units (e.g., light, medium, and/or heavy fractions)) may be estimated based at least in part on the estimated and/or predicted properties of the hydrocarbon feedstock 20. For example, light naphtha and heavy naphtha, distillation products that may be useful for a gasoline blending process, may have boiling points ranging from about 50 degrees Fahrenheit (F) to about 300 degrees F., or ranging from about 50 degrees F. to about 400 degrees F. Light gas oils, such as jet fuel and/or kerosine, may have boiling points ranging from about 400 degrees F. to about 600 degrees F. HGO may have boiling points ranging from about 600 degrees F. to about 800 degrees F. Residuum and other bottoms may have boiling points over about 950 degrees F. It will be appreciated that the boiling points and ranges described herein are approximate, and the actual properties of light, medium, and heavy fractions may vary significantly, for example, for hydrocarbon materials of different supplies and/or hydrocarbon materials sourced from different suppliers. In some embodiments, heat for the atmospheric distillation unit 12 (e.g., for heating recycling streams and/or other processes in the atmospheric distillation unit 12) may be supplied to the atmospheric column (e.g., for the column) by, for example, a fired reboiler, circulating steam, and/or other heat sources.

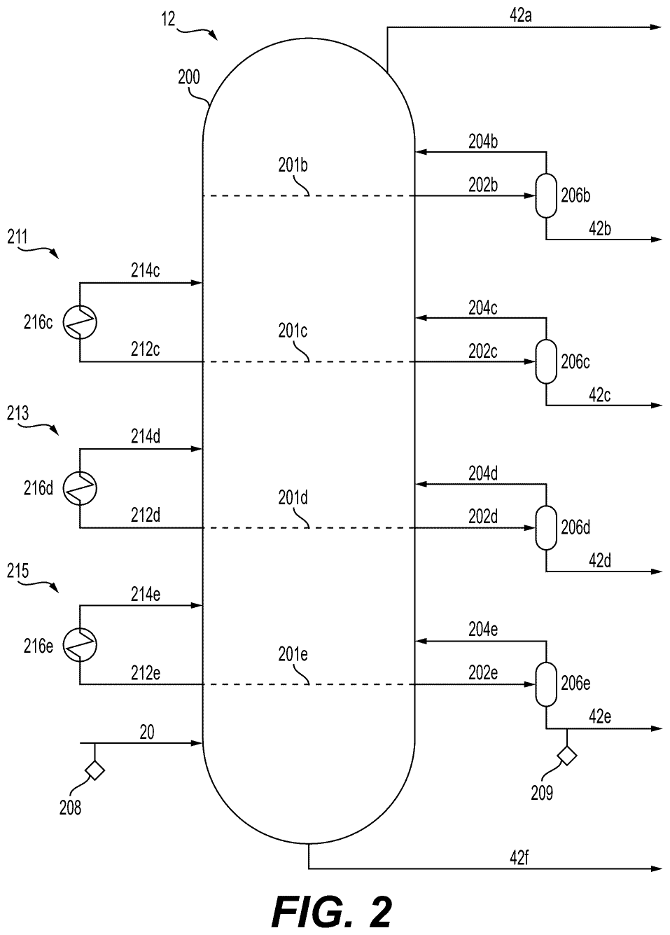

FIG. 2 is a schematic diagram of an example atmospheric distillation unit 12 and associated hydrocarbon feeds, according to embodiments of the disclosure. In some embodiments, as shown in FIG. 2, the atmospheric distillation unit 12 may include an atmospheric distillation processing unit. The atmospheric distillation unit 12 may include an atmospheric column 200, and the interior of the atmospheric column 200 may be provided with a plurality of vertically spaced trays 201b, 201c, 201d, and 201e (or more) (e.g., bubble trays), each configured to provide a vapor/liquid separation interface at discrete heights of the atmospheric column 200. Although four example bubble trays are shown in FIG. 2, any type, spacing, and/or number of trays may be used. The atmospheric distillation unit 12 may be configured to withdraw various distillation products, such as, for example, light naphtha, heavy naphtha, kerosene, light gas oil, heavy has oil, and/or other distillation products.

As shown in FIG. 2, for example, a stream of heavy gas oil 202e may be withdrawn via a conduit providing a flow path to a stripper 206e, where the stream of heavy gas oil 202e may be separated into (a) an overhead component 204e that may be recirculated back to a portion of the atmospheric column 200 and (b) a product stream 102e that may be withdrawn via conduit as unit materials and/or intermediate materials. The stripper 206e may be configured to separate liquid and vapor components of the stream of heavy gas oil 202e via one or more processes, such as a flash to atmospheric pressure, a vacuum, the addition of stripping steam, and/or other processes. In some embodiments, the atmospheric distillation unit 12 may include one or more additional strippers 206b, 206c, and 206d (or more), each of which may operate in a manner at least similar to the manner in which the stripper 206a operates, to separate other side products from the column 200, for example, some or all of distillate streams 202b, 202c, 202d, and 202e (or more) into respective overhead and product streams.

In some embodiments, one or more of the strippers 206b through 206d may not discriminate by the phase of the stream and may instead redirect at least a portion of the distillate streams 202b through 202e in the form of respective overhead recycle streams 204b, 204c, 204d, and 204e (or more) back to the atmospheric column 200, or pass on the full stream as distillation products 102b through 102e, for example, without recycling and/or redirecting any of distillate streams 202b through 202e. In some embodiments, the atmospheric distillation unit 12 may have no strippers, or a lesser or greater number of strippers, than those shown in FIG. 2.

In some embodiments, the atmospheric distillation unit 12 may include one or more pump-around circuits 211, 213, and/or 215, for example, to withdraw liquid streams from the column 200 and/or recycle heat. The liquid streams may be reintroduced by the pump-around circuits 211, 213, and/or 215 to a different location in the column 200. The reintroduction of the liquid streams to different locations in the column 200 may provide additional operational control over the yield of one or more fractions of the distillation process. The pump around circuits 211, 213, and/or 215 may form at least a portion of fluid circuits for one or more of the strippers 206b through 206d, and/or may operate independently of the strippers 206b through 206d, for example, on separate fluid circuits. One or more of the pump-around circuits 211, 213, and/or 215 may include heat exchangers 216c, 216d, 216d, and 216e (or more). Heat exchangers 216c through 216e may be configured to remove heat to from one or more recycle streams 212c, 212d, and 212e (or more). Heat removed from the distillation process in the column 200 by heat exchangers 216c through 216e may be used in other portions of the refining operation. For example, a recycle product corresponding to heavy gas oil in recycle stream 212e may be withdrawn and supplied to the heat exchanger 216e for the removal of heat, and following the transfer of thermal energy, a cooled recycle stream 214e may be reintroduced at a relatively higher point into the atmospheric column 200. In some embodiments, the heat exchangers 216c and 216d may operate in a similar fashion for respective recycle streams 212c and 212d, if desired.

In some embodiments, the heat exchangers 216a, 216b, and/or 216c in the pump around circuits 211, 213, and/or 215 may use a coolant (e.g., cooling water and/or air) as a medium of exchange. The heat removed from the one or more recycle streams 212c, 212d, and/or 212e may be used in other processes, such as, for example, heating the hydrocarbon feedstock 20 via the raw crude preheat exchanger 24, the desalted crude preheat exchanger 30, the preflashed crude preheat exchanger 38, and/or the atmospheric unit heater 40.

Referring again to FIG. 1, in some embodiments, a fractionation unit (e.g., vacuum distillation unit 14) may be used to perform a vacuum distillation process in the refining operations to produce, for example, lube oil fractions, FCC feedstocks of low carbon content, and/or other products. Residuum, atmospheric bottoms, and other non-volatile components separated by an atmospheric distillation process may be removed from the atmospheric column 200 (e.g., distillation products 42f in FIG. 1 and FIG. 2) and used as a feedstock for the vacuum distillation unit 14. In some embodiments, distillation products 42f may be composed of the heaviest of the cuts of distillation products from the atmospheric distillation unit 12, and may consist of some or all of the components from the hydrocarbon feedstock that have true boiling points above approximately 650 degrees F. at atmospheric pressure. For example, the contents of the distillation products 42f may have true boiling points ranging from approximately 650 degrees F. to 800 degrees F. Alternatively, the contents of the distillation products 42f may have true boiling points ranging from approximately 800 degrees F. to approximately 1,025 degrees F. at atmospheric pressure.

The contents of distillation products 42f may be determined prior to the vacuum distillation process by one or more measurement devices 46f, which may include one or more spectroscopic analyzers positioned to receive a sample along the path between the atmospheric distillation unit 12 and the vacuum distillation unit 14. The measurement device(s) 46f may, for example, be used to determine asphaltene concentration and/or other properties of the distillation products 42f. In some embodiments, the asphaltene concentration may be used, for example, to control parameters of the vacuum distillation unit 14 (e.g., throughput and/or the amount of heat added to vacuum distillation unit 14 by the vacuum unit heater 54).

As shown in FIG. 1, the distillation products 42f may be heated in a vacuum unit heater 54 to prepare the distillation products 42f as a feedstock for a vacuum distillation unit 14. In some embodiments, partial quenching or other processes may also be used to separate lighter components from the distillation products 42f, for example, to reduce coking in the vacuum distillation unit 14 and/or other downstream second processing unit(s).

In some embodiments, the vacuum distillation unit 14 may include a vacuum column 56 configured to receive the distillation products 42f as a feedstock stream. Vacuum pumps, steam ejectors, and/or other processes may be used to draw a vacuum inside the vacuum column 56. The distillation process in the vacuum distillation unit 140 may separate, in the vacuum, components of the distillation products 42f into fractions. In some embodiments, process parameters such as throughput, temperature, steam additives, and/or internal vacuum pressure in the vacuum distillation unit 14 may be used to control the vacuum distillation process for efficiency and/or to optimize the products, for example, based at least in part on value of the products.

The intermediate and/or unit material products produced by the vacuum distillation unit 14 may include, for example, vacuum distillation products 58a, 58b, and/or 58c that may be used as feeds for numerous other downstream refining processes and/or processing units that may produce, for example, asphalt, residual fuel oil, #6 fuel oil, marine Bunker C fuel oil, and/or other products. In some embodiments, light gas fractions that are not useful in vacuum distillation products 58a through 58c may be removed from the vacuum column 56 as tail gas overhead and recycled back to the vacuum unit heater 54.

In some embodiments, vacuum distillation products 58a may include, for example, light vacuum gas oil (LVGO) and heavy vacuum gas oil (HVGO) that may be used as feedstock for one or more second processing units downstream, such as FCC unit 44. In some embodiments, the HVGO also may undergo a reduced crude conversion (RCC) process and/or other processes to produce products for, for example, use in the FCC unit 44. Contents and properties of the vacuum distillation products 58a may be analyzed and predicted by one or more measurement devices 62a, which may include, for example, one or more spectroscopic analyzers positioned to receive samples from vacuum distillation products 58a.

Distillate fuel oils, cylinder stock, and/or other cylinder oils may make up at least a part of the vacuum distillation products 58b removed from the vacuum column 56. Contents and properties of the vacuum distillation products 58a may be analyzed and predicted by one or more measurement device(s) 62b, which may include, for example, one or more spectroscopic analyzers positioned to receive samples from vacuum distillation products 58b.

Vacuum distillation products 58c removed from the vacuum column 56 of the vacuum distillation unit 14 may include vacuum residuum and/or bottoms, which may be used in one or more second processing units downstream. Contents and properties of the vacuum distillation products 58c, such as, for example, asphaltene content, may be analyzed and predicted by one or more measurement devices 62c, which include, for example, one or more spectroscopic analyzers positioned to receive a sample from along the path between the vacuum distillation unit 10 and the one or more of the second processing unit(s). Alternatively, sample(s) of the vacuum distillation products 58c may be analyzed in a laboratory to determine the contents and properties. In some embodiments, at least a portion of the vacuum residuum and/or bottoms in vacuum distillation products 58c may be directed to one or more of the second processing unit(s), such as solvent deasphalter unit 60 and/or a visbreaker unit. In some embodiments, at least a portion of the vacuum residuum and/or bottoms in vacuum distillation products 58c may provide feedstock for, for example, an asphalt oxidizer unit or mixed with other fuel oils. In some embodiments, the properties and/or content of the vacuum distillation products 58c predicted by measurement device 62c may be used to control operations of the vacuum distillation unit 14 and/or second processing units positioned downstream, for example, according to properties, such as the asphaltene content, alkane solvent-to-vacuum bottoms ratio, and/or process parameters, such as temperature, flow rate, and/or vacuum pressure in the vacuum column 56. In some embodiments, the properties may be predicted by analyzing the feedstocks supplied to the fractionation units (e.g., the atmospheric distillation unit(s) 12 and/or the vacuum distillation unit(s) 14).

Referring again to FIG. 1, in some embodiments, the refining operation 10 may include a saturated gas unit 16 configured to separate light fractions of the distillation products 42a from the atmospheric column 200 of the atmospheric distillation unit 12 into liquid components and vapor components. For example, as shown in FIG. 1, the light fractions of the distillation products 42a may be supplied from the atmospheric distillation unit 12 as “overhead” products 42a to the saturated gas unit 16, for example, via one or more of conduits, piping, and/or tubing. Contents and properties of the distillation products 42a supplied as a feedstock to the saturated gas unit 16 may be analyzed and predicted by one or more measurement devices 46a, which include one or more spectroscopic analyzers positioned to receive samples from distillation products 42a.

In some embodiments, at least a portion of the saturated gas unit 16 may include a separator for separating the distillation products 42a into liquid components and vapor components. For example, in some embodiments, the distillation products 42a may be supplied to a condenser and/or an accumulator, and liquid components 48 may be separated and returned to the atmospheric distillation unit 12 as a recycle stream and/or may be supplied to the distillation products 42b, which may include light naphtha.

In some embodiments, gaseous components of the distillation products 42a may be separated via the saturated gas unit 16 into multiple unit materials as intermediate gas products 50a, 50b, and/or 50c, for example, for storage, use as feedstocks for second processing units positioned downstream, and/or distributed as finished products. For example, (a) the intermediate gas products 50a may include C3/C4 fractions and/or other liquified petroleum gas (LPG), (b) the intermediate products 50b may include products for one or more polymerization processes and/or and alkylation processes, and (c) the intermediate products 50c may include ethers and other components, which may be used for gasoline blending processes and/or may be used for production of petrochemicals and other products.

In some embodiments, one or more of the intermediate gas products 50a through 50c from the saturated gas unit 16 may be analyzed via one or more of the measurement devices 52a through 52c, respectively. The one or more measurement devices 52a through 52c may include one or more spectroscopic analyzers configured to analyze and predict the properties and/or content of one or more of the intermediate gas products 50a through 50c. The properties and/or content of the intermediate gas products 50a through 50c may be compared with, for example, respective target properties and/or content (and/or target ranges of properties and/or content) of products produced by the saturated gas unit 16. Alternatively, or in addition, the properties and/or content of the intermediate gas products 50a through 50c may be compared with respective target properties and/or content (and/or target ranges of properties and/or content) of feedstocks for one or more second processing units positioned downstream.

The properties and/or content, distribution, and/or use of the distillation products from the fractionation units (e.g., 42a through 42f, 50a through 50c, and 58a through 58c in FIG. 1) may vary based on the capabilities, layout, and/or desired final products of different refineries or refining operations 10. For example, the one or more second processing unit(s) described herein (e.g., for example, a reformer unit, a Merox treatment unit, an FCC unit, a deasphalter unit, and/or other processing units) are examples, and the assemblies, systems, apparatuses, and processes of this disclosure are thus not meant to be construed as limited to refineries and/or refining operations with those capabilities and/or processing units. For example, in some embodiments, the refining operation 10 may not include a saturated gas unit 16, and light products 42 from atmospheric distillation may be transported and/or sold for further use or off-site processing. In some embodiments, the refinery or refining operations 10 may include one of a particular second processing unit, a plurality of a particular second processing units, or none of a particular second processing unit. For example, the refinery or refining process may include one deasphalter unit 60 downstream of the atmospheric distillation unit 14, a plurality of downstream deasphalter units, or no downstream deasphalter units. Similarly, the refinery or refining process may include one FCC unit 44 downstream of the atmospheric distillation unit 12 and/or vacuum distillation unit 14, a plurality of downstream FCC units, or no downstream FCC units.

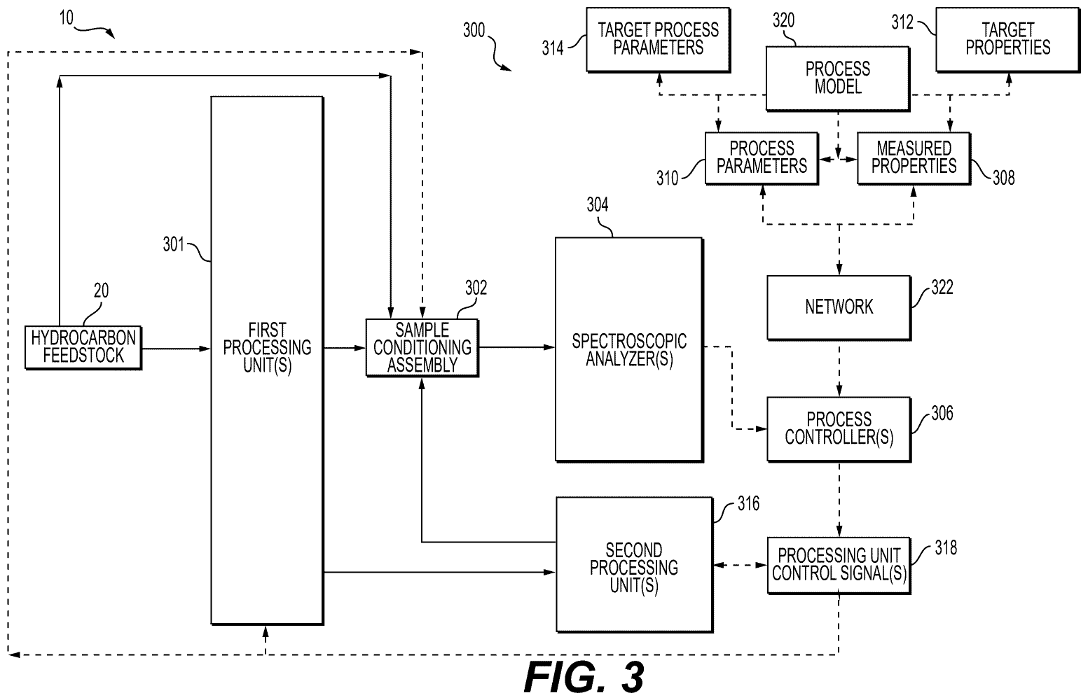

It will also be appreciated that, in addition to spectroscopic analysis, other devices and/or processes for determining parameters, such as temperatures, pressures, and/or flow rates may be provided for the various feeds and products of a fractionation or distillation process for the purpose of monitoring and controlling the process. For example, temperature sensors may be used to sense temperature the various streams and convert the temperatures into electrical signals for use by one or more process controllers (see, e.g., FIG. 3). In some embodiments, the electrical signals for monitoring pressures, flow rates, and/or other parameters may be communicated to the process controllers in the same or similar fashion as those of the one or more temperature signals, for example, via known wired communication protocols and/or known wireless communication protocols.

As shown in FIG. 2, other sensors, such as sensors 208, may be positioned to monitor the hydrocarbon feedstock 20 entering the atmospheric column 200. Sensor 209, for example, may be positioned to monitor one or more of the distillation products 42a and/or recycle streams 212c, 212d and/or 212e, distillation products 42b, 42c, 42d, and/or 42e after stripping, residuum/bottoms in distillation products 42f directed for vacuum distillation, and/or at other positions. The electrical signals for monitoring pressures, flow rates, and/or other process parameters may be communicated to the process controllers in the same or similar fashion as those of the one or more temperature signals.

It will also be understood that the positions and/or uses of spectroscopic analyzers, sensors, other measurement devices, and/or control elements disclosed herein are provided as examples only, and other positions and/or uses are also contemplated. For example, spectroscopic analyzers 28, 46a, 46b, 46c, 46d, 46e, 46f, 52a, 52b, 52c, 62a, 62b, 62c, and/or 70 are not limited to the positions schematically depicted in FIG. 1, and their respective uses are not limited to monitoring and predicting specific examples of the product properties and process parameters described herein. In addition, a lesser or greater number of analyzers and/or other measurement devices may be used than those described and/or shown in the accompanying figures. Factors such as end product selection, refinery-specific layout, and/or refinery capabilities may be considered when selecting the application of spectroscopic analyzers and/or other measurement devices for controlling and/or enhancing the refining operation 10.

In some embodiments, the one or more spectroscopic analyzers, may facilitate such improvement or optimization on-line during operations of one or more refining processes (e.g., in real-time) and/or processing unit assemblies, which may reduce or eliminate inefficient operations and control delays.

In some embodiments, using spectroscopic analyzers, a sample of material may be fed to one or more spectroscopic analyzers for analysis, and a beam of electromagnetic radiation may be transmitted into the material sample, resulting in the spectroscopic analyzer measuring a spectral response representative of the chemical composition of the sample material, which may be used to predict (or determine) content and/or properties of the sample material via the use of modeling. The spectral response may include a spectrum related to the absorbance, transmission, transflectance, reflectance, attenuated total reflectance (ATR), and/or scattering intensity caused by the material sample over a range of wavelengths, wavenumbers, or frequencies of the electromagnetic radiation.

Spectroscopic analyzers may be of the portable/field type, laboratory type, and/or process (continuous on-line, in-line) type. Material to be sampled may flow substantially intermittently or continuously past a point from where the material for sampling may be systematically collected or drawn for measurement by the spectroscopic analyzers. The resulting data may thereafter be processed, such as, for example, by taking the first or higher order derivative, and/or by applying statistical techniques (e.g., a least-squares regression, a multiple linear regression (MLR), a partial least squares regression (PLS), a principal component regression (PCR), and/or other methods) to provide an output signal indicative of the concentration of the particular component of the material sample. In some embodiments, a single spectroscopic analyzer may be used to alternate between a feed stream and a product stream of a processing unit. In some embodiments, multiple spectroscopic analyzers may be used to analyze a feed stream and a product stream.

In some embodiments, the spectroscopic analyzers may be calibrated to generate standardized spectral responses. The spectroscopic analyzers may be calibrated using calibration data derived from one or more of a set of known reference fuels, a library of known spectral data, and/or an analytical model of the alkylation process. For example, using the standardized spectral responses, the calibration of the one or more spectroscopic analyzers may be updated prior to, or periodically during, on-line operations. Example methods for determining and using the standardized spectral responses are described in, for example, U.S. Pat. No. 11,702,600, titled “Methods and Assemblies for Determining and Using Standardized Spectral Responses for Calibration of Spectroscopic Analyzers,” herein incorporated by reference in its entirety. The methods and assemblies in the above-referenced application may be used to calibrate or recalibrate a spectroscopic analyzer when the spectroscopic analyzer changes from a first state to a second state, for example, the second state being defined as a period of time after a change to the spectroscopic analyzer causing a need to calibrate the spectroscopic analyzer. In some embodiments, the recalibration may result in the spectroscopic analyzer outputting a standardized spectrum, for example, such that the spectroscopic analyzer outputs a corrected material spectrum for an analyzed material, including one or more of an absorption-corrected spectrum, a transmittance-corrected spectrum, a transflectance-corrected spectrum, a reflectance-corrected spectrum, an attenuated total reflectance (ATR)-corrected spectrum, or an intensity-corrected spectrum and defining the standardized spectrum.

In some embodiments, the systems and methods disclosed herein may also be used to control and optimize petroleum refining operations off-line, such as remotely in a laboratory setting. These resources may be used, for example, in situations where a sample handling and/or preparation procedure may not be supported by on-line operations.

FIG. 3 shows a distillation unit control assembly 300 to enhance control of a refining process associated with a petroleum refining operation (e.g., refining operation 10). For example, the refining operation 10 may include one or more first processing unit(s) 301 including one or more fractionation units (e.g., an atmospheric distillation unit 12, a vacuum distillation unit 14, a saturated gas unit 16, a fracking oil fractionation unit, a condensate fractionation unit, and/or other processing units). In some embodiments, the refining operation 10 may also include one or more of fracking oil fractionators, condensate fractionators, and/or other refinery processing units. In some embodiments, the distillation unit control assembly 300 may include a sample conditioning assembly 302 configured to condition material samples for analysis, one or more spectroscopic analyzer(s) 304 configured to provide material sample spectra during operation of one or more processing units associated with the refining operation 10, and/or one or more process controller(s) 306 in communication with the spectroscopic analyzers 304 and configured to control operations (e.g., prescriptively control operations) of at least some of the processing units of the refining operation 10.

In some embodiments, the refining operation 10 may also include one or more second processing unit(s) 316 arranged downstream of the one or more first processing unit(s) 301. For example, the one or more second processing units 316 may include a hydrotreater unit, a deasphalter unit, a visbreaker unit, an FCC unit, a solvent processing unit, thermal processing unit, and/or other processing units, as described herein, for processing the intermediate unit materials produced by the first processing unit(s) 301 into forms appropriate for use by the second processing units 316.

As schematically shown in FIG. 3, the distillation unit control assembly 300 may be configured to use the one or more process controller(s) 306 in communication with the one or more spectroscopic analyzers 304 and may be configured to prescriptively control (e.g., semi-autonomously, autonomously, and/or fully control) at least a portion of the refining operation 10. As shown in FIG. 3, solid lines generally indicate hydrocarbon material feedstock flows and/or hydrocarbon product flows, which may be used to supply one or more unit materials (e.g., including one or more of intermediate materials and/or unit product materials) for analysis. Dashed lines generally indicate sample analysis, control signals, and/or control system flows.

In some embodiments, the spectroscopic analyzer(s) 304 of the distillation unit control assembly 300 may be configured to generate one or more spectra indicative of properties and/or contents of samples of material input flows (e.g., the hydrocarbon feedstock 20) and/or one or more unit materials produced by the one or more first processing unit(s) 301 and/or the second processing unit(s) 316. In some embodiments, one or more of a vacuum distillation unit 14 or a saturated gas unit 16 may be considered as second processing units 316 downstream of an atmospheric distillation unit 12 or other first processing unit(s) 301.

In some embodiments, the one or more sample properties and/or sample contents generated from analyzing a sample with the spectroscopic analyzer(s) 304 may include a content ratio indicative of relative amounts of one or more hydrocarbon classes present in the sample material (e.g., a hydrocarbon feedstock sample, a unit material sample, and/or another material sample). One or more of the spectroscopic analyzers(s) 304 may include, for example, one or more near-infrared (NIR) spectroscopic analyzers, one or more mid-infrared (mid-IR) spectroscopic analyzers, a combination of one or more NIR and mid-IR spectroscopic analyzers, one or more Raman spectroscopic analyzers, or one or more nuclear magnetic resonance (NMR) spectroscopic analyzers. In some embodiments, one or more of the spectroscopic analyzer(s) 304 may be reinforced and/or hardened for use in an on-line analyzing process, and in some embodiments, one or more of the spectroscopic analyzer(s) 304 may be at least partially housed in a temperature-controlled and/or damage-resistant cabinet. In some embodiments, a photometer with present optical filters moving successively into position, may be used as a type of spectroscopic analyzer.

In some embodiments, a fiber optic probe in communication with one or more of the spectroscopic analyzer(s) 304 may be inserted directly into a conduit or conduits containing a flow or stream of the material to be analyzed to facilitate analysis, for example, by preventing a need to extract a sample of the material for analysis from a separate conduit or a sample collection system.

As shown in FIG. 3, in some embodiments, the one or more process controllers(s) 306 of the distillation unit control assembly 300 may be configured to control one or more operating parameters against operating constraints associated with one or more processing units 301 and/or 316 associated with the refining operation 10. For example, as described herein, each of the one or more spectroscopic analyzer(s) 304 may output a signal or signals communicated to the one or more process controller(s) 306, which may be configured to mathematically manipulate the signal(s) (e.g., take a first or higher order derivative of the signal and apply statistical techniques, such as a least-squares regression, a multiple linear regression (MLR), a partial least squares regression (PLS), a principal component regression (PCR), a Gauss-Jordan row reduction, a multiple linear regression, and/or other methods) received from the spectroscopic analyzer(s) 304, and subject the manipulated signal(s) to an analytical model configured to generate material properties and/or parameters of interest, for example, as described herein. In some embodiments, such analytical models may be derived from signals obtained from spectroscopic analyzer measurement of the one or more unit materials (e.g., the intermediates and/or distillation process products).

In some embodiments, the one or more process controllers 306 may be configured to receive one or more measured properties 308 associated with a material, and use the one or more measured properties 308 associated with the material to determine changes that may be made to one or more process parameters 310 of the refining operation 10 that may cause the refining process to achieve material outputs that more accurately and converge on one or more of the target properties 312 (e.g., target contents and/or target material characteristics/properties). In some embodiments, the one or more process controllers 306 may be configured to receive signals indicative of one or more measured properties 308 associated with a material product from the one or more spectroscopic analyzer(s) 304, and use the one or more measured properties 308 associated with the material product to identify determine changes that may be made to one or more process parameters 310 of the refining operation 10 that may cause the refining process to achieve material outputs that more accurately and converge on one or more of the target properties 312 (e.g., target contents and/or target material characteristics/properties).

In some embodiments, the one or more process controller(s) 306 may be configured to combine multiple control strategies. For example, in some embodiments, one or more measured properties 308 of the hydrocarbon feedstock 20 may be determined using data (e.g., in signal form) from the measurement device(s) 28 (e.g., spectroscopic analyzer 28 in FIG. 1) and used to determine whether adjustments to the feed temperature should be made in order to achieve certain material fractions (and/or the properties of certain fractions) from the atmospheric distillation unit 12. Feed temperature may be adjusted, for example, via receipt of heat from the raw crude preheat exchanger 24, the desalted crude preheat exchanger 30, and/or the preflashed crude preheat exchanger 38. Alternatively, or in addition, in some embodiments, one or more temperature correction factors may be applied to, for example, one or more of the hydrocarbon feedstock sample spectra, one or more of the hydrocarbon feedstock sample properties, one or more of the unit material sample spectra, or one or more of the unit material properties. The correction factor(s) may be derived, for example, from literature, historical operational data, empirical data, one or more analytical models related to the refining process(es), and/or other sources.

In some embodiments, property value data from the measurement device(s) 28 may be used adjust the feed ratio(s) and/or feed space velocity of the hydrocarbon feedstock 20, for example, via control of the material pump 22. In some embodiments, asphaltene concentrations of the hydrocarbon feedstock 20 may be predicted after desalting, and measured properties 308 associated with the desalter crude water used with the desalter 26 may be used to control operation of the desalter 26. For example, noting any differences between the analyzed asphaltene content and a desired content, the one or more process controller(s) 306 may be configured to send one or more signals to one or more of the heaters (e.g., preheat exchangers 24, 30, and/or heaters associated with a downstream deasphalter or visbreaker) to increase or decrease the respective temperatures of one or more of the heaters (or, as described, applying one or more known temperature correction factors), which may change the “severity” of downstream processes, such as in the atmospheric column 200 of the atmospheric distillation unit 12. In some embodiments, the one or more signals may be used to control the severity of the vacuum distillation unit(s) 14, for example, by adjusting one or more of temperature, throughput, cut point, or unit space velocity.

One or more measurement devices may also be used to determine the contents of raw tank fills upstream of the atmospheric distillation unit 12 (e.g., for changes in the source(s) of the hydrocarbon feedstock 20), the contents of overhead effluent water from the atmospheric distillation unit 12 to evaluate performance of the desalter 26, and/or in support of periodic refinery mass balancing in the estimation of accounted losses and unaccounted losses during operation of the atmospheric distillation unit 12 and other processes of the refining operation 10.

For example, referring to FIG. 3 and operation of the distillation unit control assembly 300, the one or more process controllers 306 may be configured to generate one or more processing unit control signals 318, which may be configured to control (e.g., prescriptively control) one or more of the processing units of the refining operation 10. For example, one or more signals may be used for controlling parameters of the hydrocarbon feedstock 20 supplied to the atmospheric distillation unit 12, such as, for example, temperature, feed ratios, and/or feed space velocities. In some embodiments, the processing unit control signal(s) 318 may include, for example, a first signal representative of the temperature of the hydrocarbon feedstock 20 supplied to a processing unit (for example, hydrocarbon feedstock 20 entering the fractional distillation column 200 of the atmospheric distillation unit 12). The properties and/or contents of the hydrocarbon feedstock 20 may be analyzed by a first spectroscopic analyzer and one or more signal representative of the one or more hydrocarbon feedstock 20 properties and/or contents may be received by one or more of the process controller(s) 306. During the fractional distillation process in the atmospheric distillation unit 12, fractions separated in the column 200 (e.g., 42a through 42f) may have measured properties predicted by a one or more additional spectroscopic analyzer(s) 304 and additional signals may be communicated to the one or more process controller(s) 306 (e.g., based at least in part on the sample spectra from the spectroscopic analyzer(s)). The process controller(s) 306 may be configured to determine one or more differences between (a) measured properties 308 for a fraction (or fractions) produced and (b) a set of target properties 312 for the fraction or fractions. The one or more differences may be determined, for example, via comparison between the measured properties 308 and those determined from one or more analytical models representative of the distillation process (e.g., a process model 320). In some embodiments, the process model 320 may be a model representative of a specific process such as, for example, a fractional distillation model for an atmospheric distillation process. In some embodiments, process model 320 may be a collection of analytical models representative of the one or more portions of the refining operation 10 (e.g., a large portion of (or the entire) the refining operation 10). Determining the differences, via the one or more process controllers 306, may be based at least in part on the process model 320, and/or a set of predetermined target properties 312. The one or more process controller(s) 306 may thereafter use at least some of the differences to adjust one or more of the set of process parameters 310, for example, to reduce the one or more differences and/or to cause one or more of the products to converge on the target properties 312 or within a range of the target properties 312.

The one or more process controller(s) 306 may be configured to execute or perform the process model 320 (e.g., an analytical fractional distillation model) to improve the accuracy and/or efficiency of one or more of, for example: (a) predicting one or more hydrocarbon feedstock parameters of a hydrocarbon feedstock 20 supplied to the one or more first processing units 301; (b) predicting one or more intermediate properties associated with intermediate materials produced by the one or more first processing units 301; (c) controlling the one or more hydrocarbon feedstock parameters of the hydrocarbon feedstock 20 supplied to the one or more first processing units 301; (d) controlling the one or more properties associated with the intermediate materials produced by the one or more first processing units 301; and/or (e) the predicting one or more target properties of the downstream materials produced by one or more of the second processing units 316.

In some embodiments, the process model 320 may include a fractional distillation model, which, in some embodiments, may be and/or include a machine-learning-trained model. In some embodiments, the process model 320 may include a fractional distillation model and/or may be and/or include an artificial intelligence (AI) model. In at least some such embodiments, the process controller(s) 306 may be configured to: provide, to the fractional distillation model, refining and/or fractional distillation processing data related to: (a) material data including one or more of (i) feedstock data indicative of one or more parameters and/or properties associated with the hydrocarbon feedstock 20, (ii) unit material data indicative of one or more unit material properties associated with the one or more unit materials, and/or (iii) downstream material data indicative of one or more downstream material properties associated with one or more downstream materials produced by the one or more second processing units 316; and/or (b) processing unit data including (i) first processing unit 301 data indicative of one or more operating parameters (e.g., process parameters 310) associated with operation of the one or more fractionation units of the one or more first processing units 301, (ii) second processing unit 316 data indicative of one or more operating parameters (e.g., process parameters 310) associated with operation of the one or more of the second processing units 316, and/or (iii) sample conditioning assembly data 302 indicative of operation of a sample conditioning assembly 302. Alternatively, or in addition, the process controller(s) 306 may be configured to: (a) prescriptively control, based at least in part on fractional distillation process data: (ii) one or more hydrocarbon feedstock parameters, properties, and/or content associated with the hydrocarbon feedstock 20, (iii) one or more first operating parameters (e.g., process parameters 310) associated with operation of the one or more first processing units 301, (iv) one or more properties associated with the one or more unit materials, (v) properties and/or content of the one or more unit materials, (vi) one or more intermediates properties and/or contents associated with the one or more intermediate materials; (vii) one or more second operating parameters associated with operation of the one or more second processing units 316 positioned downstream relative to the one or more first processing units 301; and/or (viii) one or more downstream properties associated with the one or more downstream materials produced by the one or more second processing units 316.

In some embodiments, one or more sample properties predicted or determined by the spectroscopic analyzer(s) 304, operating parameters of the processing units 301 and/or 316, and other information may be output to an output device. The output device may include, for example, a display in communication with at least one of the one or more spectroscopic analyzer(s) 304, process controller(s) 306, and/or processing units 301 and/or 316 engaged in the refining process. In some embodiments, the output device may be configured to update the sample properties and/or operating parameters in real-time and may include visual alarms and/or audio alarms associated with target or threshold values for the sample properties or operating parameters.

In some embodiments, the one or more process models 320 (e.g., the fractional distillation model) may include one or more distillation algorithms (e.g., in support of, as part of, or in addition to fractional distillation data for a fractional distillation model). The distillation algorithms may be configured to determine, based at least in part on data from the fractional distillation process, target properties 312 for one or more of the hydrocarbon feedstock 20, unit materials, or downstream materials, for example, as described herein. In some embodiments, the distillation algorithms further may be configured to control operation (e.g., prescriptively control operation) of one or more of the first processing units 301 and/or second processing units 316, for example, to produce one or more of unit materials having unit material properties within a predetermined range of target properties 312 for the unit materials, and/or one or more of downstream materials having downstream materials properties within a predetermined range of target properties 312 for the downstream materials. In some embodiments, the respective ranges may include values within a range above (but not below) the target unit material properties 312 of the unit materials and/or the target properties 312 of the downstream materials, within a range below (but not above) the target properties 312 of the unit materials and/or the target properties 312 of the downstream materials, or within a range surrounding (on either or both sides of) the target properties 312 of the unit materials and/or the target properties 312 of the downstream materials. The distillation algorithms may be configured to determine one or more measured properties 308 and/or content of the unit materials produced by the one or more first processing units 301 and/or one or more measured properties 308 and/or content of the downstream materials produced by the one or more second processing units 316, for example, as described herein. In some examples, the content of downstream materials may be controlled and adjusted through control of one or more of the properties and/or content of the hydrocarbon feedstock 20 supplied to one or more of the first processing units 301, the properties and/or content of the intermediate materials produced by the one or more first processing units 301, the properties and/or content of the unit product materials, or operational control of the first processing units 301 and/or second processing units 316.

In some embodiments, the one or more process controller(s) 306 may be configured to execute or process the one or more distillation algorithms of the process model 320, for example, to determine one or more differences between the measured properties 308 and respective target properties 312 and/or contents. For example, the distillation algorithms may be configured to determine one or more of: (a) unit material differences between (i) the actual measured properties 308 of the unit materials and (ii) the target properties 312 of the unit materials, or (b) downstream material differences between (i) the actual measured properties 308 of the downstream materials and (ii) the target properties 312 of the downstream materials. In some embodiments, the one or more process models 320, for example, via the one or more process controller(s) 306, may be configured to change (e.g., update), based at least in part on one or more of the unit material differences or the downstream material differences, the one or more distillation algorithms to adjust the one or more of the unit material differences or the downstream material differences to achieve material outputs that more accurately and responsively converge on the target properties 312 and/or contents. The process model 320 may, in some embodiments, use the one or more distillation algorithms, in some examples, to actively or dynamically adapt (e.g., in real time) on-line to material differences between the measured properties 308 and the target properties 312.

In some embodiments, one or more of the spectroscopic analyzer(s) 304 may be configured to analyze, for example, during the fractional distillation process, one or more downstream properties and/or contents associated with downstream materials produced by the one or more second processing units 316 positioned downstream of the first processing units 301 (e.g., the atmospheric distillation unit 12, the vacuum distillation unit 14, the saturated gas unit 16, and/or other processing units). The one or more process controller(s) 306 may be configured to receive one or more signals representative of the one or more downstream properties and/or contents and determine, based at least in part on the one or more process models 320 and the one or more signals, one or more differences between (a) a set of actual downstream properties and/or contents for materials produced by the one or more second processing units 316 and (b) a set of target downstream properties and/or contents for materials produced by the one or more second processing units 316. The one or more process controller(s) 306 may be configured to thereafter, based at least in part on the one or more differences, adjust one or more of the set of actual downstream process parameters 310 of the one or more second processing units 316, for example, to reduce the one or more differences. In some embodiments, the one or more process controller(s) 306 may be configured to set target process parameters 314, for example, to cause the fractional distillation process to produce downstream materials with measured properties 308 and/or contents to converge on target properties 312 and/or target contents and/or within a range of the target properties 312 and/or target contents.

In some embodiments, the process controller(s) 306 may be configured to control (e.g., prescriptively control), for example, during the refining process, based at least in part on one or more hydrocarbon feedstock parameters, one or more hydrocarbon feedstock sample properties, and/or the one or more unit material sample properties: (a) the one or more hydrocarbon feedstock parameters associated with the hydrocarbon feedstock 20 supplied to the one or more first processing unit(s) 301; (b) one or more intermediates properties associated with the intermediate materials produced by one or more of the first processing unit(s) 301; (c) operation of the one or more first processing units 301; (d) one or more unit materials properties associated with the one or more unit product materials; and/or (e) operation of one or more second processing unit(s) 316 positioned downstream relative to the one or more first processing unit(s) 301. The one or more second processing unit(s) 316 may include, for example, an FCC unit 44 configured separate FCC effluent and other gasoline blending products from feedstocks received from one or more of the first processing unit(s) 301. Alternatively, or in addition, one or more second processing units positioned downstream may include, for example, a deasphalter unit 60 positioned for the separation and removal of asphaltenes, resins, and/or metals from vacuum residue and very heavy vacuum gas oils produced by a vacuum distillation unit 14. Other second processing unit(s) may also be anticipated.

In some embodiments, the one or more unit material sample properties includes ethane content, propane content, propene content, isobutane content, or n-butane content.

The one or more process controller(s) 306 may be configured to communicate one or more processing unit control signals 318 configured to adjust process parameters 310 associated with processing units performing one or more refining processes. In some embodiments, a single process controller 306 may be in communication with all processing units to be controlled. In some embodiments, a system controller for the refining operation 10 may be configured to communicate with individual process controllers 306 associated with each processing unit. Alternatively, or in addition, each processing unit may have a dedicated process controller 306 to control process parameters 310 related to the process or processes performed by that particular processing unit.

The one or more processing unit control signal(s) 318 may include, for example, one or more signals indicative process parameters 310 such as: (a) a rate of supply or feed ratio of a feedstock to one or more of the processing units; (b) the pressure of a feedstock to one or more of the processing units; (c) a preheating temperature of a feedstock to one or more of the processing units; (d) a temperature in the atmospheric column 200 of an atmospheric distillation unit 12 and/or the vacuum column 56 of a vacuum distillation unit 14; (e) or a pressure associated with one or more of the processing units. Control of other process parameters 310 associated with operation of one or more of the processing units is also contemplated. In some embodiments, controlling the one or more process parameters 310 of the one or more of the processing units may include controlling the one or more process parameters 310 against operating constraints associated with one or more of the processing units.

In some embodiments, the control described herein (e.g., prescriptive control) may result in at least a portion of one or more refining processes to produce one or more of: (a) one or more intermediate materials each having one or more properties within a selected range of one or more target properties 312 of the one or more intermediate materials; (b) one or more unit materials each having one or more properties within a selected range of one or more target properties 312 of the one or more unit materials; or (c) one or more downstream materials each having one or more properties within a selected range of one or more target properties 312 of the one or more downstream materials. In some embodiments, the one or more unit materials may include one or more of intermediate materials or unit product materials produced by for example, the one or more first processing unit(s) 301. In some embodiments, the control may result in causing a refining process to achieve material outputs that more accurately and responsively converge on one or more of the target properties 312. In some embodiments, the control may also result in optimizing one or more target properties 312 of one or more unit materials, and/or one or more target properties 312 of one or more downstream materials produced by the one or more second processing unit(s). The control may thereby, for example, enhance the performance and/or the accuracy of one or more of the first processing unit(s) 301, the second processing unit(s) 316, and/or other processing units to achieve material outputs that more accurately and responsively converge on one or more of the target properties 312 or within a range of target properties 312.

In some embodiments, at least some portions of the process model 320 used in the prescriptive control may be hosted separately, such as, for example, on an independent computer system, server, and/or a network 322. In some embodiments, the full process model 320 used in the control may be integrated and hosted commonly on one or more computer systems (not shown), for example, on the same network 322. Alternatively, or in addition, at least a portion of the process model 320 may be represented by a common set of processor instructions and/or a common set of algorithms stored on a memory device.

Aspects of the subject matter described herein may also be practiced on, or in conjunction with, other computer system configurations beyond those described herein, including, for example, multiprocessor systems, microprocessor-based or programmable consumer electronics, minicomputers, mainframe computers, handheld computers, mobile telephone devices, tablet computing devices, special-purposed hardware devices, network appliances, and the like. Some or all of the computer system configurations may be connected to the network 322.

In some embodiments, the one or more process controller(s) 306 may be configured to communicate one or more of sample properties, unit material sample properties, downstream material sample properties, or other properties to simulation software to model one or more processing unit material yields and/or unit material characteristics. In some embodiments, the simulation software may be configured to determine, based at least in part on the provided properties, one or more process parameters 310 to achieve target process parameters for one or more processing units and/or target properties of one or more material samples (e.g., unit materials, downstream materials, and/or other materials). In some embodiments, the simulation software may be integrated into, or run in parallel with, the one or more process models 320. In some embodiments, the simulation software may be configured to determine one or more properties of the one or more downstream materials or materials configured to be finished product materials based at least in part on one or more properties of the hydrocarbon feedstock 20 supplied to fractionation unit(s) of the first processing units 301 and/or the one or more process parameters 310 indicative of operation conditions of one or more first processing units 301 and/or the one or more second processing units 316.

At least some embodiments may include a sample conditioning assembly 302 configured to condition material samples from one or more processes in the refining operation 10, including, for example, fractionation processes and/or distillation processes, to enhance the analysis provided by one or more spectroscopic analyzers 304. For example, for an on-line spectroscopic analyzer, an intermittent sample stream or a substantially continuous sample stream may be subjected to a process (e.g., conditioning) when preparing a representative sample for analysis by a spectroscopic analyzer. The conditioning process may be implemented using the sample conditioning assembly 302 described herein, as well as components for fast loop and/or pretreatment operations. The function of these components may enhance results of analysis performed by one or more spectroscopic analyzers. For example, a sample substantially free of contaminates, includes particulates and/or fluids such as water, and/or maintained within specified temperature ranges, may result in more accurate measurements performed by the one or more spectroscopic analyzer(s) 304.

In some embodiments, for example, the distillation unit control assembly 300 may include a sample conditioning assembly 302 configured to condition a material sample or material samples of a process feedstock and/or product, for example, prior to being supplied to the one or more spectroscopic analyzer(s) 304. In some embodiments, the sample conditioning assembly 302 may be configured to filter the samples, change (e.g., control) the temperature of the samples, degas the samples, and/or dilute the samples in solvent. In some embodiments, the sample conditioning assembly 302 also may be configured to condition samples of the unit materials, for example, prior to being supplied to the one or more spectroscopic analyzer(s) 304, to filter the samples of the unit materials, to change (e.g., control) the temperature of the samples of the unit materials, degas the samples of the unit materials, and/or dilute in solvent the samples of the unit materials. In some embodiments, sample conditioning by the sample conditioning assembly 302 may result in more accurate, more repeatable, and/or more consistent analysis of the samples by the one or more spectroscopic analyzers(s) 304. In some embodiments, the one or more process controller(s) 306 may be configured to control (e.g., prescriptively control at least some aspects of operation of the sample conditioning assembly 302, for example, as described herein.

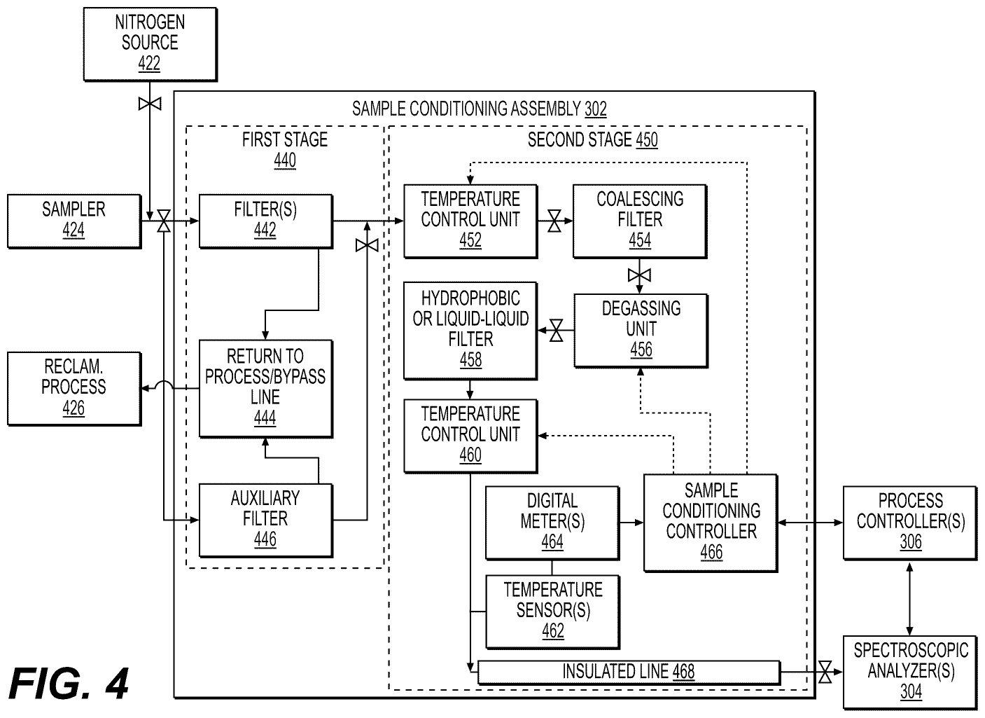

FIG. 4 is a schematic block diagram of an example sample conditioning assembly 302, according to embodiments of the disclosure. As shown in FIG. 4, the sample conditioning assembly 302 may include a sampler 424 configured to extract a material sample from the sample stream from a point or points associated with a fractionation and/or distillation process (e.g., before, during, and/or after the fractionation and/or distillation process). The material sample may be in the form of an intermittent sample stream and/or a substantially continuous sample stream. The sampler 424 of the sample conditioning assembly 302 may include a sample probe, a sample supply pump, and/or a varying header pressure from a header configured to provide a consistent flow of the sample stream to the sample conditioning assembly 302. The sample conditioning assembly 302 may include an enclosure for housing at least some of the components associated with the sample conditioning assembly 302. In some embodiments, the sample conditioning assembly 302 may include subsystems for performing processes such as, for example, water and contaminant filtration, temperature control, degassing treatment, and/or dilution.

For example, in some embodiments, a desired vapor pressure of a unit product material sample may be defined by a predetermined range (e.g., within a certain range of pounds per square inch (psi)). A degasser may therefore be provided in the sample conditioning assembly 302 to provide a degassing treatment to a unit product material sample having a relatively higher vapor pressure than the predetermined range, for example, to remove gases or bubbles from the sample.

In some embodiments, as shown in FIG. 4, the sample conditioning assembly 302 may include a first stage 440 and a second stage 450. Some embodiments may include only a single stage, and some embodiments may include more than two stages. The first stage 440 may include a first set of one or more filters 442 including a plurality of membranes configured to filter the extracted sample to thereby reduce the amount of particulates and water from the extracted sample. The first stage 440 may further include an auxiliary filter 446 in fluid communication with the sampler 424 and connected in parallel to the first set of one or more filters 442 for use during a maintenance of the first set of one or more filters 442. The auxiliary filter 446 may be configured to receive the extracted sample from the sampler 424. Whichever is in use, the first set of filters 442 or the auxiliary filter 446, may produce a filtered sample, which may include a substantially continuous sample stream. The removed particulates and water may be routed to a reclamation process 426, a sample recovery assembly, or a pump, for example, via a bypass line 444 configured to route the reduced particulate and water.

In some embodiments, at least a part of the sample conditioning assembly 302 may also be flushed out with a flow of compressed gas, such as nitrogen from a nitrogen source 422 and/or air to remove particulates in the sample conditioning assembly 302, thereby allowing for service and/or maintenance of the sample conditioning assembly 302. For example, when at least the first set of one or more filters 442 is being flushed and cleaned, for example, with the flow of compressed gas, samples may be directed to the auxiliary filter 446.

The second stage 450 of the sample conditioning assembly 302 may include a first temperature control unit 452. The first temperature control unit 452 may include one or more of a chiller, a heater, or other heat exchanging components in fluid communication with the first set of one or more filters 442 positioned to receive the filtered sample and change a temperature of the filtered sample to a predetermined temperature, which may render it more suitable for sample analyses. A sample having a regulated temperature may thus be supplied to the spectroscopic analyzer(s) 304, which may yield relatively more accurate analysis. The first temperature control unit 452 may heat or chill the sample to the predetermined temperature, for example, to provide a temperature-adjusted sample having a temperature within a first preselected temperature range of a target sample temperature. The second stage 450 may also include a degassing unit 456 configured to degas the filtered sample (e.g., a temperature-adjusted degassed sample).

The second stage 450 of the two-stage sample conditioning process may continue with a process for removing water or moisture from the sample via a second set of one or more filters. The second set of one or more filters may include a coalescing device, such as a coalescing filter 454, for example. The coalescing filter 454 may be configured to remove or reduce water and/or eliminate any liquid contaminants and/or mist based on one or more of three methods: (1) direct impact for particles having a particle size being approximately 1 micron or larger, (2) interception of particles having a particle size ranging from approximately 0.1 and 0.6 microns, and/or (3) diffusion for particles having a particle size approximately 0.1 micron or smaller. The water and/or moisture that has been removed by the coalescing filter 454 may be routed to the bypass line 444. In some embodiments, for example, the second set of filters in the second stage 450 may be configured to remove the remaining water and/or moisture from the sample, for example, using a hydrophobic filter 458 and/or a liquid-liquid membrane filter. For example, the hydrophobic filter 458 may be configured to prevent passage of remaining water and/or moisture in the sample. The hydrophobic filter 258 may include a known hydrophobic filter, such as a hydrophobic polytetrafluoroethylene (PTFE) membrane filter. Hydrophobic PTFE membrane filters may include a thin, highly porous film that behaves as an absolute retentive membrane. In some embodiments, the second stage 450 of the two-stage conditioning assembly process may further have the coalescing filter 454 positioned after the hydrophobic filter 458 or liquid-liquid membrane filter.

In some embodiments, the second stage 450 of the sample conditioning assembly 302 may further include one or more thermometers or temperature sensors 462 in communication with one or both of the first temperature control unit 452 and the second temperature control unit 460, and configured to generate one or more temperature signals indicative of the temperature of one or more of the filtered sample, the temperature-adjusted sample, the degassed sample, or the temperature-adjusted degassed sample. The sample conditioning assembly 302 may additionally include one or more digital meters 464 configured to monitor and/or display the measured temperature of the filtered sample. The second stage 450 may also include a second temperature control unit 460 in fluid communication with one or more of the degassing unit 456 or the second set of one or more filters and positioned to change a temperature of the degassed sample to provide a temperature-adjusted degassed sample, such that the temperature-adjusted degassed sample has a temperature within a second preselected temperature range for more accurate property analysis via the one or more spectroscopic analyzer(s) 304.

The second stage 450 of the sample conditioning assembly 302 may further include a sample introducer in fluid communication with the first temperature control unit 452 and the second temperature control unit 460 via an insulated sample line 468. The sample introducer may also be connected to the spectroscopic analyzer(s) 304 via the insulated sample line 468 to supply the conditioned sample to the spectroscopic analyzer(s) 304. In some embodiments, the first temperature control unit 452 (and the associated chillers, heaters, and/or other components) may also be connected to the sample introducer via an optical fiber cable configured to maintain the conditioned sample at a predetermined temperature before reaching the spectroscopic analyzer(s) 304. The predetermined temperature may be, for example, between 70° F. and 72° F. Other temperature ranges for the predetermined temperature are also be contemplated, depending on the characteristics and/or type of the spectroscopic analyzer(s) 304.

In some embodiments, the second stage 450 of the sample conditioning assembly 302 may further include a sample conditioning controller 466 in communication with the temperature sensors 462. The sample conditioning controller 466 may include a processor and a memory configured to store the measured temperature of the conditioned sample as temperature data in a data structure in the memory associated with the controller 466. The stored temperature data may be transmitted to a monitoring server via a cable and/or a network and may also be shared with one or more process controller(s) 306 associated with the distillation unit control assembly 300.

In some embodiments, a sample to be analyzed may not be conditioned, or it may not be possible or advantageous to adjust, for example, the temperature or vapor pressure of a sample to be analyzed. As an alternative, one or more correction factors may be applied to the sample spectra generated by the spectroscopic analyzer(s) 304 or applied by the one or more process controller(s) 306. The correction factor(s) may be derived from literature, historical operational data, empirical data, the one or more process models 320, and/or other sources.

Following conditioning by the sample conditioning assembly 302, the spectroscopic analyzer(s) 304 may use the reflected signal from a beam to determine a spectral response representative of the chemical composition of a material sample, for example, as described herein. The one or more spectra of the conditioned samples may be generated at one or more wavenumbers or wavelength bands (wavenumber=1/wavelength). The generation of the spectra may include receiving a selected number of spectra from a conditioned sample. The generation of the spectra may also include determining an average spectrum based on the received selected number of spectra of the conditioned sample stream.

Samples to be conditioned may be drawn from a variety of streams feeding from, and/or produced by, processing units during refining operations, as discussed herein. In some embodiments, for example, as shown in FIG. 1, the atmospheric distillation unit 12 may include an assembly having an atmospheric column 200 for separating an input hydrocarbon feedstock 20 into unit material fractions (e.g., material fractions 42a through 42f), including overhead gas, light naphtha, heavy naphtha, light gas oil, heavy gas oil, and/or residuum/bottoms. The vacuum distillation unit 14 may include an assembly having a vacuum unit heater 54 or furnace, a vacuum column 56, and a vacuum producing system including vacuum pumps, steam ejectors, and/or other components for drawing a vacuum. An incoming feedstock (e.g., distillation products 42f) to the vacuum distillation unit 14 may be separated into distillation products (e.g., distillation products 58a through 58c), including, for example, vacuum gas oils LVGO and HVGO, distillate fuel oils, cylinder stock, and/or vacuum residue/bottoms. The saturated gas unit 16 may include an assembly positioned to separate a feedstock (e.g., distillation products 42a) into products 50a through 50c. Each of these respective feedstocks and unit material products may have samples analyzed by spectroscopic analyzers, and the samples may be conditioned by the sample conditioning assembly 302 prior to the analysis. The signals received from the analysis may be used in feedback control and/or feedforward control, for example, to enhance control of different processes in the refining operation 10.

In some embodiments, advanced process control (APC)-related and/or machine-learning techniques for a refining process that includes fractionation units may include receiving, during a first time period of refining process, a first portion of a sample stream from a point in the continuous refining operation (e.g., one or more intermediate or final products produced) having a plurality of components and properties. The first portion of the sample stream may be analyzed by one or more spectroscopic analyzers, which may generate one or more spectra from the first portion of the sample stream at one or more wavelength bands to determine the composition of the sample. Iteratively, at successive time periods of the refining process, the APC-related and/or machine-learning techniques may include using the one or more spectroscopic analyzers to predict the properties of a portion of the sample stream from the sampling point in the refining process. In some embodiments, one or more process controllers may be configured to compare and control properties, amounts, and/or process parameters according to a set of predetermined targets for the properties, amounts, and/or process parameters associated with the refining process. Control of process parameters may include, for example, adjusting processing unit parameters such as cut point, temperature, throughput, feed ratio, unit space velocity, purity, concentration, and/or other parameters.

The iterative process of control may continue at successive time periods during the refining process, for example, until (a) deviations between (i) the predicted properties, amounts, and/or process parameters and (ii) the predetermined targets, approach zero, and/or (b) the predicted properties, amounts, and/or process parameters do not fall outside respective predetermined desired target ranges (e.g., target ranges for the one or more intermediate or final products produced). In some embodiments, the predicted properties, amounts, and/or process parameters may be controlled via the one or more process controllers to remain within respective predetermined ranges of product specifications (e.g., within maximums and/or minimums). The target ranges and product specification maximums and minimums may include, for example, predetermined specific refinery generating targets, process model predictions, marketing specifications, industry standard specifications, and/or other target characteristics.

In some embodiments, one or more process models 320 (e.g., one or more analytical and/or kinetic models) may be used to predict (or determine) process yields as a function of, for example, feedstock quality (e.g., content and/or properties of a feedstock supplied to a process or processing unit) and/or processing conditions or parameters. In some embodiments, the one or more process models 320 used in the controlling may include machine-learning capabilities (e.g., the ability to be self-trained, for example, on past, current, and/or future operational data), for example, such that the one or more process models 320 are periodically, intermittently, and/or continuously updated and/or improved. In some embodiments, a process model 320 may be configured to incorporate target process parameters 314 known to produce desired yields of certain fractions or products. In some embodiments, the one or more process models 320 may include target properties 312 and/or target contents for samples of specific material streams from which the one or more process controller(s) 306 may adjust or tune process parameters 310 to enhance the yield of desirable products (e.g., aromatics and isoparaffins for gasoline blending). In some embodiments, an optimizer algorithm may be incorporated into or used with the one or more process models 320 to determine an improved or optimum combination material properties or process parameters for the current conditions and update the target material properties and/or target process parameters used by one or more process controller(s) 306 accordingly.

Process parameters for enhanced control of a fractional distillation process and/or downstream processes may include, for example, but are not limited to, crude blend parameters to maximize C5 production, make-up water, desalter severity, stripping steam parameters, as well as temperatures, pressures, draw rates, and wash rates for light and heavy naphtha, kerosine, LGO, HGO, absorber temperatures, recycled lean oil flow rate and temperature, high pressure separator temperatures, and/or stripper reboiler duty. Other control parameters are also contemplated, such as controlling, based at least in part on the one or more of the ethane content, the propane content, the propene content, the isobutane content, or the n-butane content, one or more of: absorber pressure, lean oil flow rate, lean oil temperature, high-pressure separator temperature, reactor conversion, or stripper reboiler duty.

The control (e.g., prescriptive control) of the processing units (e.g., one or more first processing units 301 and/or one or more second processing units 316 downstream of the first processing units 301) may include comparing the measurements of sample properties from the spectroscopic analyzers 304 against predefined data sets. The data sets may be part of, or used by, the one or more process models 320. For example, the sample properties may be compared with sample properties from a material database, with one or more threshold values associated with operation of the processing units, and/or with predetermined target properties and/or content for the sample properties. In some embodiments, the predetermined target contents may be associated with one or more of: (a) the content of the hydrocarbon feedstock supplied to the one or more first processing units 301; (b) the target content of the intermediate materials produced by one or more of the first processing units 301; (c) the target content of the unit product materials produced by one or more of the first processing units 301; or (d) the target content of the downstream materials produced by one or more of the second processing units 316.

In some embodiments, an atmospheric distillation unit 12 may be a relatively high throughput processing unit, and a refining operation may include two, three, or more atmospheric distillation trains (e.g., operating in parallel), and therefore the operation and control of the atmospheric distillation unit(s) may be a relative priority as related to APC. For example, spectroscopic analyzers configured to predict composition and property information from the hydrocarbon feedstock 20 at a suitable data rate may provide the process controller(s) 306 accurate product quality and yield data related to the cuts/fractions (e.g., unit materials including distillation products 42a through 42f) from the atmospheric distillation process for improving and optimizing the process. In addition, or alternatively, the prediction and/or control may reduce the likelihood or prevent yield losses of high-value distillation products owing to pump-around control (e.g., FIG. 2) and/or poor heat-balance within the atmospheric distillation column 200.

For example, in some embodiments, one or more spectroscopic analyzers 304 may be used to collect spectra of samples of the hydrocarbon feedstock 20 supplied to, for example, the example atmospheric distillation unit shown in FIG. 1 and FIG. 2. For example, the one or more properties and/or contents may be correlated to traditional laboratory tests (e.g., performed via one or more primary test methods), including, for example, HPLC Heavy Distillate Analyzer (HDA) results for aromatic core type (e.g., 1-ring core, 2-ring core, 3-ring core, 4-ring core, and/or polars), or ASTM D2887 high temperature simulated distillation. In some embodiments, the data from the laboratory tests and/or simulated distillation may be included in the one or more process models 320. The one or more properties and/or contents may include, for example, elemental content, API gravity, UOP K factor (e.g., associated with gas oil for an FCC unit), distillation points, coker gas oil content, carbon residue content, nitrogen content, sulfur content, saturates content, thiophene content, single-ring aromatics content, dual-ring aromatics content, triple-ring aromatics content, and/or quad-ring aromatics content.

In some embodiments, properties of the hydrocarbon feedstock 20 may also be analyzed after a switch in refinery supply from a current hydrocarbon source to a different hydrocarbon source. Characterization by, for example, true boiling point (TBP) may allow control during switches in the hydrocarbon source of the hydrocarbon feedstock 20 to identify expected changes in yield for the fractions of the separated hydrocarbon feedstock 20 (e.g., distillation products 42a through 42f) from the fractional distillation processes. For example, yield fractions associated with one or more unit materials from atmospheric distillation may be determined based at least in part on predicted boiling points (and/or other properties or characteristics) from the samples of the hydrocarbon feedstock 20. In some examples, the samples of the unit materials may include unit materials that may at least partially contain naphtha, and the predicted boiling points may be used for controlling one or more of: (a) downstream flows of intermediate materials or unit product materials associated with the naphtha; (b) downstream flows of intermediate materials or unit product materials associated with distillates; (c) the ratio of downstream flows associated with the naphtha to downstream flows associated with kerosene; or (d) operation of one or more naphtha strippers.

In some embodiments, at least a portion of the fractional distillation process in the atmospheric column 200 of the atmospheric distillation unit 12 may be controlled based at least in part on process parameters, such as, for example, temperature, for example, by adjusting the heat added via one or more of the upstream heaters and/or preheat exchangers, such as those shown in FIG. 1 (e.g., via the raw crude preheat exchanger 24, the desalted crude preheat exchanger 30, the preflashed crude preheat exchanger 38, and/or the atmospheric unit heater 40). For example, operating under relatively more severe distillation conditions (e.g., at higher processing temperatures) may result in producing gasoline blending products having a relatively higher octane rating and/or additional olefins for alkylate production.

In some embodiments, distillation conditions in the atmospheric distillation unit 12 may be controlled, for example, based at least in part on analysis of samples of the hydrocarbon feedstock 20 (e.g., a blended hydrocarbon feedstock from a combination of raw hydrocarbon feedstock sources) for properties and/or contents. The properties and/or content of the hydrocarbon feedstock 20 may be used, for example, to predict the yields of finished products from the refining operation, and/or to control aspects of the refining operation to meet target yields of finished products (e.g., a volume/mass percent yield of coke, a volume/mass percent yield of dry gas, alkylate and/or a blended gasoline product of a certain octane rating, and/or other products). The properties and/or content, such as (a) polynuclear aromatic (PNA) concentrations for the prediction of cold side properties (e.g., fuel freeze points, cloud points, and pour) and/or (b) sulfur content. Freeze points and/or cloud points may be important characteristics for winter blends of refinery products, such as, for example, jet fuel and diesel fuel. Target freeze and/or cloud points may thus be used as a control constraint, for example, where the draw rate of distillation products containing kerosine and jet fuel (e.g., distillation products 42d shown in FIGS. 1 and 2) may be adjusted to improve and/or maximize certain jet fuel and diesel products that may be relatively more useful and/or more efficient to produce.

In some embodiments, the unit materials resulting from a distillation process may include unit material properties, such as, for example, an amount of butane-free gasoline, an amount of total butane, an amount of dry gas, an amount of coke, an amount of gasoline, an octane rating, an amount of light fuel oil, an amount of heavy fuel oil, an amount of hydrogen sulfide, an amount of sulfur in light fuel oil, and/or an aniline point of light fuel oil. For example, control (e.g., prescriptive control) based at least in part on analysis of distillate products from one or more of the fractionation units (e.g., the LGO and HGO in distillation products 42d and 42e, respectively, from atmospheric distillation and the LVGO and HVGO distillation products 58a from vacuum distillation) may be used for the optimization of refinery-wide large fractions, such as the naphtha pool and/or distillate pool. The control may thereby allow for more effective utilization of downstream finished and unfinished products (e.g., the prioritization of naphtha, jet fuel, kerosine, and/or other products) depending at least in part on their usefulness and/or value in different markets relative to other product streams.

For example, controlling of the operation of the strippers (e.g., 206b through 206e shown in FIG. 2) positioned to remove products from the atmospheric distillation unit 12 may involve controlling one or more of the unit materials in the distillation streams (e.g., 202b through 202e) into a stripper to optimize flash. In some embodiments, at least some of the unit materials may include naphtha. In at least some such embodiments, the predicted sample properties or contents of the distillate streams 202b through 202e and/or distillation products 42a through 42e may be used as control constraints in the respective strippers 206b through 206e on the light end (T5, T10) and/or on the heavy end (T90, T95, T98 or FBP). In some embodiments, the low end components and/or heavy end components may be used to improve optimize the distillate pool for managing the production of, for example, kerosine and/or jet fuel. In some embodiments, the boiling points and/or properties associated with heavy end components (e.g., T90, T95, T98 or FBP) may be used as a control constraint when increasing or maximizing naphtha production over the production of kerosine and/or jet fuel. Alternatively, or in addition, the boiling points, flash points, and/or other properties of the distillate streams 202b through 202e and/or (after stripping) the distillation products 42a through 42e may be used as control constraints when increasing or maximizing the production of, for example, diesel over kerosine, or diesel over gas oil (LGO and HGO).

In some embodiments, the atmospheric distillation unit 12 may also include one or more high-pressure separators. At least some of the unit materials (e.g., distillation products 42a through 42e) produced by the atmospheric distillation unit 12 may include one or more of high-pressure separator water content or stripper bottoms water content. The one or more process controller(s) 306 associated with the distillation unit control assembly 300 may be configured to control, based at least in part on the one or more of the high-pressure separator water content or the stripper bottoms water content, a temperature in the high-pressure separator.

In some embodiments, the predicted flash points of distillation products containing kerosine and jet fuel (e.g., distillation products 42d) determined from the properties or contents obtained from analysis by the one or more spectroscopic analyzers may be used as control constraints when increasing or maximizing the production of kerosine and jet fuel over the production of naphtha. For example, increasing the stripping steam rate on a stripper of distillation products containing kerosine (or alternately, additional reboiler duty of the same stripper) may result in removal of more light ends from the corresponding distillate stream 202d, and may thereby increase the flash point of the kerosine product. Increasing the flash point of the kerosine product may therefore increase kerosine production by drawing less naphtha products higher up the column of the atmospheric distillation unit 12.

In some embodiments, additional material properties predicted from the feedstocks and products of a fractionation and/or distillation process may include, for example, one or more of a Research Octane Number (RON), a Motor Octane Number (MON), Anti-Knock Index (AKI), a total aromatics content, a total asphaltene content, a total butane content, a total olefins content, an amount of gasoline, an amount of propane, an amount of pentane, an aniline point, an amount of benzene, an amount of toluene, an amount of xylene, total Benzene Toluene Xylene (BTX) content, total Benzene Toluene Ethylbenzene Xylene (BTEX) content, a vapor pressure, a drivability index, a density, a specific gravity, an American Petroleum Institute (API) gravity, an amount of alcohol, an Ethanol (EtOH) kick prediction, amount of dry gas, an amount of coke, a gasoline octane, an amount of LGO, an amount of HGO, an amount of sulfur, an amount of hydrogen sulfide (H₂S), an amount of crude light ends (C5s), an amount of raw crude water, an amount of desalted crude water, LGO micro carbon residue testing (MCRT), HGO MCRT, an LGO flash, an LGO T90 temperature, an LGO T98, a kerosine flash, a kerosine T10, a kerosine T98, and/or a naphtha T98.

Assemblies, systems, apparatuses, and processes for enhanced control of the processing of heavy hydrocarbon products (e.g., in FIG. 1, atmospheric distillation bottoms in distillation products 42f, bottoms and residuum in distillation products 58c from the vacuum distillation process, and/or other residual products) are also disclosed. These heavy products may cause complications within other areas of the refining process including, for example, heat exchanger fouling, coking on the surfaces in downstream processing units, and/or an undesirably high sulfur content in downstream products, such as gasoline and diesel fuels. Determining the properties and/or contents of heavy hydrocarbon products by direct, on-line measurement, for example, using one or more spectroscopic analyzers, as described herein, may improve or optimize the production of desired products and decrease or minimize complications within the refining operation.

Referring to FIG. 1, in some embodiments, heat added by the vacuum unit heater 54 may be controlled for incoming distillation products 42f in the feedstock 20 for the vacuum distillation unit 14, for example, due at least in part to the relative ease of cracking of paraffins in the feedstock (e.g., as compared, for example, to naphthenes). Coke precursors, such as asphaltenes, may tend to break down into coke at higher temperatures. In some embodiments, one or more spectroscopic analyzer(s) 304 may be used with one or more process controller(s) 306 to enhance the operation of the vacuum distillation unit 14 to reduce one or more of these possible issues. Control of the properties and/or content and feedstock parameters (e.g., temperature) for the feedstock for vacuum distillation (e.g., vacuum distillation products 42f in FIG. 1) and/or the conditions in the vacuum column 56 may improve the efficiency of the vacuum distillation process and operation of further processing units (e.g., second processing units 316 positioned downstream, such as a deasphalter unit 150, a coker unit, a visbreaker unit, and/or other processing units), which may receive products from the vacuum distillation unit 14. For example, boiling points determined for the distillation products 42f in the feedstock for the vacuum distillation unit 14 may be used to control the temperature of the distillation products 42f and/or the temperature in the vacuum distillation column 56, thereby to reduce the risk of coking in the vacuum distillation column 56.

In some embodiments, analysis by the one or more spectroscopic analyzer(s) 304 may provide the ability to predict sample properties, including, for example, the Watson Characterization Factor (Kw), which, in turn, may be used to estimate the upper temperature limit for the vacuum distillation process. For example, in some embodiments, an empirical correlation between Kw and the temperature may be used to estimate a target or limit temperature above which significant thermal decomposition may occur. In some embodiments, the one or more process models 320 may include a correlation between Kw and temperature. In some embodiments, a spectroscopic analysis of distillation products 42f (FIGS. 1 and 2) may be used to identify a high Kw (e.g., which may be indicative of relatively greater paraffinic content), and the one or more process controller(s) 306 may be configured to communicate one or more processing units control signals(s) 318 configured to reduce the temperature in the vacuum distillation column 56, for example, to reduce the paraffinic content.

The production of coke may also be affected by inhibitors that may be predicted for portions of the vacuum distillation process and downstream processes by analyzing, for example, Conradson carbon residue (also known as “Concarbon” or “CCR”) content in the incoming distillation products 42f in the feedstock and/or by analyzing heavy vacuum gas oil products, such as distillation products 42e. Inhibitors may be present naturally or they may be added via processing. A correlation may exist between a boiling range of a hydrocarbon material and the concentration of inhibitors present. The effects of inhibitors may be temporary or lasting, depending on, for example, the type of inhibitor present. For example, Nitrogen inhibitors may provide temporary effects, while heavy metals (e.g., nickel, vanadium, iron, and/or copper) may have a more lasting effect through being quantitatively transferred from a hydrocarbon material or feedstock to a catalyst and/or other processing materials. “Metals poisoning” may result in relatively higher dry gas yields, hydrogen factors, coke yields as a percent of conversion, and/or relatively lower gasoline yields.

Referring to FIGS. 1 and 3, in some embodiments, the one or more process controller(s) 306 may be configured to determine the products and/or contents of vacuum distillation products 58a through 58c from the operation of the vacuum distillation unit 14. Vacuum distillation products 58a through 58c may provide feedstocks for the one or more second processing units 316, which may each have particular feedstock requirements and/or process parameters. The one or more spectroscopic analyzers 62a through 62c may be configured to predict properties and/or contents of the respective vacuum distillation products 58a through 58c, and one or more signals indicative of the properties and/or contents may be communicated to the one or more process controller(s) 306. In some embodiments, the process model 320 may, for example, include a band of temperatures (a decomposition band) below which coking risk is negligible in the vacuum distillation process, and an area within the band of temperatures that represents uncertainty and/or the probability of coking in the vacuum distillation process. The distillation unit control assembly 300 (FIG. 3) may be configured to utilize the process model 320 and data from the one or more spectroscopic analyzers 304 to substantially maintain temperature in the vacuum distillation column 56 at or below a lower temperature of the band of temperatures.

In some embodiments of refining operation 10, vacuum distillation products 58a and 58b may, for example, be blended into residual fuels. In some embodiments, when vacuum distillate in vacuum distillation products 58a and 58b is to be maximized, the amount of gas oil allowed to remain in the vacuum residue or bottoms in vacuum distillation products 58c may be minimized. The process controller(s) 306 may be configured to control the volume fraction and/or percentages of the vacuum distillation products 58c yielded as vacuum residues or bottoms, for example, based at least in part on one or more properties predicted by, for example, the one or more spectroscopic analyzers 62c (FIG. 1).

In some embodiments, for example as shown in FIG. 1, vacuum distillation products 58a, such as gas oils, may be supplied as a feedstock for catalytic cracking in the FCC unit 44. In some embodiments, one or more properties, such as carbon content, may be determined in stream 58a by one or more spectroscopic analyzers 62a, and one or more signals indicative of the one or more properties may be communicated to the process controller(s) 306. In some embodiments, it may be desirable for feedstocks for the FCC unit 44 to contain minimal carbon content, which may result in relatively reduced coke formation on the cracking catalyst used in the FCC unit 44. Other second downstream processing units, such as, for example, hydrotreaters and/or hydrocrackers, may be supplied with feedstocks containing a relatively high metals content, but it may be desirable to supply them with feedstocks having relatively minimal carbon content and/or asphalt content, for example, to minimize coke formation. In some embodiments, distillate fuel oils and/or other products present in the vacuum distillation products 58b may be used directly as finished products. Properties and/or content, such as API gravity, viscosity metal content, flash point, and/or other properties specified for these finished products may be analyzed and determined, for example, by one or more spectroscopic analyzers 62b. The one or more process controller(s) 306 may thereby be configured to adjust temperatures and/or other parameters in the atmospheric distillation processes and/or the vacuum distillation process, such that the properties of the finished products measured in vacuum distillation products 58b responsively converge on one or more of target properties.

In some embodiments, one or more spectroscopic analyzer(s) 304 and process controllers 306 may be configured to enhance control of other fractionation units, such as, for example, the saturated gas unit 16 (FIG. 1). For example, one or more spectroscopic analyzer(s) (e.g., spectroscopic analyzer 46a) may be configured to predict properties and/or contents of a feedstock provided to the saturated gas unit 16 (e.g., the overhead products, such as distillation products 42a from the atmospheric distillation unit 12). One or more signals generated by the one or more spectroscopic analyzer(s) 304 may be communicated to the one or more process controllers 306 (FIG. 3), which may be configured to control and/or maintain stability of the distillation products 42a, for example, so that the saturated gas unit 16 may produce substantially consistent fractions. Based at least in part on the one or more signals received from the spectroscopic analyzers, the one or more process controllers 306 may communicate one or more control signals, for example, to balance a fuel header in a fuel system of the saturated gas unit 16, minimize the purchase of off-plot natural gas for the saturated gas unit 16, maintain a minimum heating value for the saturated gas unit 16 by vaporizing propane, and/or specifying C3 targets or other targets for components (e.g., absorbers, strippers, condensers, etc.) of the saturated gas unit 16. In some embodiments, the feedstocks supplied to one or more second downstream processing units 316 associated with operation of the saturated gas unit 16 (e.g., processing units for isomerization, polymerization, Merox treatment, and/or alkylation) may be improved or optimized, for example, via enhanced control of the saturated gas unit 16.

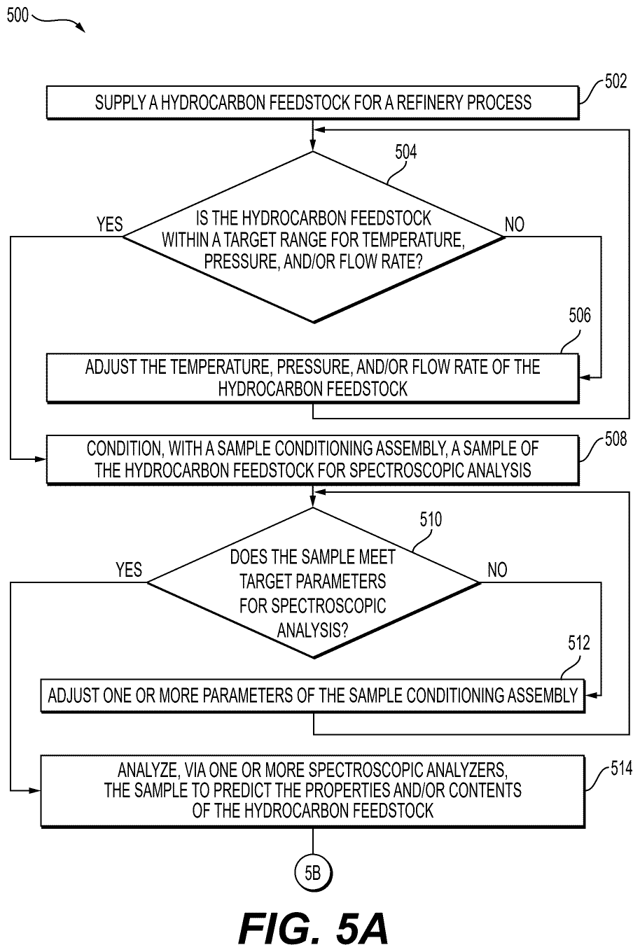

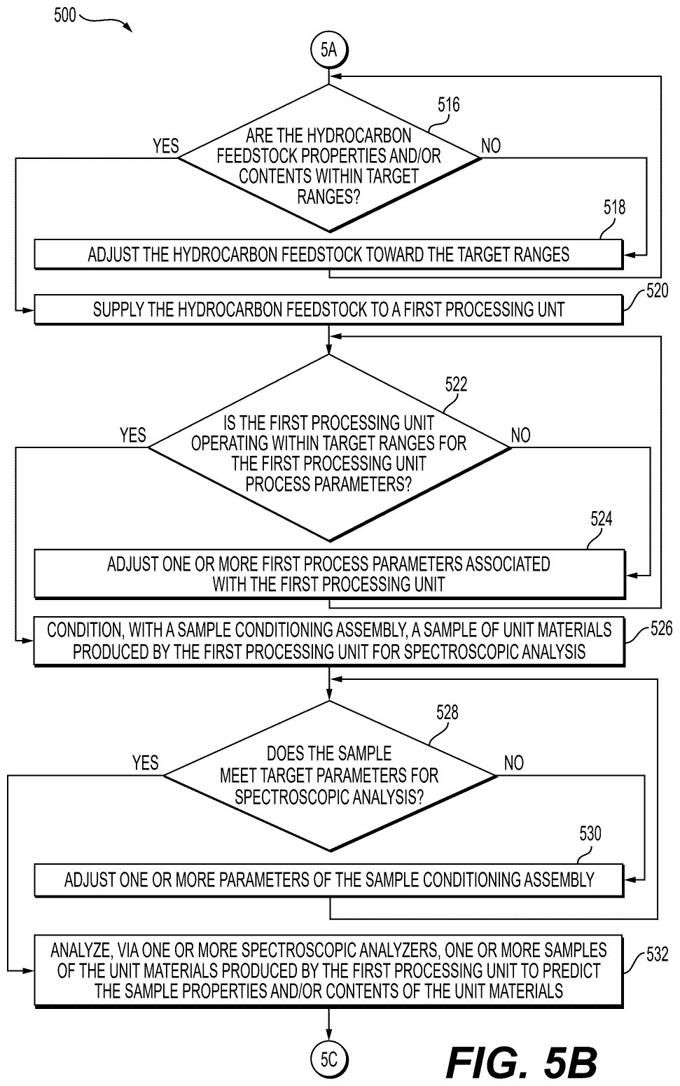

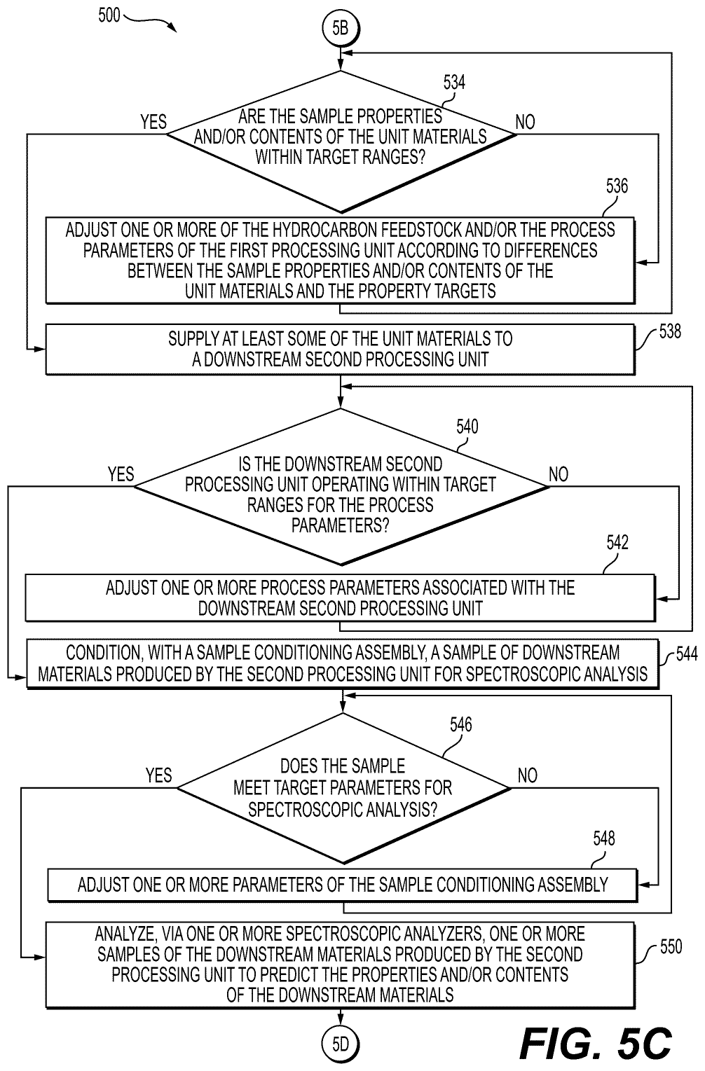

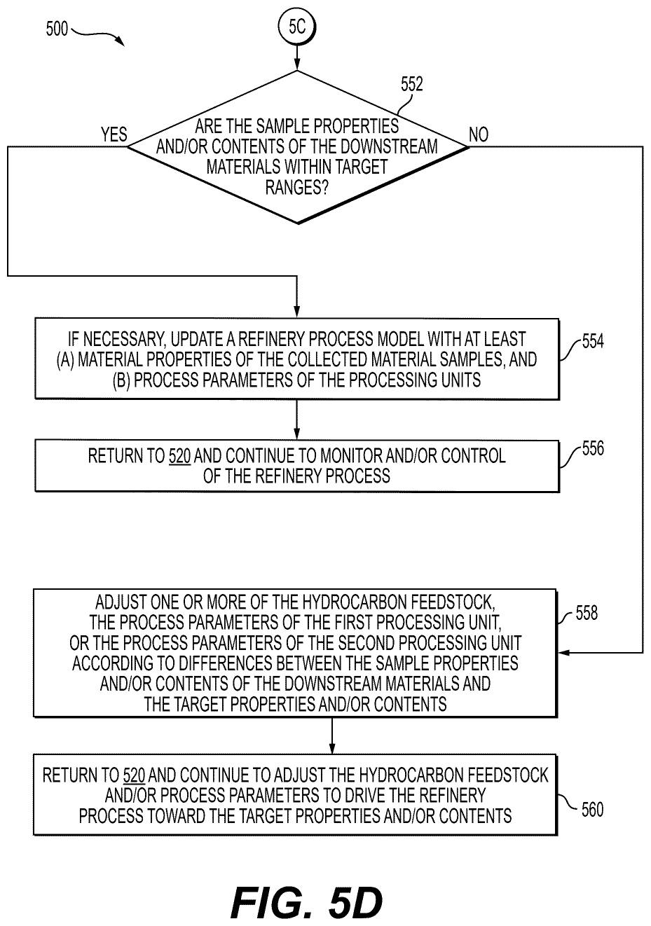

FIG. 5A, FIG. 5B, FIG. 5C, and FIG. 5D are a block diagram of an example method 500 for enhancing control of a refinery process associated with a petroleum refining operation, according to embodiments of the disclosure. The example method 500 is illustrated as a collection of blocks in a logical flow graph, which represent a sequence of operations. In the context of software, where applicable, the blocks represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described blocks may be combined in any order and/or in parallel to implement the method.

Referring to FIG. 5A, at 502 the example method 500 may include supplying hydrocarbon feedstock for a refinery process, for example, as described herein. At 504, the example method 500 may include determining whether the hydrocarbon feedstock is within a target temperature range, a target pressure range, and/or a target flow rate, for example, as described herein.

If, at 504, it is determined that the temperature, pressure, or flow rate is not within a target temperature range, a target pressure range, and/or a target flow rate, at 506, the example method 500 may include adjusting the temperature, the pressure, and/or the flow rate of the hydrocarbon feedstock to be within the target ranges, and returning to 504 to repeat the determination. Alternatively, or in addition, one or more correction factors (e.g., a correction factor based on past operational data) may be applied to the temperature, pressure, and/or flow rate, for example, where adjustment is difficult or not possible within the refining process.

If, at 504, it is determined that the temperature, pressure, and/or target flow rate are within the target ranges, at 508, the example method 500 may include conditioning, via a sample conditioning assembly, a sample of the hydrocarbon feedstock for analysis by one or more spectroscopic analyzers, for example, as described herein.

At 510, the example method 500 may include determining whether the conditioned hydrocarbon feedstock sample is within target parameters for analysis by the one or more spectroscopic analyzers. This may include determining whether water, particulates, and/or other contaminates have been removed from the conditioned hydrocarbon feedstock sample, and/or whether the conditioned sample is within a desired predetermined temperature range for improving the accuracy of the analysis by the spectroscopic analyzer(s).

If, at 510, it is determined that the conditioned hydrocarbon feedstock sample is not within target parameters for analysis, the example method 500, at 512, may include adjusting one or more parameters associated with operation of the sample conditioning assembly, such that the conditioned hydrocarbon feedstock sample is within the target parameters, and returning to 510 to repeat the determination.

If, at 510, it is determined that the conditioned hydrocarbon feedstock sample is within target parameters for analysis, the example method 500, at 514, may include supplying the conditioned hydrocarbon feedstock sample to the spectroscopic analyzer(s) and analyzing, via the spectroscopic analyzer(s), the conditioned hydrocarbon feedstock sample to predict (or determine) hydrocarbon feedstock properties and/or contents, for example, as described herein.

Referring to FIG. 5B, at 516, the example method 500 may include determining whether the hydrocarbon feedstock properties and/or contents are within desired ranges of target properties and/or target contents for the hydrocarbon feedstock.

If, at 516, it is determined that the hydrocarbon feedstock properties are not within the desired ranges, the example method 500, at 518, may include adjusting the hydrocarbon feedstock toward the target properties and/or target contents to be within the desired ranges of target properties and/or target contents for the hydrocarbon feedstock, and returning to 516 to repeat the determination.

If, at 516, it is determined that the hydrocarbon feedstock properties are within the desired ranges of the target properties and/or target contents for the hydrocarbon feedstock, the example method 500, at 520, may include supplying the hydrocarbon feedstock to a first processing unit (e.g., a fractionation unit, such as an atmospheric distillation unit, a vacuum distillation unit, and/or a saturated gas unit), for example, as described herein.

At 522, the example method 500 may include determining whether the first processing unit is operating within desired target ranges for the first processing unit process parameters. The determining may include, for example, comparing the current process parameters of the first processing unit with one or more sets of predetermined operating conditions or target process parameters for the first processing unit.

If, at 522, it is determined that the first processing unit is not operating within desired target ranges for the first processing unit process parameters, the example method 500, at 524, may include adjusting one or more first processing unit process parameters associated with the first processing unit, and returning to 522 to repeat the determination.

If, at 522, it is determined that the first processing unit is operating within desired target ranges for the process parameters of the first processing unit, the example method 500, at 526, may include conditioning, via the sample conditioning assembly, a sample of unit materials produced by the first processing unit for analysis by one or more spectroscopic analyzers, for example, as described herein.

At 528, the example method 500 may include determining whether the conditioned unit material sample is within target parameters for analysis by the spectroscopic analyzer. This may include determining whether water, particulates, and/or other contaminates have been removed from the conditioned unit material sample, and/or whether the conditioned sample is within a desired predetermined temperature range for improving the accuracy of the analysis by the spectroscopic analyzer(s).

If, at 528, it is determined that the conditioned unit material sample is not within target parameters for analysis, the example method 500, at 530, may include adjusting one or more parameters associated with operation of the sample conditioning assembly, such that the conditioned unit material sample is within the target parameters, and returning to 528 to repeat the determination.

If, at 528, it is determined that the conditioned unit material sample is within target parameters for analysis, the example method 500, at 532, may include supplying the conditioned unit material sample to the spectroscopic analyzer(s) and analyzing, via the spectroscopic analyzer(s), the conditioned unit material sample to predict (or determine) unit material properties and/or contents, for example, as described herein.

At 534 (FIG. 5C), the example method 500 may include determining whether the sample properties of the unit materials are within target ranges.

If, at 534, it is determined that the sample properties of the unit materials are not within target ranges, the example method 500, at 536, may include adjusting one or more of the hydrocarbon feedstock or the process parameters of the first processing unit according to differences between the sample properties of the unit materials and the target properties, and returning to 534 to repeat the determination.

If, at 534, it is determined that the sample properties of the first intermediate materials are within target ranges, the example method 500, at 538, may include supplying at least some of the unit materials to a downstream second processing unit, for example, as described herein.

At 540, the example method 500 may include determining whether the downstream second processing unit is operating within target ranges for the process parameters of the downstream second processing unit.

If, at 540, it is determined that the downstream second processing unit is not operating within target ranges for the process parameters of the downstream second processing unit, the example method 500, at 542, may include adjusting one or more process parameters associated with operation of the downstream second processing unit, and returning to 540 to repeat the determination.

If, at 540, it is determined that the downstream second processing unit is operating within target ranges for the process parameters of the downstream second processing unit, the example method 500, at 544, the example method may include conditioning, via a sample conditioning assembly, a sample of downstream materials produced by the second processing unit for analysis by one or more spectroscopic analyzers, for example, as described herein.

At 546, the example method 500 may include determining whether the conditioned downstream material sample is within target parameters for analysis by the spectroscopic analyzer(s). This may include determining whether water, particulates, and/or other contaminates have been removed from the conditioned downstream material sample, and/or whether the conditioned sample is within a desired predetermined temperature range for improving the accuracy of the analysis by the spectroscopic analyzer(s).

If, at 546, it is determined that the conditioned downstream material sample is not within target parameters for analysis, the example method 500, at 548, may include adjusting one or more parameters associated with operation of the sample conditioning assembly, such that the conditioned downstream material sample is within the target parameters, and returning to 546 to repeat the determination.

If, at 546, it is determined that the conditioned downstream material sample is within target parameters for analysis, the example method 500, at 550, may include supplying the conditioned downstream material sample to the spectroscopic analyzer(s) and analyzing, via the spectroscopic analyzer(s), the conditioned downstream material sample to predict (or determine) downstream material properties and/or contents, for example, as described herein.

At 552 (FIG. 5D), the example method 500 may include determining whether the sample properties and/or contents of the downstream materials are within target ranges of the properties and/or contents.

If, at 552, it is determined that the sample properties of the downstream materials are within target ranges, the example method 500, at 554 may include updating a refinery process model with at least (a) material properties of the collected material samples and (b) operating parameters of the processing units, for example, as described herein. The refinery process model may include, for example, a fractional distillation model, and/or a model for one or more other refining processes occurring on-line during operations of the refinery process. the material properties of the collected material samples and/or operating parameters of the processing units may be included as at least a portion of fractional distillation process data that may be used, for example, to update the one or more process models.

At 556, the example method 500 may include returning to 520 and continuing to monitor and/or control the refinery process.

If, at 552, it is determined that the sample properties of the downstream materials are not within target property and/or target content ranges, the example method 500, at 558, may include adjusting one or more of the hydrocarbon feedstock, the process parameters of the first processing unit, or the process parameters of the downstream second processing unit, according to differences between the sample properties of the downstream materials and the property targets and/or content targets. The example method 500, at 560, may include returning to 520 and continuing to adjust the hydrocarbon feedstock and/or process parameters to drive the refinery process towards the target properties and/or target contents.

In some embodiments, the example method 500 may result in causing the refinery process to produce one or more of: (a) intermediate materials having one or more properties within a selected range of one or more target properties and/or target contents of the intermediate materials, (b) unit materials having one or more properties within a selected range of one or more target properties and/or target contents of the unit materials, or downstream materials having one or more properties within a selected range of one or more target properties and/or target contents of the downstream materials. In some embodiments, this may cause the refinery process to achieve material outputs that more accurately and responsively converge on one or more of the target properties and/or target contents. In some embodiments, the example method may result in optimizing one or more of: (a) one or more target properties and/or target contents of the one or more intermediate materials, (b) one or more target properties and/or target contents of the one or more unit materials, (c) or one or more target properties and/or target contents of one or more downstream materials produced by the one or more downstream second processing units, thereby to optimize the refinery process to achieve material outputs that more accurately and responsively converge on one or more of the target properties and/or target contents.

Those skilled in the art will also appreciate that aspects of the subject matter described herein may be practiced on or in conjunction with other computer system configurations beyond those described herein, including multiprocessor systems, microprocessor-based or programmable consumer electronics, minicomputers, mainframe computers, handheld computers, mobile telephone devices, tablet computing devices, special-purposed hardware devices, network appliances, and the like. Some or all of the computer system configurations may be connected to a network.

References are made to block diagrams of systems, methods, apparatuses, and computer program products according to example embodiments. It will be understood that at least some of the blocks of the block diagrams, and combinations of blocks in the block diagrams, may be implemented at least partially by computer program instructions. These computer program instructions may be loaded onto a general purpose computer, special purpose computer, special purpose hardware-based computer, or other programmable data processing apparatus to produce a machine, such that the instructions which execute on the computer or other programmable data processing apparatus implement the functionality of at least some of the blocks of the block diagrams, or combinations of blocks in the block diagrams discussed.

These computer program instructions may also be stored in a non-transitory computer-readable memory that can direct a computer or other programmable data processing apparatus to function in a particular manner, such that the instructions stored in the computer-readable memory produce an article of manufacture including instruction means that implement the function specified in the block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide task, acts, actions, or operations for implementing the functions specified in the block or blocks.

One or more components of the systems and one or more elements of the methods described herein may be implemented through an application program running on an operating system of a computer. They may also be practiced with other computer system configurations, including hand-held devices, multiprocessor systems, microprocessor-based or programmable consumer electronics, mini-computers, mainframe computers, and the like. While the subject matter described herein is presented in the general context of program modules that execute on one or more computing devices, those skilled in the art will recognize that other implementations may be performed in combination with other types of program modules. Generally, program modules may include routines, programs, components, data structures, and other types of structures that perform particular tasks or implement particular abstract data types.

Application programs that are components of the systems and methods described herein may include routines, programs, components, data structures, etc. that may implement certain abstract data types and perform certain tasks or actions. In a distributed computing environment, the application program (in whole or in part) may be located in local memory or in other storage. In addition, or alternatively, the application program (in whole or in part) may be located in remote memory or in storage to allow for circumstances where tasks can be performed by remote processing devices linked through a communications network. Those skilled in the art will also appreciate that aspects of the subject matter described herein may be practiced on or in conjunction with other computer system configurations beyond those described herein, including multiprocessor systems, microprocessor-based or programmable consumer electronics, minicomputers, mainframe computers, handheld computers, mobile telephone devices, tablet computing devices, special-purposed hardware devices, network appliances, and the like.

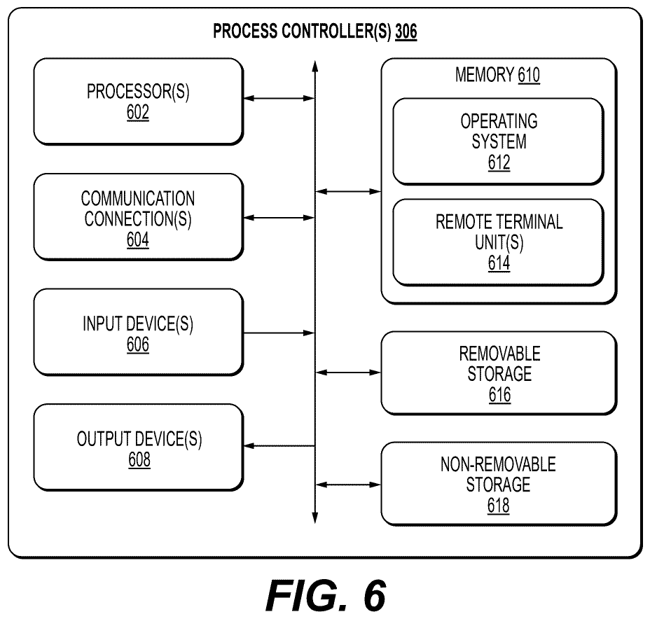

FIG. 6 is a schematic diagram of example process controller(s) 306 configured to at least partially control a one or more fractionation units and/or refinery processes, according to embodiments of the disclosure, for example, as described herein. The process controller(s) 306 may include one or more processor(s) 602 configured to execute certain operational aspects associated with implementing certain systems and methods described herein. The processor(s) 602 may communicate with a memory 610. The processor(s) 602 may be implemented and operated using appropriate hardware, software, firmware, or combinations thereof. Software or firmware implementations may include computer-executable or machine-executable instructions written in any suitable programming language to perform the various functions described. In some examples, instructions associated with a function block language may be stored in the memory 610 and executed by the processor(s) 602.

The memory 610 may be used to store program instructions that are loadable and executable by the processor(s) 602, as well as to store data generated during the execution of these programs. Depending on the configuration and type of the process controller(s) 306, the memory 610 may be volatile (such as random access memory (RAM)) and/or non-volatile (such as read-only memory (ROM), flash memory, etc.). In some examples, the memory devices may include additional removable storage 616 and/or non-removable storage 618 including, but not limited to, magnetic storage, optical disks, and/or tape storage. The disk drives and their associated computer-readable media may provide non-volatile storage of computer-readable instructions, data structures, program modules, and other data for the devices. In some implementations, the memory 610 may include multiple different types of memory, such as static random access memory (SRAM), dynamic random access memory (DRAM), or ROM.

The memory 610, the removable storage 616, and the non-removable storage 618 are all examples of computer-readable storage media. For example, computer-readable storage media may include volatile and non-volatile, removable and non-removable media implemented in any method or technology for storage of information such as computer-readable instructions, data structures, program modules or other data. Additional types of computer storage media that may be present may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile discs (DVD) or other optical storage, magnetic cassettes, magnetic tapes, magnetic disk storage or other magnetic storage devices, or any other medium which may be used to store the desired information and which may be accessed by the devices. Combinations of any of the above should also be included within the scope of computer-readable media.

The process controller(s) 306 may also include one or more communication connection(s) 604 that may facilitate a control device (not shown) to communicate with devices or equipment capable of communicating with the process controller(s) 306. The process controller(s) 306 may also include a computer system (not shown). Connections may also be established via various data communication channels or ports, such as USB or COM ports to receive cables connecting the process controller(s) 306 to various other devices on a network. In some examples, the process controller(s) 306 may include Ethernet drivers that enable the process controller(s) 306 to communicate with other devices on the network. According to various examples, communication connections 604 may be established via a wired and/or wireless connection on the network.

The process controller(s) 306 may also include one or more input devices 606, such as a keyboard, mouse, pen, voice input device, gesture input device, and/or touch input device. It may further include one or more output devices 608, such as a display, printer, and/or speakers. In some examples, computer-readable communication media may include computer-readable instructions, program modules, or other data transmitted within a data signal, such as a carrier wave or other transmission. As used herein, however, computer-readable storage media may not include computer-readable communication media.

Turning to the contents of the memory 610, the memory 610 may include, but is not limited to, an operating system (OS) 612 and one or more application programs or services for implementing the features and embodiments disclosed herein. Such applications or services may include remote terminal units 614 for executing certain systems and methods for controlling operation of the processing units and/or refinery processes (e.g., semi- or fully-autonomously controlling operation of the processing units and/or refinery processes), for example, upon receipt of one or more control signals generated by the process controller(s) 306. In some embodiments, one or more remote terminal unit(s) 614 may be located in the vicinity of one or more of the processing units. The remote terminal unit(s) 614 may reside in the memory 610 or may be independent of the process controller(s) 306. In some examples, the remote terminal unit(s) 614 may be implemented by software that may be provided in configurable control block language and may be stored in non-volatile memory. When executed by the processor(s) 602, the remote terminal unit(s) 614 may implement the various functionalities and features associated with the process controller(s) 306 described herein.

The process controller(s) 306 of FIG. 6 is/are provided by way of example only. As desired, embodiments of the disclosure may include process controller(s) 306 with more or fewer components than are illustrated in FIG. 6. Additionally, certain components of the example process controller(s) 306 shown in FIG. 6 may be combined in various embodiments of the disclosure.

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Those skilled in the art should appreciate that the parameters and configurations described herein are exemplary and that actual parameters and/or configurations will depend on the specific application in which the systems and techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto, the disclosure may be practiced other than as specifically described.

The present application describes and discloses systems and methods for enhancing process controls according to any of the following examples:

Clause 1. A method for enhancing control of a refining process associated with a petroleum refining operation, the method comprising: conditioning a hydrocarbon feedstock sample of a hydrocarbon feedstock supplied to one or more first processing units associated with the petroleum refining operation, the conditioning including one or more of: (a) filtering the hydrocarbon feedstock sample, (b) changing a temperature of the hydrocarbon feedstock sample, (c) diluting the hydrocarbon feedstock sample in solvent, or (d) degassing the hydrocarbon feedstock sample, the hydrocarbon feedstock having one or more hydrocarbon feedstock properties and the one or more first processing units including one or more fractionation units; analyzing the hydrocarbon feedstock sample via a first spectroscopic analyzer to provide hydrocarbon feedstock sample spectra; predicting one or more hydrocarbon feedstock sample properties associated with the hydrocarbon feedstock sample based at least in part on the hydrocarbon feedstock sample spectra; operating the one or more first processing units to produce one or more unit materials, the one or more unit materials having one or more unit materials properties, and the one or more unit materials comprising one or more of intermediate materials or unit product materials; conditioning a unit material sample from the one or more unit materials to one or more of: (a) filter the unit material sample, (b) change a temperature of the unit material sample, (c) dilute the unit material sample in solvent, or (d) degas the unit material sample; analyzing the unit material sample via one or more of the first spectroscopic analyzer or a second spectroscopic analyzer to provide unit material sample spectra, the one or more of the first spectroscopic analyzer or the second spectroscopic analyzer being calibrated to generate standardized spectral responses; predicting one or more unit material sample properties associated with the unit material sample based at least in part on the unit material sample spectra; and prescriptively controlling, during the refining process, via one or more controllers based at least in part on the one or more hydrocarbon feedstock sample properties and the one or more unit material sample properties, one or more of: (a) one or more hydrocarbon feedstock properties associated with the hydrocarbon feedstock supplied to the one or more first processing units; (b) one or more intermediate properties associated with the intermediate materials produced by the one or more first processing units; (c) one or more unit product materials properties associated with the unit product materials; (d) operation of the one or more first processing units; or (e) operation of one or more second processing units positioned downstream relative to the one or more first processing units, so that the prescriptively controlling, during the refining process, causes the refining process to produce one or more of: (i) one or more intermediate materials each having one or more properties within a range of one or more target properties of the one or more intermediate materials, (ii) one or more unit product materials each having one or more properties within a range of one or more target properties of the one or more unit product materials, or (iii) one or more downstream materials each having one or more properties within a range of one or more target properties of the one or more downstream materials, thereby to cause the refining process to achieve material outputs that more accurately and responsively converge on one or more of the target properties.

Clause 2. The method of clause 1, wherein: the hydrocarbon feedstock comprises a blended hydrocarbon feedstock including a plurality of hydrocarbon feedstocks from respective hydrocarbon feedstock flows; and the prescriptively controlling the one or more hydrocarbon feedstock properties associated with the hydrocarbon feedstock supplied to the one or more first processing units comprises controlling one or more feed ratios of the respective hydrocarbon feedstock flows.

Clause 3. The method of clause 1, wherein the prescriptively controlling of one or more of (a) the one or more intermediate properties associated with the intermediate materials, or (b) the one or more unit product materials properties associated with the unit product materials, comprises: controlling one or more of: (i) content of the hydrocarbon feedstock, or (ii) operation of the one or more first processing units.

Clause 4. The method of clause 1, wherein the one or more fractionation units comprise one or more of an atmospheric distillation unit, a vacuum distillation unit, a fracking oil fractionation unit, a saturated gas unit, or a condensate fractionation unit.

Clause 5. The method of clause 1, wherein one or more of: (a) the predicting of the one or more hydrocarbon feedstock properties comprises predicting a boiling point associated with the hydrocarbon feedstock sample, and the method comprises controlling, based at least in part on the boiling point associated with the hydrocarbon feedstock sample, operation of one or more of the one or more first processing units or the one or more second processing units; or (b) the predicting of the one or more unit material sample properties comprises predicting a boiling point associated with the unit material sample, and the method comprises controlling, based at least in part on the boiling point associated with the unit material sample, operation of one or more of the one or more first processing units or the one or more second processing units.

Clause 6. The method of clause 1, wherein one or more of the first spectroscopic analyzer or the second spectroscopic analyzer comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer.

Clause 7. The method of clause 1, wherein one or more of: (a) the analyzing the hydrocarbon feedstock sample is performed on-line and in real-time; (b) the analyzing the hydrocarbon feedstock sample is performed off-line in a laboratory setting; (c) the analyzing the unit material sample is performed on-line and in real-time; or (d) the analyzing the unit material sample is performed off-line in a laboratory setting.

Clause 8. The method of clause 1, further comprising prescriptively controlling content of the one or more downstream materials via control of one or more of: (a) content of the hydrocarbon feedstock supplied to the one or more first processing units; (b) the operation of the one or more first processing units; (c) content of the intermediate materials produced by the one or more first processing units; (d) content of the unit product materials; or (e) the operation of the one or more second processing units.

Clause 9. The method of clause 1, wherein the prescriptively controlling comprises operating a prescriptive analytical model configured to improve an accuracy of one or more of: (a) the one or more hydrocarbon feedstock sample properties associated with the hydrocarbon feedstock sample; (b) the one or more unit material sample properties associated with the unit material sample; (c) content of the hydrocarbon feedstock supplied to the one or more first processing units; (d) content of the one or more intermediate materials produced by the one or more first processing units; (e) content of the unit product materials produced by the one or more first processing units; or (f) content of the downstream materials produced by the one or more second processing units.

Clause 10. The method of clause 1, further comprising one or more of: (a) prescriptively controlling one or more of the temperature of the hydrocarbon feedstock sample or the temperature of the unit material sample, thereby to substantially maintain the one or more of the temperature of the hydrocarbon feedstock sample or the temperature of the unit material sample within a respective temperature range; or (b) applying one or more temperature correction factors to one or more of: (i) the hydrocarbon feedstock sample spectra; (ii) the one or more hydrocarbon feedstock sample properties; (iii) the unit material sample spectra; or (iv) the one or more unit material properties.

Clause 11. The method of clause 1, wherein the prescriptively controlling comprises controlling one or more process parameters, the one or more process parameters comprising one or more of: (a) a flow rate of the hydrocarbon feedstock supplied to the one or more first processing units; (b) one or more feed ratios of a plurality of hydrocarbon feedstock flows combined into the hydrocarbon feedstock; (c) a pressure of the hydrocarbon feedstock supplied to the one or more first processing units; or (d) a preheating temperature of the hydrocarbon feedstock supplied to the one or more first processing units.

Clause 12. The method of clause 1, wherein one or more of: (a) the predicting of the one or more hydrocarbon feedstock sample properties comprises predicting a boiling point associated with the hydrocarbon feedstock; or (b) the predicting of the one or more unit material sample properties comprises predicting a boiling point associated with the one or more unit materials.

Clause 13. The method of clause 12, further comprising controlling, based at least in part on one or more of: (a) the boiling point associated with the hydrocarbon feedstock or (b) the boiling point associated with the one or more unit materials, operation of the one or more fractionation units.

Clause 14. The method of clause 12, further comprising predicting, based at least in part on one or more of: (a) the boiling point associated with the hydrocarbon feedstock or (b) the boiling point associated with the one or more unit materials, a yield fraction associated with the one or more unit materials.

Clause 15. The method of clause 1, wherein the unit material sample comprises naphtha, and the method further comprises controlling, based at least in part on a boiling point of the naphtha, one or more of: (a) downstream flows of one or more of intermediate materials associated with the naphtha or unit product materials associated with the naphtha; (b) downstream flows of one or more of intermediate materials associated with distillates or unit product materials associated with the distillates; (c) a ratio of downstream flows associated with the naphtha to downstream flows associated with kerosene; or (d) operation of a naphtha stripper.

Clause 16. The method of clause 15, wherein the method further comprises controlling operation of the naphtha stripper, and the controlling of the operation of the naphtha stripper comprises adjusting a hydrocarbon stream into the naphtha stripper to improve flash.

Clause 17. The method of clause 1, wherein the predicting of the one or more hydrocarbon feedstock sample properties comprises predicting one or more properties associated with desalter crude water, and the method further comprises controlling, based at least in part on the one or more properties associated with the desalter crude water, operation of an upstream desalter unit.

Clause 18. The method of clause 1, further comprising displaying, via a display, one or more of (a) the one or more hydrocarbon feedstock sample properties, or (b) the one or more unit material sample properties.

Clause 19. The method of clause 1, wherein the prescriptively controlling operation of the one or more of (a) the one or more first processing units, or (b) the one or more second processing units, comprises: comparing one or more of: (a) the one or more hydrocarbon feedstock sample properties or (b) the one or more unit material sample properties, to one or more of: (i) material properties of a material database; (ii) one or more threshold values associated with operation of one or more of: (aa) the one or more first processing units, or (bb) the one or more second processing units; or (iii) target properties.

Clause 20. The method of clause 19, wherein the target properties comprise target content associated with one or more of: (a) content of the hydrocarbon feedstock supplied to the one or more first processing units; (b) content of the intermediate materials produced by the one or more first processing units; (c) content of the unit product materials produced by the one or more first processing units; or (d) content of the downstream materials produced by the one or more second processing units.

Clause 21. The method of clause 1, wherein: at least one of the one or more first processing units comprises an atmospheric distillation unit; and the one or more hydrocarbon feedstock sample properties and the one or more unit material sample properties are comprised of one or more of: (a) a quantitative composition of a hydrocarbon feedstock including a mixture of one or more feedstocks; (b) densities of products of the atmospheric distillation unit and viscosities of the products of the atmospheric distillation unit; or (c) one or more of (i) paraffinic content or (ii) aromatic content, of products of the atmospheric distillation unit.

Clause 22. The method of clause 1, wherein: at least one of the one or more first processing units comprises an atmospheric distillation unit; and the one or more unit material sample properties are comprised of one or more of: API gravity, UOP K factor, distillation points, Coker gas oil content, carbon residue content, nitrogen content, sulfur content, saturates content, thiophene content, single-ring aromatics content, dual-ring aromatics content, triple-ring aromatics content, or quad-ring aromatics content.

Clause 23. The method of clause 1, wherein the one or more hydrocarbon feedstock sample properties and the one or more unit material sample properties are comprised of a content ratio indicative of relative amounts of one or more hydrocarbon classes present in one or more of (a) the hydrocarbon feedstock sample or (b) the unit material sample.

Clause 24. The method of clause 1, wherein the one or more unit material sample properties are comprised of one or more of: an amount of butane-free gasoline, an amount of total butane, an amount of dry gas, an amount of coke, an amount of gasoline, octane rating, an amount of light fuel oil, an amount of heavy fuel oil, an amount of hydrogen sulfide, an amount of sulfur in light fuel oil, or an aniline point of light fuel oil.

Clause 25. The method of clause 1, wherein: the one or more unit material sample properties are comprised of one or more of: pentane content, raw crude water content, desalted crude water content, heavy atmospheric gas oil (HAGO) content, light atmospheric gas oil (LAGO) flash, or kerosene flash point; and the method further comprises controlling, based at least in part on the one or more of: the pentane content, the raw crude water content, the desalted crude water content, the (HAGO content, the LAGO flash, or the kerosene flash point, one or more of: crude blend, make-up water, desalter severity, HAGO wash rate, stripping, LAGO draw rate, stripping steam, or kerosene draw associated with the one or more of the first processing units.

Clause 26. The method of clause 1, wherein: the one or more unit material sample properties comprises one or more of: ethane content, propane content, propene content, isobutane content, or n-butane content; and the method further comprises controlling, based at least in part on the one or more of the ethane content, the propane content, the propene content, the isobutane content, or the n-butane content, one or more of: absorber pressure, lean oil flow rate, lean oil temperature, high-pressure separator temperature, reactor conversion, or stripper reboiler duty.

Clause 27. The method of clause 1, wherein: the one or more unit material sample properties are comprised of one or more of high-pressure separator water content or stripper bottoms water content; and the method further comprises controlling, based at least in part on the one or more of the high-pressure separator water content or the stripper bottoms water content, a temperature of a high-pressure separator.

Clause 28. The method of clause 1, wherein: (a) the one or more first processing units comprises an atmospheric distillation unit comprising an atmospheric column; (b) the method comprises: (i) supplying the hydrocarbon feedstock to the atmospheric column; (ii) separating, via the atmospheric column, the hydrocarbon feedstock into a plurality of unit materials; (iii) separating one or more intermediate samples from one or more locations of the atmospheric column; (iv) separating one or more unit material samples from the at least one of the plurality of unit materials; (v) analyzing, via one or more of the first spectroscopic analyzer, the second spectroscopic analyzer, or one or more additional spectroscopic analyzers, the one or more intermediate samples; (vi) predicting, based at least in part on the analyzing of the one or more intermediate samples, one or more intermediate sample properties associated with the one or more intermediate samples; (vii) analyzing, via one or more of the first spectroscopic analyzer, the second spectroscopic analyzer, or the one or more additional spectroscopic analyzers, the one or more unit material samples; and (viii) predicting, based at least in part in the analyzing of the one or more unit material samples, one or more unit material sample properties associated with the one or more unit material samples; and (c) the prescriptively controlling is based at least in part on one or more of (i) the one or more intermediate sample properties or (ii) the one or more unit material sample properties.

Clause 29. The method of clause 28, further comprising supplying one or more of the plurality of unit materials to a saturated gas unit associated with the atmospheric distillation unit.

Clause 30. The method of clause 28, wherein the atmospheric distillation unit further comprises a heat exchanger and a return conduit, and the method further comprises: changing, via the heat exchanger, a temperature of at least one of the plurality of unit materials, thereby to provide a temperature-controlled unit materials stream; and returning, via the return conduit, at least a portion of the temperature-controlled unit materials stream to the atmospheric column.

Clause 31. The method of clause 28, wherein the atmospheric distillation unit further comprises one or more side strippers, and the method further comprises separating, via the one or more side strippers, at least one of the plurality of unit materials from the atmospheric column.

Clause 32. The method of clause 28, further comprising one or more of: (a) prior to the analyzing the one or more intermediate samples, conditioning the one or more intermediate samples, thereby to provide one or more conditioned intermediate samples; or (b) prior to the analyzing the one or more unit material samples, conditioning the one or more unit material samples, thereby to provide one or more conditioned unit material samples.

Clause 33. The method of clause 28, wherein the prescriptively controlling comprises controlling one or more operating parameters associated with the one or more fractionation units against operating constraints of the one or more fractionation units.

Clause 34. The method of clause 28, wherein: (a) the one or more first processing units further comprises a vacuum distillation unit comprising a vacuum column positioned to receive a residue stream from the atmospheric column; (b) the method comprises: (i) separating, via the vacuum column, the residue stream into one or more intermediate process feedstocks; (ii) separating one or more intermediate process feedstock samples from the one or more intermediate process feedstocks; (iii) analyzing, via one or more of the first spectroscopic analyzer, the second spectroscopic analyzer, or the one or more additional spectroscopic analyzers, the one or more intermediate process feedstock samples; and (iv) predicting, based at least in part in the analyzing of the one or more intermediate process feedstock samples, one or more intermediate process feedstock sample properties associated with the one or more intermediate process feedstock samples; and (c) the prescriptively controlling is based at least in part on the one or more intermediate process feedstock sample properties.

Clause 35. The method of clause 34, wherein the prescriptively controlling further comprises controlling one or more operating parameters associated with the vacuum distillation unit against operating constraints of the vacuum distillation unit.

Clause 36. A distillation unit control assembly to enhance control of a refining process associated with a petroleum refining operation, the distillation unit control assembly comprising: (a) a first spectroscopic analyzer positioned to: (i) receive a hydrocarbon feedstock sample of a hydrocarbon feedstock supplied to one or more first processing units associated with the petroleum refining operation, the one or more first processing units comprising one or more fractionation units; (ii) analyze the hydrocarbon feedstock sample to provide hydrocarbon feedstock sample spectra; and (iii) predict one or more hydrocarbon feedstock sample properties associated with the hydrocarbon feedstock sample based at least in part on the hydrocarbon feedstock sample spectra; (b) a second spectroscopic analyzer positioned to: (i) receive a unit material sample of one more unit materials produced by the one or more first processing units, the one or more unit materials comprising one or more of intermediate materials or unit product materials, the first spectroscopic analyzer and the second spectroscopic analyzer being calibrated to generate standardized spectral responses; (ii) analyze the unit material sample to provide unit material sample spectra; and (iii) predict one or more unit material sample properties associated with the unit material sample based at least in part on the unit material sample spectra; (c) a sample conditioning assembly positioned to one or more of: (i) condition the hydrocarbon feedstock sample, prior to being supplied to the first spectroscopic analyzer, to one or more of filter the hydrocarbon feedstock sample, change a temperature of the hydrocarbon feedstock sample, dilute in solvent the hydrocarbon feedstock sample, or degas the hydrocarbon feedstock sample; or (ii) condition the unit material sample, prior to being supplied to the second spectroscopic analyzer, to one or more of filter the unit material sample, change a temperature of the unit material sample, dilute in solvent the unit material sample, or degas the unit material sample; and (d) a process controller in communication with the first spectroscopic analyzer and the second spectroscopic analyzer, the process controller being configured to prescriptively control, during the refining process, based at least in part on the one or more hydrocarbon feedstock sample properties and the one or more unit material sample properties, one or more of: (i) one or more hydrocarbon feedstock parameters associated with the hydrocarbon feedstock supplied to the one or more first processing units; (ii) the one or more hydrocarbon feedstock properties associated with the hydrocarbon feedstock supplied to the one or more first processing units; (iii) one or more intermediate properties associated with the intermediate materials produced by the one or more first processing units; (iv) one or more unit materials properties associated with the one or more unit materials; (v) operation of the one or more first processing units; or (vi) operation of one or more second processing units positioned downstream relative to the one or more first processing units, so that the prescriptively controlling during the refining process causes the refining process to produce one or more of: (i) one or more intermediate materials each having one or more properties within a selected range of one or more target properties of the one or more intermediate materials; (ii) one or more unit materials each having one or more properties within a selected range of one or more target properties of the one or more unit materials; or (iii) one or more downstream materials each having one or more properties within a selected range of one or more target properties of the one or more downstream materials, thereby to cause the refining process to achieve material outputs that more accurately and responsively converge on one or more of the target properties.

Clause 37. The distillation unit control assembly of clause 36, wherein: the hydrocarbon feedstock comprises a blended hydrocarbon feedstock including a plurality of hydrocarbon feedstocks from respective hydrocarbon feedstock flows; and the process controller is configured to control one or more feed ratios of the respective hydrocarbon feedstock flows.

Clause 38. The distillation unit control assembly of clause 36, wherein the process controller is configured to prescriptively control one or more of: (i) content of the hydrocarbon feedstock, or (ii) operation of the one or more first processing units.

Clause 39. The distillation unit control assembly of clause 36, wherein the process controller is configured to numerically treat one or more of a spectral response of the first spectroscopic analyzer or a spectral response of the second spectroscopic analyzer.

Clause 40. The distillation unit control assembly of clause 39, wherein the numerically treat comprises treatment via Fourier transformation.

Clause 41. The distillation unit control assembly of clause 36, wherein one or more of the first spectroscopic analyzer or the second spectroscopic analyzer comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer.

Clause 42. The distillation unit control assembly of clause 36, wherein one or more of: (a) the analysis of the hydrocarbon feedstock sample is performed on-line and in real-time; (b) the analysis of the hydrocarbon feedstock sample is performed off-line in a laboratory setting; (c) the analysis of the unit material sample is performed on-line and in real-time; or (d) the analysis of the unit material sample is performed off-line in a laboratory setting.

Clause 43. The distillation unit control assembly of clause 36, wherein the process controller is configured to control one or more of: (a) content of the hydrocarbon feedstock supplied to the one or more first processing units; (b) content of the intermediate materials produced by the one or more first processing units; (c) operation of the one or more first processing units; (d) content of the unit product materials; or (e) operation of the one or more second processing units.

Clause 44. The distillation unit control assembly of clause 36, wherein the process controller is configured to improve an accuracy of predicting one or more of: (a) the one or more hydrocarbon feedstock sample properties associated with the hydrocarbon feedstock sample; (b) the one or more unit material sample properties associated with the unit material sample; (c) content of the hydrocarbon feedstock supplied to the one or more first processing units; (d) content of the one or more intermediate materials produced by the one or more first processing units; (e) content of the unit product materials produced by the one or more first processing units; or (f) content of the downstream materials produced by the one or more of the second processing units.

Clause 45. The distillation unit control assembly of clause 36, wherein the process controller is configured to one or more of: (a) control one or more of the temperature of the hydrocarbon feedstock sample or the temperature of the unit material sample, thereby to substantially maintain the one or more of the temperature of the hydrocarbon feedstock sample or the temperature of the unit material sample within a respective temperature range; or (b) apply one or more temperature correction factors to one or more of: (i) the hydrocarbon feedstock sample spectra; (ii) the one or more hydrocarbon feedstock sample properties; (iii) the unit material sample spectra; or (iv) the one or more unit material properties.

Clause 46. The distillation unit control assembly of clause 36, wherein the process controller is configured to control one or more process parameters, the one or more process parameters comprising one or more of: (a) a flow rate of the hydrocarbon feedstock supplied to the one or more first processing units; (b) one or more feed ratios of a plurality of hydrocarbon feedstock flows combined into the hydrocarbon feedstock; (c) a pressure of the hydrocarbon feedstock supplied to the one or more first processing units; or (d) a preheating temperature of the hydrocarbon feedstock supplied to the one or more first processing units.

Clause 47. The distillation unit control assembly of clause 36, wherein one or more of: (a) the predicting of the one or more hydrocarbon feedstock sample properties comprises predicting a boiling point associated with the hydrocarbon feedstock; or (b) the predicting of the one or more unit material sample properties comprises predicting a boiling point associated with the one or more unit materials.

Clause 48. The distillation unit control assembly of clause 47, wherein the distillation unit control assembly is configured to control, based at least in part on one or more of (a) the boiling point associated with the hydrocarbon feedstock or (b) the boiling point associated with the one or more unit materials, operation of an atmospheric distillation unit.

Clause 49. The distillation unit control assembly of clause 47, wherein the process controller is configured to predict, based at least in part on one or more of (a) the boiling point associated with the hydrocarbon feedstock or (b) the boiling point associated with the one or more unit materials, a yield fraction associated with the one or more unit materials.

Clause 50. The distillation unit control assembly of clause 36, wherein the unit material sample comprises naphtha, and the process controller is configured to control, based at least in part on a boiling point of the naphtha, one or more of: (a) downstream flows of one or more of: (i) intermediate materials associated with the naphtha or (ii) unit product materials associated with the naphtha; (b) downstream flows of one or more of: (i) intermediate materials associated with distillates or (ii) unit product materials associated with distillates; (c) a ratio of downstream flows associated with the naphtha to downstream flows associated with kerosene; or (d) operation of a naphtha stripper.

Clause 51. The distillation unit control assembly of clause 50, wherein the control of the operation of the naphtha stripper comprises adjusting a hydrocarbon stream into the naphtha stripper to improve flash.

Clause 52. The distillation unit control assembly of clause 36, wherein the predict the one or more hydrocarbon feedstock sample properties comprises predicting one or more properties associated with desalter crude water, and the process controller is configured to control, based at least in part on the one or more properties associated with the desalter crude water, operation of an upstream desalter unit.

Clause 53. The distillation unit control assembly of clause 36, wherein the process controller is configured to display, via a display, one or more of: (a) the one or more hydrocarbon feedstock sample properties, or (b) the one or more unit material sample properties.

Clause 54. The distillation unit control assembly of clause 36, wherein the prescriptively control of operation of one or more of: (a) the one or more first processing units, or (b) the one or more second processing units, comprises: comparing one or more of: (a) the one or more hydrocarbon feedstock sample properties or (b) the one or more unit material sample properties, to one or more of: (i) material properties of a material database; (ii) one or more threshold values associated with operation of one or more of: (aa) the one or more first processing units, or (bb) the one or more second processing units; or (iii) target properties.

Clause 55. The distillation unit control assembly of clause 54, wherein the target properties comprise target content associated with one or more of: (a) content of the hydrocarbon feedstock supplied to the one or more first processing units; (b) content of the intermediate materials produced by one or more of the first processing units; (c) content of the unit product materials produced by one or more of the first processing units; or (d) content of the downstream materials produced by one or more of the second processing units.

Clause 56. The distillation unit control assembly of clause 36, wherein: at least one of the one or more first processing units comprises an atmospheric distillation unit; and the one or more unit material sample properties comprise one or more of: (a) a quantitative composition of a hydrocarbon feedstock including a mixture of one or more feedstocks; (b) densities and viscosities of products of the atmospheric distillation unit; or (c) one or more of: (i) paraffinic content or (ii) aromatic content, of products of the atmospheric distillation unit.

Clause 57. The distillation unit control assembly of clause 36, wherein: at least one of the one or more first processing units comprises an atmospheric distillation unit; and the one or more unit material sample properties are comprised of one or more of: API gravity, UOP K factor, distillation points, Coker gas oil content, carbon residue content, nitrogen content, sulfur content, saturates content, thiophene content, single-ring aromatics content, dual-ring aromatics content, triple-ring aromatics content, or quad-ring aromatics content.

Clause 58. The distillation unit control assembly of clause 36, wherein the one or more hydrocarbon feedstock sample properties and the one or more unit material sample properties comprise a content ratio indicative of relative amounts of one or more hydrocarbon classes present in one or more of (a) the hydrocarbon feedstock sample or (b) the unit material sample.

Clause 59. The distillation unit control assembly of clause 36, wherein the one or more unit material sample properties are comprised of one or more of: an amount of butane-free gasoline, an amount of total butane, an amount of dry gas, an amount of coke, an amount of gasoline, octane rating, an amount of light fuel oil, an amount of heavy fuel oil, an amount of hydrogen sulfide, an amount of sulfur in light fuel oil, or an aniline point of light fuel oil.

Clause 60. The distillation unit control assembly of clause 36, wherein: the one or more unit material sample properties are comprised of one or more of: pentane content, raw crude water content, desalted crude water content, heavy atmospheric gas oil (HAGO) content, light atmospheric gas oil (LAGO) flash, or kerosene flash point; and the process controller is configured to control, based at least in part on the one or more of the pentane content, the raw crude water content, the desalted crude water content, the HAGO content, the LAGO flash, or the kerosene flash point, one or more of: crude blend, make-up water, desalter severity, HAGO wash rate, stripping, LAGO draw rate, stripping steam, or kerosene draw of the one or more of the first processing units.

Clause 61. The distillation unit control assembly of clause 36, wherein: the one or more unit material sample properties are comprised of one or more of: ethane content, propane content, propene content, isobutane content, or n-butane content; and the process controller is configured to control, based at least in part on the one or more of the ethane content, the propane content, the propene content, the isobutane content, or the n-butane content, one or more of: absorber pressure, lean oil flow rate, lean oil temperature, high-pressure separator temperature, reactor conversion, or stripper reboiler duty.

Clause 62. The distillation unit control assembly of clause 36, wherein: the one or more unit material sample properties are comprised of one or more of high-pressure separator water content or stripper bottoms water content; and the process controller is configured to control, based at least in part on the one or more of the high-pressure separator water content or the stripper bottoms water content, a temperature of a high-pressure separator.

Clause 63. The distillation unit control assembly of clause 36, wherein: (a) the one or more first processing units comprises an atmospheric distillation unit comprising an atmospheric column; (b) the process controller is configured to control one or more of: (i) supplying the hydrocarbon feedstock to the atmospheric column; (ii) separating, via the atmospheric column, the hydrocarbon feedstock into a plurality of unit materials; (iii) separating, via a side stripper, at least one of the plurality of unit materials from the atmospheric column; (iv) separating one or more from one or more locations of the atmospheric column; (v) separating one or more unit material samples from the at least one of the plurality of unit materials; (vi) analyzing, via one or more of the first spectroscopic analyzer, the second spectroscopic analyzer, or one or more additional spectroscopic analyzers, the one or more intermediate samples; (vii) predicting, based at least in part on the analyzing of the one or more intermediate samples, one or more intermediate sample properties associated with the one or more intermediate samples; (viii) analyzing, via one or more of the first spectroscopic analyzer, the second spectroscopic analyzer, or the one or more additional spectroscopic analyzers, the one or more unit material samples; and (ix) predicting, based at least in part in the analyzing of the one or more unit material samples, one or more unit material sample properties associated with the one or more unit material samples; and (c) the prescriptively control is based at least in part on one or more of: (i) the one or more intermediate sample properties, or (ii) the one or more unit material sample properties.

Clause 64. The distillation unit control assembly of clause 63, wherein the process controller is configured to control supply of one or more of the plurality of unit materials to a saturated gas unit associated with the atmospheric distillation unit.

Clause 65. The distillation unit control assembly of clause 63, wherein the atmospheric distillation unit further comprises a heat exchanger and a return conduit, and the process controller is configured to control one or more of: changing, via the heat exchanger, a temperature of at least one of the unit materials, thereby to provide a temperature-controlled unit materials stream; and returning, via the return conduit, at least a portion of the temperature-controlled unit materials stream to the atmospheric column.

Clause 66. The distillation unit control assembly of clause 63, wherein the process controller is configured to control, prior to the analyzing the one or more intermediate samples, conditioning of the one or more intermediate samples, thereby to provide one or more conditioned intermediate samples; and wherein the process controller is configured to control, prior to the analyzing of the one or more unit material samples, conditioning of the one or more unit material samples, thereby to provide one or more conditioned unit material samples.

Clause 67. The distillation unit control assembly of clause 63, wherein the hydrocarbon feedstock comprises a blended hydrocarbon feedstock including a plurality of hydrocarbon feedstocks from respective hydrocarbon feedstock flows; wherein the process controller is further configured to: (i) analyze the content of one or more of the plurality of hydrocarbon feedstocks; and (ii) predict, based at least in part on the content of one or more of the plurality of hydrocarbon feedstocks, one or more of: (a) volume percent yield of fractionated rundown streams of the refining process; or (b) yields of finished products of the refining process.

Clause 68. The distillation unit control assembly of clause 63, wherein the process controller is configured to control one or more operating parameters associated with the atmospheric distillation unit against operating constraints of the atmospheric distillation unit.

Clause 69. The distillation unit control assembly of clause 63, wherein: (a) the one or more first processing units further comprise a vacuum distillation unit including a vacuum column positioned to receive a residue stream from the atmospheric column; (b) the process controller is configured to control one or more of: (i) separating, via the vacuum column, the residue stream into one or more intermediate process feedstocks; (ii) separating one or more intermediate process feedstock samples from the one or more intermediate process feedstocks; (iii) analyzing, via one or more of the first spectroscopic analyzer, the second spectroscopic analyzer, or the one or more additional spectroscopic analyzers, the one or more intermediate process feedstock samples; and (iv) predicting, based at least in part on the analyzing of the one or more intermediate process feedstock samples, one or more intermediate process feedstock sample properties associated with the one or more intermediate process feedstock samples; and (c) the prescriptively controlling is based at least in part on the one or more intermediate process feedstock sample properties.

Clause 70. The distillation unit control assembly of clause 69, wherein the process controller is configured to control one or more operating parameters associated with the vacuum distillation unit against operating constraints of the vacuum distillation unit.

Clause 71. A distillation unit control assembly to enhance control of a refining process associated with a petroleum refining operation, the distillation unit control assembly being in communication with one or more spectroscopic analyzers and one or more first processing units including one or more of fractionation units, the distillation unit control assembly being configured to: predict one or more hydrocarbon feedstock sample properties associated with a hydrocarbon feedstock sample based at least in part on hydrocarbon feedstock sample spectra generated by the one or more spectroscopic analyzers; predict one or more unit material sample properties associated with a unit material sample based at least in part on unit material sample spectra generated by the one or more spectroscopic analyzers, the one or more unit material sample properties being associated with one or more unit materials produced by the one or more first processing units and the one or more unit materials including one or more of intermediate materials or unit product materials; and prescriptively control, during the refining process, based at least in part on the one or more hydrocarbon feedstock sample properties, and the one or more unit material sample properties, one or more of: (a) the one or more hydrocarbon feedstock properties associated with hydrocarbon feedstock supplied to the one or more first processing units; (b) one or more intermediate properties associated with the intermediate materials produced by one or more of the first processing units; (c) operation of the one or more first processing units; (d) one or more unit materials properties associated with the one or more unit materials produced by the one or more first processing units; or (e) operation of one or more second processing units positioned downstream relative to the one or more first processing units, so that the prescriptively controlling causes the refining process to produce one or more of: (i) one or more intermediate materials each having one or more properties within a selected range of one or more target properties of the one or more intermediate materials; (ii) one or more unit materials each having one or more properties within a selected range of one or more target properties of the one or more unit materials; or (iii) one or more downstream materials each having one or more properties within a selected range of one or more target properties of the one or more downstream materials, thereby to cause the refining process to achieve material outputs that more accurately and responsively converge on one or more of the target properties.

Clause 72. The distillation unit control assembly of clause 71, wherein the distillation unit control assembly is configured to prescriptively control, during the refining process, one or more operating parameters of the one or more first processing units against operating constraints associated with the one or more first processing units.

Clause 73. The distillation unit control assembly of clause 71, wherein the prescriptively controlling comprises controlling one or more process parameters, the one or more process parameters comprising one or more of: (i) one or more hydrocarbon feedstock parameters; (ii) a rate of supply of the hydrocarbon feedstock to the one or more first processing units; (iii) a pressure of the hydrocarbon feedstock supplied to the one or more first processing units; (iv) a preheating temperature of the hydrocarbon feedstock supplied to the one or more first processing units; (v) one or more unit materials parameters; (vi) a rate of supply of the one or more unit materials to the one or more second processing units; (vii) a pressure of the one or more unit materials supplied to the one or more second processing units; (viii) a preheating temperature of the one or more unit materials supplied to the one or more second processing units; (ix) a temperature in the one or more first processing units; (x) a pressure in the one or more first processing units; (xi) a temperature in the one or more second processing units; or (xii) a pressure in the one or more second processing units.

Clause 74. The distillation unit control assembly of clause 71, wherein one or more of: (a) the one or more hydrocarbon feedstock properties associated with the hydrocarbon feedstock supplied to the one or more first processing units are comprised of one or more of API gravity, UOP K factor, distillation points, Coker gas oil content, carbon residue content, nitrogen content, sulfur content, catalyst oil ratio, saturates content, thiophene content, single-ring aromatics content, dual-ring aromatics content, triple-ring aromatics content, or quad-ring aromatics content; or (b) the one or more hydrocarbon feedstock sample properties and the one or more unit material sample properties comprise a content ratio indicative of relative amounts of one or more hydrocarbon classes present in one or more of the hydrocarbon feedstock sample or the unit material sample.

Clause 75. The distillation unit control assembly of clause 71, wherein the one or more unit material sample properties associated with the one or more unit materials are comprised of one or more of pentane content, raw crude water content, desalted crude water content, heavy atmospheric gas oil (HAGO) content, light atmospheric gas oil (LAGO) flash, or kerosene flash point.

Clause 76. A distillation unit control assembly for performing a refining process associated with a petroleum refining operation, the distillation unit control assembly comprising: (a) one or more first processing units associated with the petroleum refining operation, the one or more first processing units including one or more fractionation units; (b) a first spectroscopic analyzer positioned to: (i) receive, during the refining process, a hydrocarbon feedstock sample of a hydrocarbon feedstock supplied to the one or more first processing units, the hydrocarbon feedstock having one or more hydrocarbon feedstock properties; (ii) analyze, during the refining process, the hydrocarbon feedstock sample to provide hydrocarbon feedstock sample spectra; and (iii) predict, during the refining process, one or more hydrocarbon feedstock sample properties associated with the hydrocarbon feedstock sample based at least in part on the hydrocarbon feedstock sample spectra; (c) a second spectroscopic analyzer positioned to: (i) receive, during the refining process, a unit material sample of one more unit materials produced by the one or more first processing units, the one or more unit materials including one or more of intermediate materials or unit product materials, the first spectroscopic analyzer and the second spectroscopic analyzer being calibrated to generate standardized spectral responses; (ii) analyze, during the refining process, the unit material sample to provide unit material sample spectra; and (iii) predict, during the refining process, one or more unit material sample properties associated with the unit material sample based at least in part on the unit material sample spectra; (d) a sample conditioning assembly positioned to one or more of: (i) condition the hydrocarbon feedstock sample, prior to being supplied to the first spectroscopic analyzer, to one or more of filter the hydrocarbon feedstock sample, change a temperature of the hydrocarbon feedstock sample, or degas the hydrocarbon feedstock sample; or (ii) condition the unit material sample, prior to being supplied to the second spectroscopic analyzer, to one or more of filter the unit material sample, change a temperature of the unit material sample, or degas the unit material sample; and (e) a process controller in communication with the first spectroscopic analyzer and the second spectroscopic analyzer during the refining process, the process controller configured to prescriptively control, during the refining process, based at least in part on the one or more hydrocarbon feedstock properties and the one or more unit material sample properties, one or more of: (i) one or more hydrocarbon feedstock parameters associated with the hydrocarbon feedstock supplied to the one or more first processing units; (ii) one or more intermediates properties associated with the intermediate materials produced by one or more of the first processing units; (iii) operation of the one or more first processing units; (iv) one or more unit materials properties associated with the one or more unit product materials; or (v) operation of one or more second processing units positioned downstream relative to the one or more first processing units, so that the prescriptively controlling during the refining process causes the refining process to produce one or more of: (aa) one or more intermediate materials each having one or more properties within a selected range of one or more target properties of the one or more intermediate materials; (bb) one or more unit product materials each having one or more properties within a selected range of one or more target properties of the one or more unit product materials; or (cc) one or more downstream materials each having one or more properties within a selected range of one or more target properties of the one or more downstream materials, thereby to cause the refining process to achieve material outputs that more accurately and responsively converge on one or more of the target properties.

Clause 77. The distillation unit control assembly of clause 76, wherein the refining process comprises a distillation process.

Clause 78. The distillation unit control assembly of clause 76, wherein the sample conditioning assembly comprises one or more of: one or more filters including filter media configured to remove one or more of water, particulates, or other contaminants from one or more of the hydrocarbon feedstock sample or the unit material sample; a temperature control unit configured to change a temperature of one or more of the hydrocarbon feedstock sample or the unit material sample to provide a temperature-adjusted sample stream of the one or more of the hydrocarbon feedstock sample or the unit material sample; or a degassing unit configured to degas one or more of the hydrocarbon feedstock sample or the unit material sample to provide a degassed sample stream of the one or more of the hydrocarbon feedstock sample or the unit material sample.

Clause 79. The distillation unit control assembly of clause 76, wherein: the hydrocarbon feedstock comprises a blended hydrocarbon feedstock including a plurality of hydrocarbon feedstocks from respective hydrocarbon feedstock flows; and the process controller is configured to control one or more feed ratios of the respective hydrocarbon feedstock flows.

Clause 80. The distillation unit control assembly of clause 76, wherein the process controller is configured to control one or more of: (i) content of the hydrocarbon feedstock, or (ii) operation of the one or more first processing units.

Clause 81. The distillation unit control assembly of clause 76, wherein the process controller is further configured to control content of the one or more downstream materials via control of one or more of: (a) content of the hydrocarbon feedstock supplied to the one or more first processing units; (b) the operation of the one or more first processing units; (c) content of the intermediate materials produced by the one or more first processing units; (d) content of the unit product materials; or (e) the operation of the one or more second processing units.

Clause 82. The distillation unit control assembly of clause 76, wherein the process controller is configured to operate a prescriptive analytical model configured to improve an accuracy of prediction of one or more of: (a) the one or more hydrocarbon feedstock sample properties associated with the hydrocarbon feedstock sample; (b) the one or more unit material sample properties associated with the unit material sample; (c) content of the hydrocarbon feedstock supplied to the one or more first processing units; (d) content of the one or more intermediate materials produced by the one or more first processing units; (e) content of the unit product materials produced by the one or more first processing units; or (f) content of the downstream materials produced by the one or more of the second processing units.

Clause 83. The distillation unit control assembly of clause 76, wherein the process controller is configured to one or more of: (a) control one or more of the temperature of the hydrocarbon feedstock sample or the temperature of the unit material sample, thereby to substantially maintain the one or more of the temperature of the hydrocarbon feedstock sample or the temperature of the unit material sample within respective selected temperature ranges; or (b) apply one or more temperature correction factors to one or more of: (i) the hydrocarbon feedstock sample spectra; (ii) the one or more hydrocarbon feedstock sample properties; (iii) the unit material sample spectra; or (iv) the one or more unit material properties.

Clause 84. The distillation unit control assembly of clause 76, wherein the process controller is configured to control one or more process parameters, the one or more process parameters comprising one or more of: (a) a flow rate of the hydrocarbon feedstock supplied to the one or more first processing units; (b) one or more feed ratios of a plurality of hydrocarbon feedstock flows combined into the hydrocarbon feedstock; (c) a pressure of the hydrocarbon feedstock supplied to the one or more first processing units; or (d) a preheating temperature of the hydrocarbon feedstock supplied to the one or more first processing units.

Clause 85. The distillation unit control assembly of clause 84, wherein the process controller is configured to control, based at least in part on one or more of (a) a boiling point associated with the hydrocarbon feedstock or (b) a boiling point associated with the one or more unit materials, the operation of an atmospheric distillation unit.

Clause 86. The distillation unit control assembly of clause 85, wherein the process controller is configured to predict, based at least in part on one or more of (a) the boiling point associated with the hydrocarbon feedstock or (b) the boiling point associated with the one or more unit materials, a yield fraction associated with the one or more unit materials.

Clause 87. The distillation unit control assembly of clause 76, wherein the unit material sample comprises naphtha, and the process controller is configured to control, based at least in part on a boiling point of the naphtha, one or more of: (a) downstream flows of one or more of intermediate materials associated with the naphtha or unit product materials associated with the naphtha; (b) downstream flows of one or more of intermediate materials associated with distillates or unit product materials associated with the distillates; (c) a ratio of downstream flows associated with the naphtha to downstream flows associated with kerosene; or (d) operation of a naphtha stripper.

Clause 88. The distillation unit control assembly of clause 87, wherein the process controller is configured to adjust one or more of (a) the one or more hydrocarbon feedstock properties of the hydrocarbon feedstock or (b) the one or more unit material sample properties associated with the unit materials, to improve flash.

Clause 89. The distillation unit control assembly of clause 76, wherein the process controller is configured to predict one or more properties associated with desalter crude water, and control, based at least in part on the one or more properties associated with the desalter crude water, operation of an upstream desalter unit.

Clause 90. The distillation unit control assembly of clause 76, wherein: at least one of the one or more first processing units comprises an atmospheric distillation unit; and the one or more hydrocarbon feedstock sample properties and the one or more unit material sample properties comprise one or more of: (a) a quantitative composition of a hydrocarbon feedstock including a mixture of one or more feedstocks; (b) densities and viscosities of unit materials of the atmospheric distillation unit; or (c) one or more of (i) paraffinic content or (ii) aromatic content, of unit materials of the atmospheric distillation unit.

Clause 91. The distillation unit control assembly of clause 76, wherein: the one or more unit material sample properties are comprised of one or more of: ethane content, propane content, propene content, isobutane content, or n-butane content; and the process controller is configured to control, based at least in part on the one or more of the ethane content, the propane content, the propene content, the isobutane content, or the n-butane content, one or more of: absorber pressure, lean oil flow rate, lean oil temperature, high-pressure separator temperature, reactor conversion, or stripper reboiler duty.

Clause 92. The distillation unit control assembly of clause 76, wherein: the one or more unit material sample properties are comprised of one or more of high-pressure separator water content or stripper bottoms water content; and the process controller is configured to control, based at least in part on the one or more of the high-pressure separator water content or the stripper bottoms water content, a temperature of a high-pressure separator.

Clause 93. The distillation unit control assembly of clause 76, wherein: (a) the one or more first processing units comprise an atmospheric distillation unit including an atmospheric column and one or more side strippers, the atmospheric column being configured to separate the hydrocarbon feedstock into unit materials comprising a plurality of distillation products, and the one or more side strippers being configured to separate at least one of the plurality of distillation products from the atmospheric column; (b) the process controller is configured to predict one or more material properties associated with one or more of the plurality of distillation products separated from one or more locations of the atmospheric column; and (c) the prescriptively controlling is based at least in part on the one or more material properties of the one or more of the plurality of distillation products.

Clause 94. The distillation unit control assembly of clause 93, wherein the prescriptively controlling comprises controlling supply of the one or more of the plurality of the distillation products to a saturated gas unit associated with the crude distillation unit.

Clause 95. The distillation unit control assembly of clause 93, wherein the atmospheric distillation unit comprises: a heat exchanger configured to change a temperature of at least one of the plurality of distillation products, thereby to provide a temperature-controlled distillation product; and a return conduit positioned to return at least a portion of the temperature-controlled distillation product to the atmospheric column.

Clause 96. The distillation unit control assembly of clause 93, wherein the process controller is configured to control one or more operating parameters associated with the atmospheric distillation unit against operating constraints of the atmospheric distillation unit.

Clause 97. The distillation unit control assembly of clause 93, wherein: (a) the one or more first processing units further comprises a vacuum distillation unit including a vacuum column configured to separate the residue stream into one or more unit materials, the one or more unit materials comprising vacuum distillation products; (b) the process controller is configured to predict one or more vacuum distillation product sample properties associated with the one or more vacuum distillation products; and (c) the prescriptively controlling is based at least in part on the one or more vacuum distillation product sample properties.

Clause 98. The distillation unit control assembly of clause 97, wherein the process controller is configured to control one or more operating parameters associated with the vacuum distillation unit against operating constraints of the vacuum distillation unit.

This is a continuation-in-part of U.S. Non-Provisional application Ser. No. 17/652,431, filed Feb. 24, 2022, titled “METHODS AND ASSEMBLIES FOR DETERMINING AND USING STANDARDIZED SPECTRAL RESPONSES FOR CALIBRATION OF SPECTROSCOPIC ANALYZERS,” which claims priority to and the benefit of U.S. Provisional Application No. 63/153,452, filed Feb. 25, 2021, titled “METHODS AND ASSEMBLIES FOR DETERMINING AND USING STANDARDIZED SPECTRAL RESPONSES FOR CALIBRATION OF SPECTROSCOPIC ANALYZERS,” and U.S. Provisional Application No. 63/268,456, filed Feb. 24, 2022, titled “ASSEMBLIES AND METHODS FOR ENHANCING CONTROL OF FLUID CATALYTIC CRACKING (FCC) PROCESSES USING SPECTROSCOPIC ANALYZERS,” the disclosures of all of which are incorporated herein by reference in their entireties.

Furthermore, the scope of the present disclosure shall be construed to cover various modifications, combinations, additions, alterations, etc., above and to the above-described embodiments, which shall be considered to be within the scope of this disclosure. Accordingly, various features and characteristics as discussed herein may be selectively interchanged and applied to other illustrated and non-illustrated embodiment, and numerous variations, modifications, and additions further can be made thereto without departing from the spirit and scope of the present disclosure as set forth in the appended claims.

What is claimed is:

1. A method for enhancing control of a refining process associated with a petroleum refining operation, the method comprising:
conditioning a hydrocarbon feedstock sample of a hydrocarbon feedstock supplied to one or more first processing units associated with the petroleum refining operation, the conditioning including one or more of: (a) filtering the hydrocarbon feedstock sample, (b) changing a temperature of the hydrocarbon feedstock sample, (c) diluting the hydrocarbon feedstock sample in solvent, or (d) degassing the hydrocarbon feedstock sample, the hydrocarbon feedstock having one or more hydrocarbon feedstock properties and the one or more first processing units including one or more fractionation units;
analyzing the hydrocarbon feedstock sample via a first spectroscopic analyzer to provide hydrocarbon feedstock sample spectra;
predicting one or more hydrocarbon feedstock sample properties associated with the hydrocarbon feedstock sample based at least in part on the hydrocarbon feedstock sample spectra;
operating the one or more first processing units to produce one or more unit materials, the one or more unit materials having one or more unit materials properties, and the one or more unit materials comprising one or more of intermediate materials or unit product materials;
conditioning a unit material sample from the one or more unit materials to one or more of: (a) filter the unit material sample, (b) change a temperature of the unit material sample, (c) dilute the unit material sample in solvent, or (d) degas the unit material sample;
analyzing the unit material sample via one or more of the first spectroscopic analyzer or a second spectroscopic analyzer to provide unit material sample spectra, the one or more of the first spectroscopic analyzer or the second spectroscopic analyzer being calibrated to generate standardized spectral responses and wherein one or more of the first spectroscopic analyzer or the second spectroscopic analyzer comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer;
predicting one or more unit material sample properties associated with the unit material sample based at least in part on the unit material sample spectra; and
prescriptively controlling, during the refining process, via one or more controllers based at least in part on the one or more hydrocarbon feedstock sample properties and the one or more unit material sample properties, one or more of:
(a) one or more hydrocarbon feedstock properties associated with the hydrocarbon feedstock supplied to the one or more first processing units;
(b) one or more intermediate properties associated with the intermediate materials produced by the one or more first processing units;
(c) one or more unit product materials properties associated with the unit product materials;
(d) operation of the one or more first processing units; or
(e) operation of one or more second processing units positioned downstream relative to the one or more first processing units,
so that the prescriptively controlling, during the refining process, causes the refining process to produce one or more of:
(i) one or more intermediate materials each having one or more properties within a range of one or more target properties of the one or more intermediate materials,
(ii) one or more unit product materials each having one or more properties within a range of one or more target properties of the one or more unit product materials, or
(iii) one or more downstream materials each having one or more properties within a range of one or more target properties of the one or more downstream materials,
thereby to cause the refining process to achieve material outputs that more accurately and responsively converge on one or more of the target properties.

2. The method of claim 1, wherein:
the hydrocarbon feedstock comprises a blended hydrocarbon feedstock including a plurality of hydrocarbon feedstocks from respective hydrocarbon feedstock flows; and
the prescriptively controlling the one or more hydrocarbon feedstock properties associated with the hydrocarbon feedstock supplied to the one or more first processing units comprises controlling one or more feed ratios of the respective hydrocarbon feedstock flows.

3. The method of claim 1, wherein the prescriptively controlling of one or more of (a) the one or more intermediate properties associated with the intermediate materials, or (b) the one or more unit product materials properties associated with the unit product materials, comprises:
controlling one or more of: (i) content of the hydrocarbon feedstock, or (ii) operation of the one or more first processing units.

4. The method of claim 1, wherein the one or more fractionation units comprise one or more of an atmospheric distillation unit, a vacuum distillation unit, a fracking oil fractionation unit, a saturated gas unit, or a condensate fractionation unit.

5. The method of claim 1, wherein one or more of:
(a) the predicting of the one or more hydrocarbon feedstock properties comprises predicting a boiling point associated with the hydrocarbon feedstock sample, and the method comprises controlling, based at least in part on the boiling point associated with the hydrocarbon feedstock sample, operation of one or more of the one or more first processing units or the one or more second processing units; or
(b) the predicting of the one or more unit material sample properties comprises predicting a boiling point associated with the unit material sample, and the method comprises controlling, based at least in part on the boiling point associated with the unit material sample, operation of one or more of the one or more first processing units or the one or more second processing units.

6. The method of claim 1, further comprising prescriptively controlling content of the one or more downstream materials via control of one or more of:
(a) content of the hydrocarbon feedstock supplied to the one or more first processing units;
(b) the operation of the one or more first processing units;
(c) content of the intermediate materials produced by the one or more first processing units;
(d) content of the unit product materials; or
(e) the operation of the one or more second processing units.

7. The method of claim 1, further comprising one or more of:
(a) prescriptively controlling one or more of the temperature of the hydrocarbon feedstock sample or the temperature of the unit material sample, thereby to substantially maintain the one or more of the temperature of the hydrocarbon feedstock sample or the temperature of the unit material sample within a respective temperature range; or
(b) applying one or more temperature correction factors to one or more of:
(i) the hydrocarbon feedstock sample spectra;
(ii) the one or more hydrocarbon feedstock sample properties;
(iii) the unit material sample spectra; or
(iv) the one or more unit material properties.

8. The method of claim 1, wherein the prescriptively controlling comprises controlling one or more process parameters, the one or more process parameters comprising one or more of:
(a) a flow rate of the hydrocarbon feedstock supplied to the one or more first processing units;
(b) one or more feed ratios of a plurality of hydrocarbon feedstock flows combined into the hydrocarbon feedstock;
(c) a pressure of the hydrocarbon feedstock supplied to the one or more first processing units; or
(d) a preheating temperature of the hydrocarbon feedstock supplied to the one or more first processing units.

9. The method of claim 1, wherein one or more of:
(a) the predicting of the one or more hydrocarbon feedstock sample properties comprises predicting a boiling point associated with the hydrocarbon feedstock; or
(b) the predicting of the one or more unit material sample properties comprises predicting a boiling point associated with the one or more unit materials.

10. The method of claim 9, further comprising controlling, based at least in part on one or more of: (a) the boiling point associated with the hydrocarbon feedstock or (b) the boiling point associated with the one or more unit materials, operation of the one or more fractionation units.

11. The method of claim 1, wherein the unit material sample comprises naphtha, and the method further comprises controlling, based at least in part on a boiling point of the naphtha, one or more of:
(a) downstream flows of one or more of intermediate materials associated with the naphtha or unit product materials associated with the naphtha;
(b) downstream flows of one or more of intermediate materials associated with distillates or unit product materials associated with the distillates;
(c) a ratio of downstream flows associated with the naphtha to downstream flows associated with kerosene; or
(d) operation of a naphtha stripper.

12. The method of claim 1, wherein the predicting of the one or more hydrocarbon feedstock sample properties comprises predicting one or more properties associated with desalter crude water, and the method further comprises controlling, based at least in part on the one or more properties associated with the desalter crude water, operation of an upstream desalter unit.

13. The method of claim 1, wherein:
at least one of the one or more first processing units comprises an atmospheric distillation unit; and
the one or more hydrocarbon feedstock sample properties and the one or more unit material sample properties are comprised of one or more of:
(a) a quantitative composition of a hydrocarbon feedstock including a mixture of one or more feedstocks;
(b) densities of products of the atmospheric distillation unit and viscosities of the products of the atmospheric distillation unit; or
(c) one or more of (i) paraffinic content or (ii) aromatic content, of products of the atmospheric distillation unit.

14. The method of claim 1, wherein:
the one or more unit material sample properties comprises one or more of: ethane content, propane content, propene content, isobutane content, or n-butane content; and
the method further comprises controlling, based at least in part on the one or more of the ethane content, the propane content, the propene content, the isobutane content, or the n-butane content, one or more of: absorber pressure, lean oil flow rate, lean oil temperature, high-pressure separator temperature, reactor conversion, or stripper reboiler duty.

15. A distillation unit control assembly to enhance control of a refining process associated with a petroleum refining operation, the distillation unit control assembly comprising:
(a) a first spectroscopic analyzer positioned to:
(i) receive a hydrocarbon feedstock sample of a hydrocarbon feedstock supplied to one or more first processing units associated with the petroleum refining operation, the one or more first processing units comprising one or more fractionation units;
(ii) analyze the hydrocarbon feedstock sample to provide hydrocarbon feedstock sample spectra; and
(iii) predict one or more hydrocarbon feedstock sample properties associated with the hydrocarbon feedstock sample based at least in part on the hydrocarbon feedstock sample spectra;
(b) a second spectroscopic analyzer, wherein one or more of the first spectroscopic analyzer or the second spectroscopic analyzer comprises one of a near-infrared spectroscopic analyzer, a mid-infrared spectroscopic analyzer, a combination of a near-infrared spectroscopic analyzer and a mid-infrared spectroscopic analyzer, a Raman spectroscopic analyzer, or a nuclear magnetic resonance spectroscopic analyzer, positioned to:
(i) receive a unit material sample of one more unit materials produced by the one or more first processing units, the one or more unit materials comprising one or more of intermediate materials or unit product materials, the first spectroscopic analyzer and the second spectroscopic analyzer being calibrated to generate standardized spectral responses;
(ii) analyze the unit material sample to provide unit material sample spectra; and
(iii) predict one or more unit material sample properties associated with the unit material sample based at least in part on the unit material sample spectra;
(c) a sample conditioning assembly positioned to one or more of:
(i) condition the hydrocarbon feedstock sample, prior to being supplied to the first spectroscopic analyzer, to one or more of filter the hydrocarbon feedstock sample, change a temperature of the hydrocarbon feedstock sample, dilute in solvent the hydrocarbon feedstock sample, or degas the hydrocarbon feedstock sample; or
(ii) condition the unit material sample, prior to being supplied to the second spectroscopic analyzer, to one or more of filter the unit material sample, change a temperature of the unit material sample, dilute in solvent the unit material sample, or degas the unit material sample; and
(d) a process controller in communication with the first spectroscopic analyzer and the second spectroscopic analyzer, the process controller being configured to prescriptively control, during the refining process, based at least in part on the one or more hydrocarbon feedstock sample properties and the one or more unit material sample properties, one or more of:
(i) one or more hydrocarbon feedstock parameters associated with the hydrocarbon feedstock supplied to the one or more first processing units;
(ii) the one or more hydrocarbon feedstock properties associated with the hydrocarbon feedstock supplied to the one or more first processing units;
(iii) one or more intermediate properties associated with the intermediate materials produced by the one or more first processing units;
(iv) one or more unit materials properties associated with the one or more unit materials;
(v) operation of the one or more first processing units; or
(vi) operation of one or more second processing units positioned downstream relative to the one or more first processing units,
so that the prescriptively controlling during the refining process causes the refining process to produce one or more of:
(i) one or more intermediate materials each having one or more properties within a selected range of one or more target properties of the one or more intermediate materials;
(ii) one or more unit materials each having one or more properties within a selected range of one or more target properties of the one or more unit materials; or
(iii) one or more downstream materials each having one or more properties within a selected range of one or more target properties of the one or more downstream materials,
thereby to cause the refining process to achieve material outputs that more accurately and responsively converge on one or more of the target properties.

16. The distillation unit control assembly of claim 15, wherein:
the hydrocarbon feedstock comprises a blended hydrocarbon feedstock including a plurality of hydrocarbon feedstocks from respective hydrocarbon feedstock flows; and
the process controller is configured to control one or more feed ratios of the respective hydrocarbon feedstock flows.

17. The distillation unit control assembly of claim 15, wherein the unit material sample comprises naphtha, and the process controller is configured to control, based at least in part on a boiling point of the naphtha, one or more of:
(a) downstream flows of one or more of: (i) intermediate materials associated with the naphtha or (ii) unit product materials associated with the naphtha;
(b) downstream flows of one or more of: (i) intermediate materials associated with distillates or (ii) unit product materials associated with distillates;
(c) a ratio of downstream flows associated with the naphtha to downstream flows associated with kerosene; or
(d) operation of a naphtha stripper.

18. The distillation unit control assembly of claim 17, wherein the control of the operation of the naphtha stripper comprises adjusting a hydrocarbon stream into the naphtha stripper to improve flash.

19. The distillation unit control assembly of claim 15, wherein the process controller being configured to prescriptively control of operation of one or more of: (a) the one or more first processing units, or (b) the one or more second processing units, comprises:
comparing one or more of: (a) the one or more hydrocarbon feedstock sample properties or (b) the one or more unit material sample properties, to one or more of:
(i) material properties of a material database;
(ii) one or more threshold values associated with operation of one or more of: (aa) the one or more first processing units, or (bb) the one or more second processing units; or
(iii) target properties.

20. The distillation unit control assembly of claim 19, wherein the target properties comprise target content associated with one or more of:
(a) content of the hydrocarbon feedstock supplied to the one or more first processing units;
(b) content of the intermediate materials produced by one or more of the first processing units;
(c) content of the unit product materials produced by one or more of the first processing units; or
(d) content of the downstream materials produced by one or more of the second processing units.

21. The distillation unit control assembly of claim 15, wherein:
at least one of the one or more first processing units comprises an atmospheric distillation unit; and
the one or more unit material sample properties comprise one or more of:
(a) a quantitative composition of a hydrocarbon feedstock including a mixture of one or more feedstocks;
(b) densities and viscosities of products of the atmospheric distillation unit; or
(c) one or more of: (i) paraffinic content or (ii) aromatic content, of products of the atmospheric distillation unit.

22. The distillation unit control assembly of claim 15, wherein:
(a) the one or more first processing units comprises an atmospheric distillation unit comprising an atmospheric column;
(b) the process controller is configured to control one or more of:
(i) supplying the hydrocarbon feedstock to the atmospheric column;
(ii) separating, via the atmospheric column, the hydrocarbon feedstock into a plurality of unit materials;
(iii) separating, via a side stripper, at least one of the plurality of unit materials from the atmospheric column;
(iv) separating one or more from one or more locations of the atmospheric column;
(v) separating one or more unit material samples from the at least one of the plurality of unit materials;
(vi) analyzing, via one or more of the first spectroscopic analyzer, the second spectroscopic analyzer, or one or more additional spectroscopic analyzers, the one or more intermediate samples;
(vii) predicting, based at least in part on the analyzing of the one or more intermediate samples, one or more intermediate sample properties associated with the one or more intermediate samples;
(viii) analyzing, via one or more of the first spectroscopic analyzer, the second spectroscopic analyzer, or the one or more additional spectroscopic analyzers, the one or more unit material samples; and
(ix) predicting, based at least in part in the analyzing of the one or more unit material samples, one or more unit material sample properties associated with the one or more unit material samples; and
(c) the prescriptively control is based at least in part on one or more of: (i) the one or more intermediate sample properties, or (ii) the one or more unit material sample properties.

23. The distillation unit control assembly of claim 22, wherein the process controller is configured to control supply of one or more of the plurality of unit materials to a saturated gas unit associated with the atmospheric distillation unit.

24. The distillation unit control assembly of claim 22, wherein the atmospheric distillation unit further comprises a heat exchanger and a return conduit, and the process controller is configured to control one or more of:
changing, via the heat exchanger, a temperature of at least one of the unit materials, thereby to provide a temperature-controlled unit materials stream; and
returning, via the return conduit, at least a portion of the temperature-controlled unit materials stream to the atmospheric column.

25. The distillation unit control assembly of claim 22, wherein the hydrocarbon feedstock comprises a blended hydrocarbon feedstock including a plurality of hydrocarbon feedstocks from respective hydrocarbon feedstock flows;
wherein the process controller is further configured to:
(i) analyze a content of one or more of the plurality of hydrocarbon feedstocks; and
(ii) predict, based at least in part on the content of one or more of the plurality of hydrocarbon feedstocks, one or more of:
(a) volume percent yield of fractionated rundown streams of the refining process; or
(b) yields of finished products of the refining process.

26. The distillation unit control assembly of claim 22, wherein:
(a) the one or more first processing units further comprise a vacuum distillation unit including a vacuum column positioned to receive a residue stream from the atmospheric column;
(b) the process controller is configured to control one or more of:
(i) separating, via the vacuum column, the residue stream into one or more intermediate process feedstocks;
(ii) separating one or more intermediate process feedstock samples from the one or more intermediate process feedstocks;
(iii) analyzing, via one or more of the first spectroscopic analyzer, the second spectroscopic analyzer, or the one or more additional spectroscopic analyzers, the one or more intermediate process feedstock samples; and
(iv) predicting, based at least in part on the analyzing of the one or more intermediate process feedstock samples, one or more intermediate process feedstock sample properties associated with the one or more intermediate process feedstock samples; and
(c) the prescriptively controlling is based at least in part on the one or more intermediate process feedstock sample properties.

27. A distillation unit control assembly to enhance control of a refining process associated with a petroleum refining operation, the distillation unit control assembly being in communication with one or more spectroscopic analyzers and one or more first processing units including one or more of fractionation units, the distillation unit control assembly being configured to:
predict one or more hydrocarbon feedstock sample properties associated with a hydrocarbon feedstock sample based at least in part on hydrocarbon feedstock sample spectra generated by the one or more spectroscopic analyzers;
predict one or more unit material sample properties associated with a unit material sample based at least in part on unit material sample spectra generated by the one or more spectroscopic analyzers, the one or more unit material sample properties being associated with one or more unit materials produced by the one or more first processing units and the one or more unit materials including one or more of intermediate materials or unit product materials; and
prescriptively control, during the refining process, based at least in part on the one or more hydrocarbon feedstock sample properties, and the one or more unit material sample properties, one or more of:
(a) the one or more hydrocarbon feedstock properties associated with hydrocarbon feedstock supplied to the one or more first processing units;
(b) one or more intermediate properties associated with the intermediate materials produced by one or more of the first processing units;
(c) operation of the one or more first processing units;
(d) one or more unit materials properties associated with the one or more unit materials produced by the one or more first processing units; or
(e) operation of one or more second processing units positioned downstream relative to the one or more first processing units,
so that the prescriptively controlling causes the refining process to produce one or more of:
(i) one or more intermediate materials each having one or more properties within a selected range of one or more target properties of the one or more intermediate materials;
(ii) one or more unit materials each having one or more properties within a selected range of one or more target properties of the one or more unit materials; or
(iii) one or more downstream materials each having one or more properties within a selected range of one or more target properties of the one or more downstream materials,
thereby to cause the refining process to achieve material outputs that more accurately and responsively converge on one or more of the target properties.

28. The distillation unit control assembly of claim 27, wherein the distillation unit control assembly is configured to prescriptively control, during the refining process, one or more operating parameters of the one or more first processing units against operating constraints associated with the one or more first processing units.

29. The distillation unit control assembly of claim 27, wherein the prescriptively controlling comprises controlling one or more process parameters, the one or more process parameters comprising one or more of:
(i) one or more hydrocarbon feedstock parameters;
(ii) a rate of supply of the hydrocarbon feedstock to the one or more first processing units;
(iii) a pressure of the hydrocarbon feedstock supplied to the one or more first processing units;
(iv) a preheating temperature of the hydrocarbon feedstock supplied to the one or more first processing units;
(v) one or more unit materials parameters;
(vi) a rate of supply of the one or more unit materials to the one or more second processing units;
(vii) a pressure of the one or more unit materials supplied to the one or more second processing units;
(viii) a preheating temperature of the one or more unit materials supplied to the one or more second processing units;
(ix) a temperature in the one or more first processing units;
(x) a pressure in the one or more first processing units;
(xi) a temperature in the one or more second processing units; or
(xii) a pressure in the one or more second processing units.
